(19) United States
(12) Patent Application Publication
(10) Pub. No.: US 2024/0299348 A1
(43) Pub. Date: Sep. 12, 2024

(54) MODULATORS OF TRPML, THEIR COMPOSITIONS AND METHODS OF USE

(71) Applicant: Caraway Therapeutics, Inc., Cambridge, MA (US)

(72) Inventors: Magdalene M. Moran, Watertown, MA (US); John Joseph Graziotto, Somerville, MA (US); Darby R. Schmidt, Arlington, MA (US); Rajesh R. Iyengar, West Newton, MA (US)

(21) Appl. No.: 18/260,670

(22) PCT Filed: Jan. 6, 2022
(86) PCT No.: PCT/US2022/011418

§ 371 Date: Jul. 7, 2023

Related U.S. Application Data
(60) Provisional application No. 63/135,236, filed on Jan. 8, 2021.

(51) Int. Cl. A61K 31/402; A61K 31/407; A61K 31/4418; A61K 31/4535; A61K 31/4709; A61K 31/495; A61K 45/06

(52) U.S. Cl. CPC A61K 31/402; A61K 31/407; A61K 31/4418; A61K 31/4535; A61K 31/4709; A61K 31/495; A61K 45/06

(57) ABSTRACT

The present disclosure relates to methods of treating a ciliopathy, the method comprising administering to a patient in need thereof a therapeutically effective amount of a compound or pharmaceutical composition thereof, wherein the compound modulates a TRPML ion channel. The disclosure also relates to pharmaceutical compounds of Formula (III) and subformulas thereof, or a pharmaceutically acceptable salt or composition thereof, and methods of their use for modulating TRPML ion channels, and for treating disorders.

### CROSS REFERENCE TO RELATED APPLICATIONS

This Application is a U.S. National Stage Application of International Patent Application No. PCT/US2022/011418, filed on Jan. 6, 2022, which claims the benefit of and priority to U.S. Provisional Patent Application No. 63/135,236, filed on Jan. 8, 2021, the contents of each of which are incorporated by reference herein in their entirety.

### TECHNICAL FIELD

The present disclosure relates to compounds and compositions which are modulators of TRPML-1 and are useful for treatment of polycystic kidney disease and related disorders.

### BACKGROUND

The lysosome, is a key organelle that serves as the cell's recycling center. In a highly regulated manner, it breaks down various biomaterials (proteins, lipids, and membranes) into smaller molecules or chemical building blocks, that the cell then employs for energy or as starting materials for new proteins or membranes [see e.g. de Duve, C, The lysosome turns fifty. Nat Cell Biol, 2005. 7(9): p. 847-9. Parkinson-Lawrence, E. J., et al., Lysosomal storage disease: revealing lysosomal function and physiology. Physiology (Bethesda), 2010. 25(2): p. 102-151. Lysosomal dysfunction due to mutations in the hydrolytic enzyme of lysosomal transport occur in the more than 50 genetically defined Lysosomal Storage Diseases. Interestingly, defects in lysosomal processing can have substantial effects on the function of the organelle beyond the actual enzyme that is mutated—in effect, the system can be gummed up—altering lysosomal degradation and membrane transport/trafficking, creating a positive feedback. Because lysosome storage is also seen in common neurodegenerative diseases such as Alzheimer's and Parkinson's, understanding the mechanisms underlying the positive feedback loop may provide therapeutic approaches not only for LSDs, but also for common sporadic neurodegenerative diseases. A lysosome-localized cation channel, TRPML1, has been recently identified as a key regulator of lysosomal function and membrane trafficking processes in the lysosome. Human mutations of TRPML1 cause an inherited lysosomal storage disease, Mucolipidosis IV. This disease is typified by neurodegenerative effects likely driven by the accumulation of lipids and other biomaterials in the cell.

Many reports suggest that TRPML1 is involved in multiple, key lysosomal functions. It can drive the translocation of the Transcription factor (TF)EB to the nucleus. TFEB regulates autophagy and lysosome biogenesis. Overexpression of TFEB has been reportedly induce cellular clearance in a number of lysosome storage diseases, including Pompe Disease, Cystinosis, multiple sulfatase deficiency, as well as common neurodegenerative diseases, including Parkinson's disease and Huntington's disease (Settembre, C, et al., Signals from the lysosome: a control centre for cellular clearance and energy metabolism. Nat Rev Mol Cell Biol, 2013. 14(5): p. 283-96). Therefore, activation of TRPML1 by TRPML1 agonists may also lead to cellular clearance in all the aforementioned diseases, providing therapeutic targets for these devastating diseases.

Recently, a potent synthetic agonist for TRPML1 has been reported [Shen, D., et al., Lipid storage disorders block lysosomal trafficking by inhibiting a TRP channel and lysosomal calcium release. Nat Commun, 2012. 3: p. 731]. This SF-51-related compound (Mucolipin Synthetic Agonist 1 or ML-SA1) that could induce significant [Ca2+] increases in HEK293 cells stably or transiently expressing TRPML1 protein that has been forced to the plasma membrane via deletion of its lysosomal targeting sequence. High concentrations of ML-SA1 (˜10 μM) are needed to effectively activate TRPMLs. Since that concentration is usually difficult to achieve in vivo, ML-SA1 cannot be used to treat the above TRPML related diseases. Liang et al. recently reported a new class of compounds as more potent TRPML activators [WO 2018/005713A1]. These compounds were thought to be useful in treating disorders related to TRPML activities such as lysosome storage diseases, muscular dystrophy, age-related common neurodegenerative diseases, ROS or oxidative stress related diseases, and aging. TRPML activators may also be useful in other disorders.

### SUMMARY

In an aspect, provided herein is a method of treating a ciliopathy, the method comprising administering to a patient in need thereof a therapeutically effective amount of a pharmaceutical composition or compound of the disclosure, or a pharmaceutically acceptable salt, tautomer, or stereoisomer thereof.

In an aspect, provided herein is a method of treating a ciliopathy, the method comprising administering to a patient in need thereof a therapeutically effective amount of a compound or pharmaceutical composition thereof, wherein the compound modulates a TRPML ion channel.

In an aspect, the present disclosure provides for a compound of Formula (III) and pharmaceutically acceptable salts, tautomers, and stereoisomers thereof:

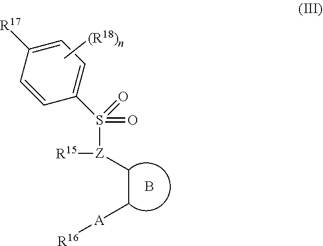

wherein
A is a divalent group with a nitrogen at one or both bonding sites, wherein the divalent group is selected from a mono or bicyclic heterocyclic ring containing at least one nitrogen atom, and a monocyclic heterocyclic ring containing at least one nitrogen with at least one optionally substituted amine bound to the heterocyclic ring;
B is an optionally substituted benzene ring or an optionally substituted C₃-C₇ cycloalkyl;
Z is N or CR¹⁵;
R¹⁵ is independently at each occurrence H or C₁-C₆ alkyl;
R¹⁶ is selected from a benzene ring, a C₃-C₆ cycloalkyl, or —C(O)R^{a}, wherein the benzene ring and cycloalkyl are optionally substituted;
R^{a} is selected from C₁-C₆ alkyl, C₁-C₈ cycloalkyl, C₁-C₈ cycloalkoxy, C₁-C₆ alkoxy, phenyl, or phenoxy;
R¹⁷ is selected from the group consisting of H, halo, hydroxy, cyano, C₁-C₆ alkyl, C₁-C₆ alkoxy, C₁-C₆ haloalkyl, —SO₂—C₁-C₆ alkyl, —SO₂—NR^{b}R^{c}, and C(O)NR^{b}R^{c};
R¹⁸ is independently selected at each occurrence from the group consisting of H, halo, hydroxy, cyano, C₁-C₆ alkyl, C₁-C₆ alkoxy, and C₁-C₆haloalkyl;
R^{b} and R^{c} are each independently selected at each occurrence from H and C₁-C₆ alkyl; and
n is 0 to 4;
provided that when B is a benzene ring, A is not piperazine, or R¹⁶ is not an optionally substituted benzene ring, or Z is CH.

The present disclosure further provides compositions comprising a compound of Formula (IIH) and a pharmaceutically acceptable excipient, diluent or carrier.

The present disclosure further provides methods of use of compounds or compositions comprising a compound of Formula (III).

### BRIEF DESCRIPTION OF THE FIGURES

FIG. 1 is a bar graph which shows total hexosylCeramides for HeLa cells with NPC1 gene knockout were reduced upon treatment with compound 101, when compared with DMSO vehicle.

FIG. 2 is a bar graph which shows total hexosylCeramides for HeLa cells with NPC2 gene knockout were reduced upon treatment with compound 101, when compared with DMSO vehicle.

FIG. 3 is a bar graph which shows TRPML1 activators from multiple chemical classes reduced cyst swelling in a murine in vitro model of Polycystic Kidney Disease.

### DETAILED DESCRIPTION

Accordingly, the present application provides compounds useful for treating ciliopathies and related diseases

In an aspect, the present disclosure provides for a compound of Formula (III) and pharmaceutically acceptable salts, tautomers, and stereoisomers thereof:

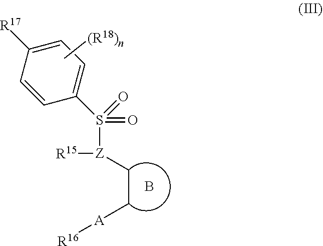

wherein
A is a divalent group with a nitrogen at one or both bonding sites, wherein the divalent group is selected from a mono or bicyclic heterocyclic ring containing at least one nitrogen atom, and a monocyclic heterocyclic ring containing at least one nitrogen with at least one optionally substituted amine bound to the heterocyclic ring;
B is an optionally substituted benzene ring or an optionally substituted C₃-C₇ cycloalkyl;
Z is N or CR¹⁵;
R¹⁵ is independently at each occurrence H or C₁-C₆ alkyl;
R¹⁶ is selected from a benzene ring, a C₃-C₆ cycloalkyl, or —C(O)R^{a}, wherein the benzene ring and cycloalkyl are optionally substituted;
R^{a} is selected from C₁-C₆ alkyl, C₁-C₈ cycloalkyl, C₁-C₈ cycloalkoxy, C₁-C₆ alkoxy, phenyl, or phenoxy;
R¹⁷ is selected from the group consisting of H, halo, hydroxy, cyano, C₁-C₆ alkyl, C₁-C₆ alkoxy, C₁-C₆ haloalkyl, —SO₂—C₁-C₆ alkyl, —SO₂—NR^{b}R^{c}, and C(O)NR^{b}R^{c};
R¹⁸ is independently selected at each occurrence from the group consisting of H, halo, hydroxy, cyano, C₁-C₆ alkyl, C₁-C₆ alkoxy, and C₁-C₆haloalkyl;
R^{b} and R^{c} are each independently selected at each occurrence from H and C₁-C₆ alkyl; and
n is 0 to 4;
provided that when B is a benzene ring, A is not piperazine, or R¹⁶ is not an optionally substituted benzene ring, or Z is CH.

In some embodiments, the compound is of formula (III)

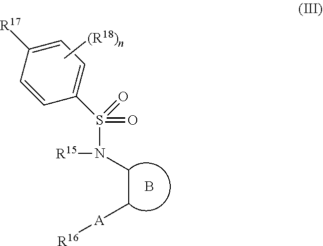

wherein
A is a divalent group with a nitrogen at one or both bonding sites, wherein the divalent group is selected from a mono or bicyclic heterocyclic ring containing at least two nitrogen atoms, and a monocyclic heterocyclic ring containing at least one nitrogen with at least one optionally substituted amine bound to the heterocyclic ring;
B is an optionally substituted C3-C7 cycloalkyl;
R¹⁵ is H or C₁-C₆ alkyl;
R¹⁶ is selected from a benzene ring, a C₃-C₆ cycloalkyl, or —C(O)R^{a}, wherein the benzene ring and cycloalkyl are optionally substituted;
R^{a} is selected from C₁-C₆ alkyl, C₁-C₈ cycloalkyl, C₁-C₈ cycloalkoxy, C₁-C₆ alkoxy, phenyl, and phenoxy;
R¹⁷ is selected from the group consisting of H, halo, hydroxy, cyano, C₁-C₆ alkyl, C₁-C₆ alkoxy, C₁-C₆ haloalkyl, —SO₂—C₁-C₆ alkyl, —SO₂—NRbRc, and C(O)NR^{b}R^{c};
R¹⁸ is independently selected at each occurrence from the group consisting of H, halo, hydroxy, cyano, C₁-C₆ alkyl, C₁-C₆ alkoxy, and C₁-C₆ haloalkyl;
R^{b} and R^{c} are each independently selected at each occurrence from H and C₁-C₆ alkyl; and n is 0 to 4.

In some embodiments, the compound is of formula (IIIa)

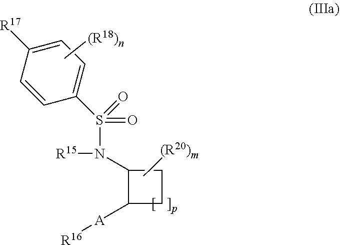

wherein
A is a divalent group with a nitrogen at one or both bonding sites, wherein the divalent group is selected from a mono or bicyclic heterocyclic ring containing at least two nitrogen atoms, and a monocyclic heterocyclic ring containing at least one nitrogen with at least one optionally substituted amine bound to the heterocyclic ring;
R¹⁵ is H or C₁-C₆ alkyl;
R¹⁶ is selected from a benzene ring, a C₃-C₆ cycloalkyl, or —C(O)R^{a}, wherein the benzene ring and cycloalkyl are optionally substituted;
R^{a} is selected from C₁-C₆ alkyl, C₁-C₈ cycloalkyl, C₁-C₈ cycloalkoxy, C₁-C₆ alkoxy, phenyl, and phenoxy;
R¹⁷ is selected from the group consisting of H, halo, hydroxy, cyano, C₁-C₆ alkyl, C₁-C₆ alkoxy, C₁-C₆ haloalkyl, —SO₂—C₁-C₆ alkyl, —SO₂—NR^{b}R^{c}, and C(O)NR^{b}R^{c};
R¹⁸ is independently selected at each occurrence from the group consisting of H, halo, hydroxy, cyano, C₁-C₆ alkyl, C₁-C₆ alkoxy, and C₁-C₆ haloalkyl;
R²⁰ is independently selected at each occurrence from the group consisting of H, halo, hydroxy, cyano, C₁-C₆ alkyl, C₁-C₆ alkoxy, and C₁-C₆ haloalkyl, NR^{b}R^{c}, C(O)—O—R^{b}, N(R^{b})—C(O)—O—R^{b}, N(R^{b})—C(O)—R^{b}, and C(O)NR^{b}R^{c};
R^{b} and R^{c} are each independently selected at each occurrence from H and C₁-C₆ alkyl;
n is 0 to 4;
m is 0 to 6; and
p is 1 to 4.

In some embodiments, the compound is of formula (IIIb)

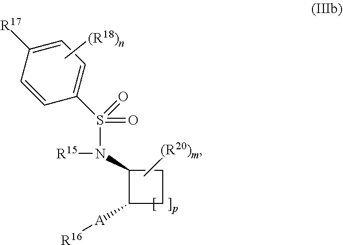

wherein the variable definitions are as provided in the disclosure.

In some embodiments, the compound is of formula (IIIc)

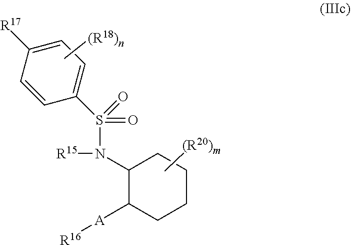

wherein
A is a divalent group with a nitrogen at one or both bonding sites, wherein the divalent group is selected from a mono or bicyclic heterocyclic ring containing at least two nitrogen atoms, and a monocyclic heterocyclic ring containing at least one nitrogen with at least one optionally substituted amine bound to the heterocyclic ring;
R¹⁵ is H or C₁-C₆ alkyl;
R¹⁶ is selected from a benzene ring, a C₃-C₆ cycloalkyl, or —C(O)R^{a}, wherein the benzene ring and cycloalkyl are optionally substituted;
R^{a} is selected from C₁-C₆ alkyl, C₁-C₈ cycloalkyl, C₁-C₈ cycloalkoxy, C₁-C₆ alkoxy, phenyl, and phenoxy;
R¹⁷ is selected from the group consisting of H, halo, hydroxy, cyano, C₁-C₆ alkyl, C₁-C₆ alkoxy, C₁-C₆ haloalkyl, —SO₂—C₁-C₆ alkyl, —SO₂—NR^{b}R^{c}, and C(O)NR^{b}R^{c};
R¹⁸ is independently selected at each occurrence from the group consisting of H, halo, hydroxy, cyano, C₁-C₆ alkyl, C₁-C₆ alkoxy, and C₁-C₆ haloalkyl;
R²⁰ is independently selected at each occurrence from the group consisting of H, halo, hydroxy, cyano, C₁-C₆ alkyl, C₁-C₆ alkoxy, and C₁-C₆ haloalkyl, NR^{b}R^{c}, C(O)—O—R^{b}, N(R^{b})—C(O)—O—R^{b}, N(R^{b})—C(O)—R^{b}, and C(O)NR^{b}R^{c};
R^{b} and R^{c} are each independently selected at each occurrence from H and C₁-C₆ alkyl;
n is 0 to 4; and
m is 0 to 6.

In some embodiments, the compound is of formula (IIId)

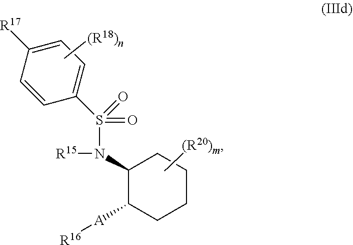

wherein the variable definitions are as provided in the disclosure.

In some embodiments, the compound is of formula (IIIe)

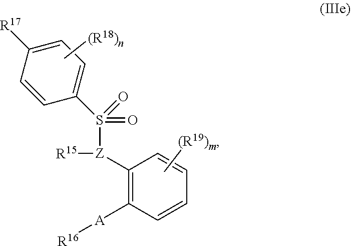

wherein
A is a divalent group with a nitrogen at one or both bonding sites, wherein the divalent group is selected from a mono or bicyclic heterocyclic ring containing at least two nitrogen atoms, and a monocyclic heterocyclic ring containing at least one nitrogen with at least one optionally substituted amine bound to the heterocyclic ring;
Z is N or CR¹⁵;
R¹⁵ is independently at each occurrence H or C₁-C₆ alkyl;
R¹⁶ is selected from a benzene ring, a C₃-C₆ cycloalkyl, or —C(O)R^{a}, wherein the benzene ring and cycloalkyl are optionally substituted;
R^{a} is selected from C₁-C₆ alkyl, C₁-C₈ cycloalkyl, C₁-C₈ cycloalkoxy, C₁-C₆ alkoxy, phenyl, and phenoxy;
R¹⁷ is selected from the group consisting of H, halo, hydroxy, cyano, C₁-C₆ alkyl, C₁-C₆ alkoxy, C₁-C₆ haloalkyl, —SO₂—C₁-C₆ alkyl, —SO₂—NR^{b}R^{c}, and C(O)NR^{b}R^{c};
R¹⁸ is independently selected at each occurrence from the group consisting of H, halo, hydroxy, cyano, C₁-C₆ alkyl, C₁-C₆ alkoxy, and C₁-C₆ haloalkyl;
R¹⁹ is independently selected at each occurrence from the group consisting of H, halo, hydroxy, cyano, C₁-C₆ alkyl, C₁-C₆ alkoxy, and C₁-C₆ haloalkyl, NR^{b}R^{c}, C(O)—O—Rb, N(R^{b})—C(O)—O—R^{b}, N(R^{b})—C(O)—R^{b}, and C(O)NR^{b}R^{c};
R^{b} and R^{c} are each independently selected at each occurrence from H and C₁-C₆ alkyl;
n is 0 to 4;
m is 0 to 6; and
provided that A is not piperazine, or R¹⁶ is not an optionally substituted benzene ring, or Z is CH.

In some embodiments, the compound is of formula (IIIf)

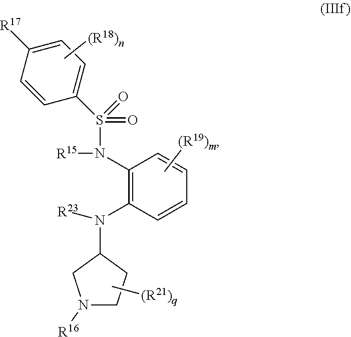

wherein
R¹⁵ is H or C₁-C₆ alkyl;
R¹⁶ is selected from a benzene ring, a C₃-C₆ cycloalkyl, or —C(O)R^{a}, wherein the benzene ring and cycloalkyl are optionally substituted;
R^{a} is selected from C₁-C₆ alkyl, C₁-C₈ cycloalkyl, C₁-C₈ cycloalkoxy, C₁-C₆ alkoxy, phenyl, and phenoxy;
R¹⁷ is selected from the group consisting of H, halo, hydroxy, cyano, C₁-C₆ alkyl, C₁-C₆ alkoxy, C₁-C₆ haloalkyl, —SO₂—C₁-C₆ alkyl, —SO₂—NR^{b}R^{c}, and C(O)NR^{b}R^{c};
R¹⁸ is independently selected at each occurrence from the group consisting of H, halo, hydroxy, cyano, C₁-C₆ alkyl, C₁-C₆ alkoxy, and C₁-C₆ haloalkyl;
R¹⁹ is independently selected at each occurrence from the group consisting of H, halo, hydroxy, cyano, C₁-C₆ alkyl, C₁-C₆ alkoxy, and C₁-C₆ haloalkyl, NR^{b}R^{c}, C(O)—O—R^{b}, N(R^{b})—C(O)—O—R^{b}, N(R^{b})—C(O)—R^{b}, and C(O)NR^{b}R^{c};
R²¹ is independently selected at each occurrence from the group consisting of H, halo, hydroxy, cyano, C₁-C₆ alkyl, C₁-C₆ alkoxy, and C₁-C₆ haloalkyl, NRbRc, C(O)—O—Rb, N(R^{b})—C(O)—O—R^{b}, N(R^{b})—C(O)—R^{b}, and C(O)NR^{b}R^{c};
R²³ is H or C₁-C₆ alkyl;
R^{b} and R^{c} are each independently selected at each occurrence from H and C₁-C₆ alkyl;
n is 0 to 4;
m is 0 to 6; and
q is 0 to 4.

In some embodiments, the compound is of formula (IIIg)

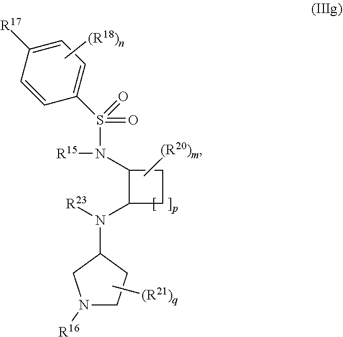

wherein
R¹⁵ is H or C₁-C₆ alkyl;
R¹⁶ is selected from a benzene ring, a C₃-C₆ cycloalkyl, or —C(O)R^{a}, wherein the benzene ring and cycloalkyl are optionally substituted;
R^{a} is selected from C₁-C₆ alkyl, C₁-C₈ cycloalkyl, C₁-C₈ cycloalkoxy, C₁-C₆ alkoxy, phenyl, and phenoxy;
R¹⁷ is selected from the group consisting of H, halo, hydroxy, cyano, C₁-C₆ alkyl, C₁-C₆ alkoxy, C₁-C₆ haloalkyl, —SO₂—C₁-C₆ alkyl, —SO₂—NR^{b}R^{c}, and C(O)NR^{b}R^{c};
R¹⁸ is independently selected at each occurrence from the group consisting of H, halo, hydroxy, cyano, C₁-C₆ alkyl, C₁-C₆ alkoxy, and C₁-C₆ haloalkyl;
R²⁰ is independently selected at each occurrence from the group consisting of H, halo, hydroxy, cyano, C₁-C₆ alkyl, C₁-C₆ alkoxy, and C₁-C₆ haloalkyl, NR^{b}R^{c}, C(O)—O—R^{b}, N(R^{b})—C(O)—O—R^{b}, N(R^{b})—C(O)—R^{b}, and C(O)NR^{b}R^{c};
R^{b} and R^{c} are each independently selected at each occurrence from H and C₁-C₆ alkyl;
n is 0 to 4;
m is 0 to 6; and
p is 1 to 4.

In some embodiments, A is a 6-7 membered monocyclic heterocycle with two nitrogen ring atoms, a 7-12-membered bicyclic heterocycle with two nitrogen ring atoms, or an amino pyrrolidine. In some embodiments, A is a piperazine, a 1,4-diazacycloheptane, a 3-aminopyrrolidine, a 2,7-diazaspiro[4.4]nonane, a, 2,6-diazaspiro[3.4]octane, or a 2,5-diazabicyclo[2.2.1]heptane. In some embodiments, A is a 7-12-membered bicyclic heterocycle with two nitrogen ring atoms, or an amino pyrrolidine. In some embodiments, A is a 3-aminopyrrolidine, a 2,7-diazaspiro[4.4]nonane, a, 2,6-diazaspiro[3.4]octane, or a 2,5-diazabicyclo[2.2.1]heptane. In some embodiments, A is substituted with 0-4 R²¹ groups. In some embodiments, A is unsubstituted.

In some embodiments, B is a benzene ring. In some embodiments, B is a C₃-C₇ cycloalkyl. In some embodiments B is a trans cyclohexane or trans cycloheptane. In some embodiments, B is a trans cyclohexane. In some embodiments, B is substituted with 0-6 R²⁰ groups. In some embodiments, B is unsubstituted.

In some embodiments, R¹⁶ is a benzene ring substituted with 0-4 R¹⁸ groups. In some embodiments, R¹⁶ is unsubstituted. In some embodiments R¹⁶ is a C₃-C₆ cycloalkyl, or —C(O)R^{a}, wherein the cycloalkyl is optionally substituted. In some embodiments the cycloalkyl is unsubstituted.

In some embodiments, R¹⁶ is selected from the group consisting of

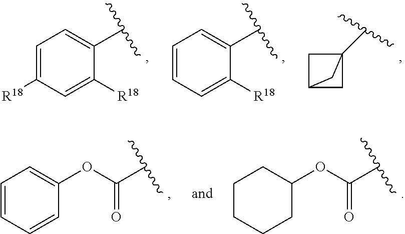

In some embodiments, R^{a} is selected from C₁-C₆ alkyl, C₁-C₈ cycloalkyl, C₁-C₈ cycloalkoxy, C1-C6 alkoxy, phenyl, and phenoxy. In some embodiments, R^{a} is selected from C1-C8 cycloalkoxy, C1-C6 alkoxy, and phenoxy.

In some embodiments, R¹⁷ is selected from the group consisting of C₁-C₆ alkyl, —SO₂-C₁-C₆ alkyl, and —SO₂—NR^{b}R^{c}.

In some embodiments, R¹⁸ is independently selected at each occurrence from the group consisting of halo and C₁-C₆ alkyl. In some embodiments, R¹⁸ is independently selected at each occurrence from the group consisting of fluoro and chloro.

In some embodiments, R¹⁹ is independently selected at each occurrence from the group consisting of halo and C₁-C₆ alkyl.

In some embodiments, R²⁰ is independently selected at each occurrence from the group consisting of halo and C₁-C₆ alkyl.

In some embodiments, R²¹ is independently selected at each occurrence from the group consisting of halo and C₁-C₆ alkyl.

In some embodiments, R¹⁵ is C₁-C₆ alkyl. In some embodiments, R¹⁵ is methyl.

The present application further provides pharmaceutical compositions comprising a compound provided herein, or a pharmaceutically acceptable salt thereof, and at least one pharmaceutically acceptable carrier. The present application further provides methods of modulating a TRPML channel in a subject, the method comprising administering to the subject a therapeutically effective amount of a compound provided herein, or a pharmaceutically acceptable salt thereof.

In an aspect, provided herein is a method of treating a ciliopathy, the method comprising administering to a patient in need thereof a therapeutically effective amount of a compound or pharmaceutical composition thereof, wherein the compound modulates a TRPML ion channel.

In some embodiments, the compound activates the TRPML ion channel. In some embodiments the TRPML ion channel is TRPML1.

In some embodiments, the compound achieves a maximal current obtained with 30 μM ML-SA1 in a patch clamp assay for TRPML1 which is at least 10 fold the maximal current achieved for any other TRPML channel.

In some embodiments, the ciliopathy is selected from the group consisting of polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy, Ellis van Creveld syndrome, Sensenbrenner syndrome, and primary ciliary dyskinesia.

In some embodiments, the ciliopathy is polycystic kidney disease or pancreatic cysts associated with autosomal dominant polycystic kidney disease. In some embodiments, the ciliopathy is autosomal dominant polycystic kidney disease, autosomal recessive polycystic kidney disease, or pancreatic cysts associated with autosomal dominant polycystic kidney disease. In some embodiments, the ciliopathy is autosomal dominant polycystic kidney disease.

In some embodiments, the method further comprising use of a second therapeutic agent.

In some embodiments, the second therapeutic agent is selected from the group consisting of an mTOR inhibitor, V2 receptor antagonist, tyrosine kinase inhibitor, somatostatin analog, glucosylceramide synthase inhibitor, microRNA-17 inhibitor, siRNA against p53, KEAP1-Nrf2 activator, xanthine oxidase inhibitor, PPARγ agonist, metformin, and beta hydroxybutyrate.

In some embodiments, the second therapeutic agent is selected from the group consisting of tolvaptan, lixivaptan, mozavaptan, satavaptan, sirolimus, tacrolimus, everolimus, bosutinib, tesavatinib, imatinib, gefitinib, erlotinib, dasatinib, octreotide, pasireotide, venglustat, eliglustat, miglustat, microRNA-17 inhibitor, bardoxolone methyl, allopurinol, oxypurinol, pioglitazone, rosiglitazone, lobeglitazone, metformin, and beta hydroxybutyrate.

In some embodiments, the second therapeutic agent is selected from the group consisting of an immunomodulator, a calcineurin inhibitor, a renin angiotensin aldosterone system inhibitor, an antiproliferative agent, an alkylating agent, a corticosteroid, an angiotensin converting enzyme inhibitor, an adrenocorticotropic hormone stimulant, an angiotensin receptor blocker, a sodium-glucose transport protein 2 inhibitor, a dual sodium-glucose transport protein 1/2 inhibitor, a nuclear Factor-1 (erythroid-derived 2)-like 2 agonist, a chemokine receptor 2 inhibitor, a chemokine receptor 5 inhibitor, an endothelin 1 receptor antagonist, a beta blocker, a mineralocorticoid receptor antagonist, a loop or thiazide diuretic, a calcium channel blocker, a statin, a short-intermediate or long-acting insulin, a dipeptidyl peptidase 4 inhibitor, a glucagon-like peptide 1 receptor agonist, a sulfonylurea, an apoptosis signal-regulating kinase-1, a chymase inhibitor, a selective gly cation inhibitor, a renin inhibitor, an interleukin-33 inhibitor, a farnesoid X receptor agonist, a soluble guanylate cyclase stimulator, a thromboxane receptor antagonist, a xanthine oxidase inhibitor, an erythropoietin receptor agonist, a cannabinoid receptor type 1 inverse agonist, a NADPH oxidase inhibitor, an anti-vascular endothelial growth factor B, an anti-fibrotic agent, a neprilysin inhibitor, a dual CD80/CD86 inhibitor, a CD40 antagonist, a cellular cholesterol and lipid blocker, a PDGFR antagonist, a Slit guidance ligand 2, an APOL1 inhibitor, an Nrl2 activator/NF-kB inhibitor, a somatostatin receptor agonist, a PPAR gamma agonist, a AMP activated protein kinase stimulator, a tyrosine kinase inhibitor, a glucosylceramide synthase inhibitor, an arginine vasopressin receptor 2 antagonist, a xanthine oxidase inhibitor, a vasopressin receptor 2 antagonist, anti-amyloid beta antibodies, anti-Tau antibodies, anti-synuclein antibodies, dopamine precursors (e.g. L-DOPA), dopamine agonists (e.g. bromocriptine, cabergoline, pergolide, pramipexole and apomorphine), MAO-B inhibitors (e.g. rasagiline and selegiline), anticholinergics (e.g. orphenadrine, procyclidine and trihexyphenidyl), enhancers of b-glucocerebrosidase activity (e.g. ambroxol and afegostat), amantadine, and agents capable of treating Alzheimer's (e.g., acetylcholinesterase inhibitors such as tacrine, rivastigmine, galantamine, donepezil, and memantine).

In some embodiments, the second therapeutic agent is selected from the group consisting of COX inhibitors including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam; treatments for pulmonary hypertension including prostanoids (epoprostenol, iloprost, and treprostinil), endothelin receptor antagonists (bosentan, ambrisentan, and macitentan), phosphodiesterase-5 inhibitors (sildenafil and tadalafil), and sGC stimulators (riociguat); rho-kinase inhibitors, such as Y-27632, fasudil, and H-1152P; epoprostenol derivatives, such as prostacyclin, treprostinil, beraprost, and iloprost; serotonin blockers, such as sarpogrelate; endothelin receptor antagonists, such as besentan, sitaxsentan, ambrisentan, and TBC3711; PDE inhibitors, such as sildenafil, tadalafil, udenafil, and vardenafil; calcium channel blockers, such as amlodipine, bepridil, clentiazem, diltiazem, fendiline, gallopamil, mibefradil, prenylamine, semotiadil, terodiline, verapamil, aranidipine, bamidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, and perhexiline; tyrosine kinase inhibitors, such as imatinib: inhaled nitric oxide and nitric oxide-donating agents, such as inhaled nitrite; IκB inhibitors, such as IMD 1041; prostacyclin receptor agonists, such as selexipag: stimulators of hematopoiesis, such as TXA 127 (angiotensin (1-7)), darbepoetin alfa, erythropoetin, and epoetin alfa; anticoagulants and platelet-inhibiting agents; and diuretics; dietary and nutritional supplements such as acetyl-L-carnitine, octacosanol, evening primrose oil, vitamin B6, tyrosine, phenylalanine, vitamin C, L-dopa; immunosuppressants (for transplants and autoimmune-related RKD); anti-hypertensive drugs (for high blood pressure-related RKD, e.g., angiotensin-converting enzyme inhibitors and angiotensin receptor blockers); insulin (for diabetic RKD); lipid/cholesterol-lowering agents (e.g., HMG-CoA reductase inhibitors such as atorvastatin or simvastatin); and treatments for hyperphosphatemia or hyperparathyroidism associated with CKD (e.g., sevelamer acetate, cinacalcet).

In some embodiments, the compound is a compound disclosed in the specification, including in a reference incorporated therein. In some embodiments, the compound is a compound of formula III, or a subformula thereof.

In some embodiments, the present application further provides a method of treating a disease or disorder in a subject, the method comprising:
(a) detecting a disease or disorder associated with modulating a TRPML ion channel; and
(b) administering to the subject a therapeutically effective amount of a compound provided herein, or a pharmaceutically acceptable salt thereof.

In certain embodiments, exemplary compounds of Formula (III) include the compounds described in Table 1 and in the Examples, as well as pharmaceutically acceptable salts, solvates, hydrates, tautomers, and stereoisomers thereof. In certain embodiments, compounds of the disclosure include compounds of tables 1 and 2, in addition to compounds disclosed in references incorporated herein, as well as other compounds which are effective modulators of TRPML ion channels.

This disclosure is not limited in its application to the details of the methods and compositions described herein. Also, the phraseology and terminology used herein is for the purpose of description and should not be regarded as limiting.

### Definitions

### Selected Chemical Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, propyl, butyl, and pentyl.

For compounds of the disclosure in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

In case a compound of the present disclosure is depicted in form of a chemical name and as a formula in case of any discrepancy the formula shall prevail.

An asterisk or wavy line may be used in sub-formulas to indicate the bond which is connected to the core molecule as defined.

The term “substituted,” as used herein, means that any one or more hydrogens on the designated atom, usually a carbon, oxygen, or nitrogen atom, is replaced with a selection from the indicated group, provided that the designated atom's normal valency is not exceeded, and that the substitution results in a stable compound. When a substituent is keto or oxo (i.e., =0), then 2 hydrogens on the atom are replaced. Ring double bonds, as used herein, are double bonds that are formed between two adjacent ring atoms (e.g., C═C, C═N, N═N, etc.).

As used herein, “alkyl” is intended to include both branched and straight-chain saturated aliphatic hydrocarbon groups having the specified number of carbon atoms. For example, C₁-C₄ alkyl is intended to include C₁, C₂, C₃, and C₄. C₁-C₆ alkyl is intended to include C₁, C₂, C₃, C₄, C₅, and C₆ alkyl groups and C₁-C₅ alkyl is intended to include C₁, C₂, C₃, C₄, C₅, C₆, C₇, and C₈ alkyl groups. Some examples of alkyl include, but are not limited to, methyl, ethyl, n-propyl, iso-propyl, n-butyl, s-butyl, t-butyl, n-pentyl, s-pentyl, n-hexyl, n-heptyl, and n-octyl.

As used herein, “alkenyl” is intended to include hydrocarbon chains of either straight or branched configuration and one or more unsaturated carbon-carbon bond that can occur in any stable point along the chain, such as ethenyl and propenyl. For example, C₂-C₆ alkenyl is intended to include C₂, C₃, C₄, C₅, and C₆ alkenyl groups and C₂-C₆ alkenyl is intended to include C₂, C₃, C₄, C₅, C₆, C₇, and C₈ alkenyl groups.

As used herein, “alkylene” is intended to include moieties which are diradicals, i.e., having two points of attachment. A non-limiting example of such an alkylene moiety that is a diradical is —CH₂CH₂—, i.e., a C₂ alkyl group that is covalently bonded via each terminal carbon atom to the remainder of the molecule. The alkylene diradicals are also known as “alkylenyl” radicals. Alkylene groups can be saturated or unsaturated (e.g., containing —CH═CH— or —C≡C— subunits), at one or several positions. In some embodiments, alkylene groups include 1 to 9 carbon atoms (for example, 1 to 6 carbon atoms, 1 to 4 carbon atoms, or 1 to 2 carbon atoms). Some examples of alkylene groups include, but are not limited to, methylene, ethylene, n-propylene, iso-propylene, n-butylene, iso-butylene, sec-butylene, tert-butylene, n-pentylene, iso-pentylene, sec-pentylene and neo-pentylene.

As used herein, “cycloalkyl” is intended to include saturated or unsaturated nonaromatic ring groups, such as cyclopropyl, cyclobutyl, cyclopentyl, or cyclohexyl. For example, the term “C3-C8 cycloalkyl” is intended to include C₃, C₄, C₅, C₆, C₇, and C₈ cycloalkyl groups. Cycloalkyls may include multiple spiro- or fused or bridged rings. For example, cycloalkyl can include, but is not limited to, spiro butyl, pentyl, hexyl, heptyl, octyl, nonyl, or decyl groups, bicyclo butyl, pentyl, hexyl, heptyl, octyl, nonyl, or decyl groups, adamantyl groups, and norbornyl groups.

As used herein, the term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, or Se), unless specified otherwise. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, tetrahyrofuranyl, oxiranyl, azetidinyl, oxetanyl, thietanyl, 1,2,3,6-tetrahydropyridinyl, tetrahydropyranyl, dihydropyranyl, pyranyl, morpholinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl and the like.

As used herein, “amine” or “amino” refers to unsubstituted —H2 unless otherwise specified. As used herein, “halo” or “halogen” refers to fluoro, chloro, bromo, and iodo substituents.

As used herein, “haloalkyl” is intended to include both branched and straight-chain saturated aliphatic hydrocarbon groups having the specified number of carbon atoms, substituted with one or more halogen (for example —CᵥF_{w}H_{2v-w+1} wherein v=1 to 3 and w=1 to (2v+1)). Examples of haloalkyl include, but are not limited to, trifluoromethyl, trichlorom ethyl, pentafluoroethyl, and pentachloroethyl.

The term “haloalkoxy” as used herein refers to an alkoxy group, as defined herein, which is substituted one or more halogen. Examples of haloalkoxy groups include, but are not limited to, trifluoromethoxy, difluoromethoxy, pentafluoroethoxy, trichloromethoxy, etc.

As used herein, “alkoxyl” or “alkoxy” refers to an alkyl group as defined above with the indicated number of carbon atoms attached through an oxygen bridge. C₁-C₆ alkoxy, is intended to include C₁, C₂, C₃, C₄, C₅, and C₆ alkoxy groups. C₁-C₈ alkoxy, is intended to include C₁, C₂, C₃, C₄, C₅, C₆, C₇, and C₈ alkoxy groups. Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy.

As used herein, “aryl” includes groups with aromaticity, including “conjugated,” or multicyclic systems with at least one aromatic ring and do not contain any heteroatom in the ring structure. Aryl may be monocyclic or polycyclic (e.g., having 2, 3 or 4 fused rings). The term “Cn-m aryl” refers to an aryl group having from n to m ring carbon atoms. In some embodiments, aryl groups have from 6 to 10 carbon atoms. In some embodiments, the aryl group is phenyl or naphthyl.

As used herein, the terms “aromatic heterocycle,” “aromatic heterocyclic” or “heteroaryl” ring are intended to mean a stable 5, 6, 7, 8, 9, 10, 11, or 12-membered monocyclic or bicyclic aromatic ring which consists of carbon atoms and one or more heteroatoms, e.g., 1 or 1-2 or 1-3 or 1-4 or 1-5 or 1-6 heteroatoms, independently selected from nitrogen, oxygen, and sulfur. In the case of bicyclic aromatic heterocyclic or heterocycle or heteroaryl rings, only one of the two rings needs to be aromatic (e.g., 2,3-dihydroindole), though both can be (e.g., quinoline). The second ring can also be fused or bridged as defined above for heterocycles. The nitrogen atom can be substituted or unsubstituted (i.e., N or R wherein R is H or another substituent, as defined). The nitrogen and sulfur heteroatoms can optionally be oxidized (i.e., N→0) and S(0)_{P}, wherein p=1 or 2). In certain compounds, the total number of S and O atoms in the aromatic heterocycle is not more than 1.

Examples of aromatic heterocycles, aromatic heterocyclics or heteroaryls include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, benzooxadiazoly, carbazolyl, 4aH-carbazolyl, carbolinyl, cinnolinyl, furazanyl, imidazolyl, imidazolonyl, 1H-indazolyl, indolizinyl, indolyl, 3H-indolyl, isobenzofuranyl, isochromanyl, isoindazolyl, isoindolyl, isoquinolinyl, isothiazolyl, isoxazolyl, methylbenztriazolyl, methylfuranyl, methylimidazolyl, methylthiazolyl, naphthyridinyl, oxadiazolyl, 1,2,3-oxadiazolyl, 1,2,4-oxadiazolyl, 1,2,5-oxadiazolyl, 1,3,4-oxadiazolyl, oxazolyl, phenanthridinyl, phenanthrolinyl, phenazinyl, phenothiazinyl, phenoxathinyl, phenoxazinyl, phthalazinyl, pteridinyl, purinyl, pyrazinyl, pyrazolyl, pyridazinyl, pyridooxazolyl, pyridoimidazolyl, pyridothiazolyl, pyridinyl, pyridinonyl, pyridyl, pyrimidinyl, 2H-pyrrolyl, pyrrolyl, quinazolinyl, quinolinyl, 4H-quinolizinyl, quinoxalinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, triazolopyrimidinyl, 1,2,3-triazolyl, 1,2,4-triazolyl, 1,2,5-triazolyl, and 1,3,4-triazolyl.

The term “hydroxyalkyl” means an alkyl group as defined above, where the alkyl group is substituted with one or more OH groups. Examples of hydroxyalkyl groups include HO—CH₂—, HO—CH₂—CH₂— and CH₃—CH(OH)—.

The term “cyano” as used herein means a substituent having a carbon atom joined to a nitrogen atom by a triple bond, i.e., C≡N.

As used herein, “oxo” is means a “═O” group.

As used herein, the phrase “pharmaceutically acceptable” refers to those compounds or tautomers thereof, or salts thereof, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein, “pharmaceutically acceptable salts” refer to derivatives of the disclosed compounds or tautomers thereof, wherein the parent compound or a tautomer thereof, is modified by making of the acid or base salts thereof of the parent compound or a tautomer thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of die parent compound, or a tautomer thereof, formed, for example, from non-toxic inorganic or organic acids. For example, such conventional non-toxic salts include, but are not limited to, those derived from inorganic and organic acids selected from 2-acetoxybenzoic, 2-hydroxy ethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycoloylarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, and toluene sulfonic.

The pharmaceutically acceptable salts of the present disclosure can be synthesized from the parent compound or a tautomer thereof that contains a basic or acidic moiety by conventional chemical methods. Generally, such pharmaceutically acceptable salts can be prepared by reacting the free acid or base forms of these compounds or tautomers thereof with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, PA. USA, p. 1445 (1990).

As used herein, “stable compound” and “stable structure” are meant to indicate a compound that is sufficiently robust to survive isolation to a useful degree of purity from a reaction mixture, and formulation into an efficacious therapeutic agent.

As used herein, the term “treating” refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing or preventing the further development of a disorder, and/or reducing the severity of symptoms that will or are expected to develop.

As used herein, “unsaturated” refers to compounds having at least one degree of unsaturation (e.g., at least one multiple bond) and includes partially and fully unsaturated compounds.

As used herein, the term “effective amount” refers to an amount of a compound or a pharmaceutically acceptable salt of the compound or tautomer (including combinations of compounds and/or tautomers thereof, and/or pharmaceutically acceptable salts of said compound or tautomer) of the present disclosure that is effective when administered alone or in combination as an antimicrobial agent. For example, an effective amount refers to an amount of the compound or tautomer thereof, or a pharmaceutically acceptable salt said compound or tautomer that is present in a composition, a formulation given to a recipient patient or subject sufficient to elicit biological activity.

In the specification, the singular forms also include the plural, unless the context clearly dictates otherwise. Unless defined otherwise, all technical and scientific terms used herein have the same meaning as commonly understood by one of ordinary skill in the art to which this disclosure belongs. In the case of conflict, the present specification will control. As used herein, “mammal” refers to human and non-human patients.

As used herein, the term “formulae of the disclosure” or “compound of formula (III)” includes compounds of formula (III) and compounds of all subformulas thereof.

As used herein, the term “compound of the disclosure” or “compound disclosed herein” includes one or more compounds of the formulae of the disclosure or a compound explicitly disclosed herein.

All percentages and ratios used herein, unless otherwise indicated, are by weight.

Throughout the description, where compositions are described as having, including, or comprising specific components, or where processes are described as having, including, or comprising specific process steps, it is contemplated that compositions of the present disclosure also consist essentially of, or consist of, the recited components, and that the processes of the present disclosure also consist essentially of, or consist of, the recited processing steps. Further, it should be understood that the order of steps or order for performing certain actions are immaterial so long as the disclosure remains operable. Moreover, two or more steps or actions can be conducted simultaneously.

Contemplated equivalents of the compounds described above include compounds which otherwise correspond thereto, and which have the same general properties thereof (e.g., the ability to modulate a TRPML channel), wherein one or more simple variations of substituents are made which do not adversely affect the efficacy of the compound. In general, the compounds of the present disclosure may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The term, “treat” or “treatment,” as used herein, refers to the application or administration of a compound, alone or in combination with, an additional agent to a subject, e.g., a subject who has a disorder (e.g., a disorder as described herein), a symptom of a disorder, or a predisposition toward a disorder, with the purpose to cure, heal, alleviate, relieve, alter, remedy, ameliorate, improve or affect the disorder.

As used herein, the term “subject” is intended to include human and non-human animals. Exemplary human subjects include a human subject having a disorder, e.g., a disorder described herein. The term “non-human animals” of the disclosure includes all vertebrates, e.g., non-mammals (such as chickens, amphibians, reptiles) and mammals, such as non-human primates, domesticated and/or agriculturally useful animals, e.g., sheep, dog, cat, cow, pig, etc.

The terms “antagonist” and “inhibitor” are used interchangeably to refer to an agent that decreases or suppresses a biological activity.

The term “hydrate” as used herein, refers to a compound formed by the union of water with the parent compound.

The term “preventing,” when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “solvate” as used herein, refers to a compound formed by solvation (e.g., a compound formed by the combination of solvent molecules with molecules or ions of the solute).

Another aspect of the disclosure features a pharmaceutical preparation suitable for use in a human patient, or for veterinary use, comprising an effective amount of a compound of the disclosure (or a salt thereof, or a solvate, hydrate, oxidative metabolite or prodrug of the compound or its salt), and one or more pharmaceutically acceptable excipients. The disclosure further contemplates the use of compounds of the disclosure in the manufacture of a medicament or pharmaceutical preparation to treat or reduce the symptoms of any of the diseases or conditions provided in the specification. The compounds of the disclosure for use in treating a particular disease or condition can be formulated for administration via a route appropriate for the particular disease or condition.

Compounds of the disclosure can be administered alone or in combination with another therapeutic agent. For instance, the compounds of the disclosure can be administered conjointly with one or more of an agent for treating polycystic kidney disease, etc.

Compounds of the disclosure can be administered topically, orally, transdermally, rectally, vaginally, parentally, intranasally, intrapulmonary, intraocularly, intravenously, intramuscularly, intraarterially, intrathecally, intracapsularly, intraorbitally, intracardiacly, intradermally, intraperitoneally, transtracheally, subcutaneously, subcuticularly, intraarticularly, subcapsularly, subarachnoidly, intraspinally, intrasternally, sublingually, or by inhalation.

In some embodiments, compounds of the disclosure can be administered topically.

In some embodiments, compounds of the disclosure can be administered orally.

In some embodiments, compounds of the disclosure can be administered parentally.

Compounds of the disclosure include molecules having an aqueous solubility suitable for oral or parenteral (e.g., intravenous) administration leading to or resulting in the treatment of a disorder described herein, for example the treatment of pain. In some embodiments, the compound is formulated into a composition suitable for oral administration.

In some embodiments, a compound of the disclosure can be administered as part of an oral or parenteral (e.g., intravenous) pharmaceutical composition to treat a disorder described herein in a therapeutically effective manner.

Certain compounds disclosed herein may exist in particular geometric or stereoisomeric forms. The present disclosure contemplates all such compounds, including cis- and trans-isomers, R- and S-enantiomers, diastereomers, (d)-isomers, (l)-isomers, the racemic mixtures thereof, and other mixtures thereof, as falling within the scope of the disclosure. For example, if one chiral center is present in a molecule, the disclosure includes racemic mixtures, enantiomerically enriched mixtures, and substantially enantiomerically or diastereomerically pure compounds. The composition can contain, e.g., more than 50%, more than 60%, more than 70%, more than 80%, more than 90%, more than 95%, or more than 99% of a single enantiomer or diastereomer. Additional asymmetric carbon atoms may be present in a substituent such as an alkyl group. All such isomers, as well as mixtures thereof, are intended to be included in this disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer.

$$ee = ( 90 - 10 ) / 100 = 80 \% .$$

Thus, a composition containing 90% of one enantiomer and 10% of the other enantiomer is said to have an enantiomeric excess of 80%.

The “diastereomeric excess” or “% diastereomeric excess” of a composition can be calculated using the equation shown below. In the example shown below a composition contains 90% of one diastereomer, and 10% of another enantiomer.

$$ee = ( 90 - 10 ) / 100 = 80 \% .$$

Thus, a composition containing 90% of one diastereomer and 10% of the other diastereomer is said to have an diastereomeric excess of 80%.

Certain compounds disclosed herein can exist in unsolvated forms as well as solvated forms, including hydrated forms. In general, the solvated forms are equivalent to unsolvated forms and are encompassed within the scope of the present disclosure. Certain compounds disclosed herein may exist in multiple crystalline or amorphous forms. In general, all physical forms are equivalent for the uses contemplated by the present disclosure and are intended to be within the scope of the present disclosure.

### TRPML Modulators and Disease

The present disclosure features compounds (e.g., compounds of Formula (III)) useful for the modulation of TRPML ion channels and pharmaceutically acceptable salts, solvates, hydrates, tautomers, stereoisomers, isotopically labeled derivatives thereof, as well as compositions thereof, for the treatment of a disease or disorder related to the TRPML channel.

In addition to compounds of formula (III), modulators of the TRPML channels have been reported in several publications, including WO2018005713 and WO2018208630, which are incorporated herein in their entirety.

The present application further provides pharmaceutical compositions comprising a compound provided herein, or a pharmaceutically acceptable salt thereof, and at least one pharmaceutically acceptable carrier. The present application further provides methods of modulating the TRPML channel in a subject, the method comprising administering to the subject a therapeutically effective amount of a compound provided herein, or a pharmaceutically acceptable salt thereof.

For example, the disease or disorder can be a ciliopathy (e.g., polycystic kidney disease).

Ciliopathies include, but are not limited to, polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy. Ellis van Creveld syndrome, Sensenbrenner syndrome, and primary ciliary dyskinesia.

### Pharmaceutical Compositions

Pharmaceutical compositions containing compounds described herein such as a compound of Formula (III) or pharmaceutically acceptable salt thereof can be used to treat or ameliorate a disorder described herein.

The amount and concentration of compounds of the disclosure in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

While it is possible for a compound disclosed herein to be administered alone, it is preferable to administer the compound as a pharmaceutical formulation, where the compound is combined with one or more pharmaceutically acceptable diluents, excipients or carriers. The compounds disclosed herein may be formulated for administration in any convenient way for use in human or veterinary medicine. In certain embodiments, the compound included in the pharmaceutical preparation may be active itself, or may be a prodrug, e.g., capable of being converted to an active compound in a physiological setting.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Examples of pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) tale; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins such as Captisol®; and (22) other non-toxic compatible substances employed in pharmaceutical formulations.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Solid dosage forms (e.g., capsules, tablets, pills, dragees, powders, granules and the like) can include one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents.

Liquid dosage forms can include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, tale and zinc oxide, or mixtures thereof.

Powders and sprays can contain, in addition to an active compound, excipients such as lactose, tale, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

The tablets, and other solid dosage forms of the pharmaceutical compositions disclosed herein, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The formulations disclosed herein can be delivered via a device. Exemplary devices include, but are not limited to, a catheter, wire, stent, or other intraluminal device. Further exemplary delivery devices also include a patch, bandage, mouthguard, or dental apparatus. Transdermal patches have the added advantage of providing controlled delivery of a compound disclosed herein to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, drops, solutions and the like, are also contemplated as being within the scope of this disclosure.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds disclosed herein are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The formulations can be administered topically, orally, transdermally, rectally, vaginally, parenterally, intranasally, intrapulmonary, intraocularly, intravenously, intramuscularly, intraarterially, intrathecally, intracapsularly, intraorbitally, intracardiacly, intradermally, intraperitoneally, transtracheally, subcutaneously, subcuticularly, intraarticularly, subcapsularly, subarachnoidly, intraspinally, intrasternally or by inhalation.

### Dosages

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound disclosed herein employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In general, a suitable daily dose of a compound of the disclosure will be that amount of the compound that is the lowest dose effective to produce a therapeutic effect. Such an effective dose will generally depend upon the factors described above. Generally, intravenous, intracerebroventricular, intrathecal and subcutaneous doses of the compounds of this disclosure for a patient will range from about 0.0001 to about 100 mg per kilogram of body weight per day. For example, the dose can be 1-50, 1.25, or 5.10 mg/kg.

If desired, the effective daily dose of the active compound may be administered as two, three, four, five, six or more sub-doses administered separately at appropriate intervals throughout the day, optionally, in unit dosage forms.

### Methods of Treatment

The compounds described herein can be used to treat or prevent a disorder described herein. For example, compounds are provided herein for the prevention, treatment, or alleviating symptoms of a disease or condition associated with the TRPML channel. Compounds useful for modulating TRPML ion channels, including compounds of the disclosure (e.g., compounds of Formula (III), or compounds disclosed in references incorporated herein), or pharmaceutical compositions containing one or more of the compounds, can be administered to treat disorders, conditions, or diseases described herein such as those treatable by modulation of the TRPML ion channel.

In an aspect, provided herein is a method of treating a ciliopathy, the method comprising administering to a patient in need thereof a therapeutically effective amount of a compound or pharmaceutical composition thereof, wherein the compound modulates a TRPML ion channel.

In some embodiments, the compound activates the TRPML ion channel. In some embodiments the TRPML ion channel is TRPML1.

In some embodiments, the compound achieves a maximal current obtained with 30 μM ML-SA1 in a patch clamp assay for TRPML1 which is at least 10 fold the maximal current achieved for any other TRPML channel.

In some embodiments, the ciliopathy is selected from the group consisting of polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy, Ellis van Creveld syndrome, Sensenbrenner syndrome, and primary ciliary dyskinesia.

In some embodiments, the ciliopathy is polycystic kidney disease or pancreatic cysts associated with autosomal dominant polycystic kidney disease. In some embodiments, the ciliopathy is autosomal dominant polycystic kidney disease, autosomal recessive polycystic kidney disease, or pancreatic cysts associated with autosomal dominant polycystic kidney disease. In some embodiments, the ciliopathy is autosomal dominant polycystic kidney disease.

In some embodiments, the method further comprising use of a second therapeutic agent.

In some embodiments, the second therapeutic agent is selected from the group consisting of an mTOR inhibitor, V2 receptor antagonist, tyrosine kinase inhibitor, somatostatin analog, glucosylceramide synthase inhibitor, microRNA-17 inhibitor, siRNA against p53, KEAP1-Nrf2 activator, xanthine oxidase inhibitor, PPARγ agonist, metformin, and beta hydroxybutyrate.

In some embodiments, the second therapeutic agent is selected from the group consisting of tolvaptan, lixivaptan, mozavaptan, satavaptan, sirolimus, tacrolimus, everolimus, bosutinib, tesavatinib, imatinib, gefitinib, erlotinib, dasatinib, octreotide, pasireotide, venglustat, eliglustat, miglustat, microRNA-17 inhibitor, bardoxolone methyl, allopurinol, oxypurinol, pioglitazone, rosiglitazone, lobeglitazone, metformin, and beta hydroxybutyrate.

In some embodiments, the second therapeutic agent is selected from the group consisting of an immunomodulator, a calcineurin inhibitor, a renin angiotensin aldosterone system inhibitor, an antiproliferative agent, an alkylating agent, a corticosteroid, an angiotensin converting enzyme inhibitor, an adrenocorticotropic hormone stimulant, an angiotensin receptor blocker, a sodium-glucose transport protein 2 inhibitor, a dual sodium-glucose transport protein 1/2 inhibitor, a nuclear Factor-1 (erythroid-derived 2)-like 2 agonist, a chemokine receptor 2 inhibitor, a chemokine receptor 5 inhibitor, an endothelin 1 receptor antagonist, a beta blocker, a mineralocorticoid receptor antagonist, a loop or thiazide diuretic, a calcium channel blocker, a statin, a short-intermediate or long-acting insulin, a dipeptidyl peptidase 4 inhibitor, a glucagon-like peptide 1 receptor agonist, a sulfonylurea, an apoptosis signal-regulating kinase-1, a chymase inhibitor, a selective gly cation inhibitor, a renin inhibitor, an interleukin-33 inhibitor, a farnesoid X receptor agonist, a soluble guanylate cyclase stimulator, a thromboxane receptor antagonist, a xanthine oxidase inhibitor, an erythropoietin receptor agonist, a cannabinoid receptor type 1 inverse agonist, a NADPH oxidase inhibitor, an anti-vascular endothelial growth factor B, an anti-fibrotic agent, a neprilysin inhibitor, a dual CD80/CD86 inhibitor, a CD40 antagonist, a cellular cholesterol and lipid blocker, a PDGFR antagonist, a Slit guidance ligand 2, an APOL1 inhibitor, an Nrl2 activator/NF-kB inhibitor, a somatostatin receptor agonist, a PPAR gamma agonist, a AMP activated protein kinase stimulator, a tyrosine kinase inhibitor, a glucosylceramide synthase inhibitor, an arginine vasopressin receptor 2 antagonist, a xanthine oxidase inhibitor, a vasopressin receptor 2 antagonist, anti-amyloid beta antibodies, anti-Tau antibodies, anti-synuclein antibodies, dopamine precursors (e.g. L-DOPA), dopamine agonists (e.g. bromocriptine, cabergoline, pergolide, pramipexole and apomorphine), MAO-B inhibitors (e.g. rasagiline and selegiline), anticholinergics (e.g. orphenadrine, procyclidine and trihexyphenidyl), enhancers of b-glucocerebrosidase activity (e.g. ambroxol and afegostat), amantadine, and agents capable of treating Alzheimer's (e.g., acetylcholinesterase inhibitors such as tacrine, rivastigmine, galantamine, donepezil, and memantine).

In some embodiments, the second therapeutic agent is selected from the group consisting of COX inhibitors including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam; treatments for pulmonary hypertension including prostanoids (epoprostenol, iloprost, and treprostinil), endothelin receptor antagonists (bosentan, ambrisentan, and macitentan), phosphodiesterase-5 inhibitors (sildenafil and tadalafil), and sGC stimulators (riociguat); rho-kinase inhibitors, such as Y-27632, fasudil, and H-1152P; epoprostenol derivatives, such as prostacyclin, treprostinil, beraprost, and iloprost; serotonin blockers, such as sarpogrelate; endothelin receptor antagonists, such as besentan, sitaxsentan, ambrisentan, and TBC3711: PDE inhibitors, such as sildenafil, tadalafil, udenafil, and vardenafil; calcium channel blockers, such as amlodipine, bepridil, clentiazem, diltiazem, fendiline, gallopamil, mibefradil, prenylamine, semotiadil, terodiline, verapamil, aranidipine, bamidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, and perhexiline; tyrosine kinase inhibitors, such as imatinib; inhaled nitric oxide and nitric oxide-donating agents, such as inhaled nitrite: IκB inhibitors, such as IMD 1041; prostacyclin receptor agonists, such as selexipag: stimulators of hematopoiesis, such as TXA 127 (angiotensin (1-7)), darbepoetin alfa, erythropoetin, and epoetin alfa; anticoagulants and platelet-inhibiting agents; and diuretics; dietary and nutritional supplements such as acetyl-L-carnitine, octacosanol, evening primrose oil, vitamin B6, tyrosine, phenylalanine, vitamin C, L-dopa; immunosupressants (for transplants and autoimmune-related RKD); anti-hypertensive drugs (for high blood pressure-related RKD, e.g., angiotensin-converting enzyme inhibitors and angiotensin receptor blockers): insulin (for diabetic RKD); lipid/cholesterol-lowering agents (e.g., HMG-CoA reductase inhibitors such as atorvastatin or simvastatin); and treatments for hyperphosphatemia or hyperparathyroidism associated with CKD (e.g., sevelamer acetate, cinacalcet).

### Disease and Injury Models

Compounds that modulate the TRPML channel may be useful in the prophylaxis and treatment of any of the foregoing injuries, diseases, disorders, or conditions. In addition to in vitro assays of the activity of these compounds, their efficacy can be readily tested in one or more animal models.

### EXAMPLES

### Abbreviations

The following abbreviations are used herein.

| Abbreviation | Meaning |
|---|---|
| DCM | dichloromethane |
| DIC | N,N′-methanediylidenedipropan-2-amine |
| DIPEA | Diisopropylethylamine |
| DMF | N,N-dimethylformamide |
| DMSO | Dimethylsulfoxide |
| EA | Ethyl acetate |
| EGTA | Ethylene glycol-bis(β-aminoethyl ether)-N,N,N′,N′- |
| | tetraacetic acid |
| GTP | Guanosine-5′-triphospphate |
| HEPES | 4-(2-hydroxyethyl)-1-piperazineethanesulfonic acid |
| ¹H NMR | proton nuclear magnetic resonance |
| HPLC | High performance liquid chromatography |
| LCMS | Liquid chromatography mass spectrometry |
| Ms | Methanesulfonyl |
| PE | Petroleum ether |
| Py | Pyridine |
| rt | Room temperature |
| SEM | 2-(Trimethylsilyl)ethoxymethyl |
| TBAF | Tetrabutylammonium fluoride |
| TBAI | Tetrabutylammonium iodide |
| TBDPS | Tert-butyl-diphenylsilyl |
| TBS | Tert-butyl-dimethylsilyl |
| TEA | Triethylamine |
| THF | Tetrahydrofuran |
| Tol | Toluene |
| Ts | p-toluenesulfonyl |

### Example 1. N¹-(2-((1-(4-chloro-2-fluorophenyl)pyrrolidin-3-yl)(methyl)amino)phenyl)-N⁴,N⁴-dimethylbenzene-1,4-disulfonamide (101)

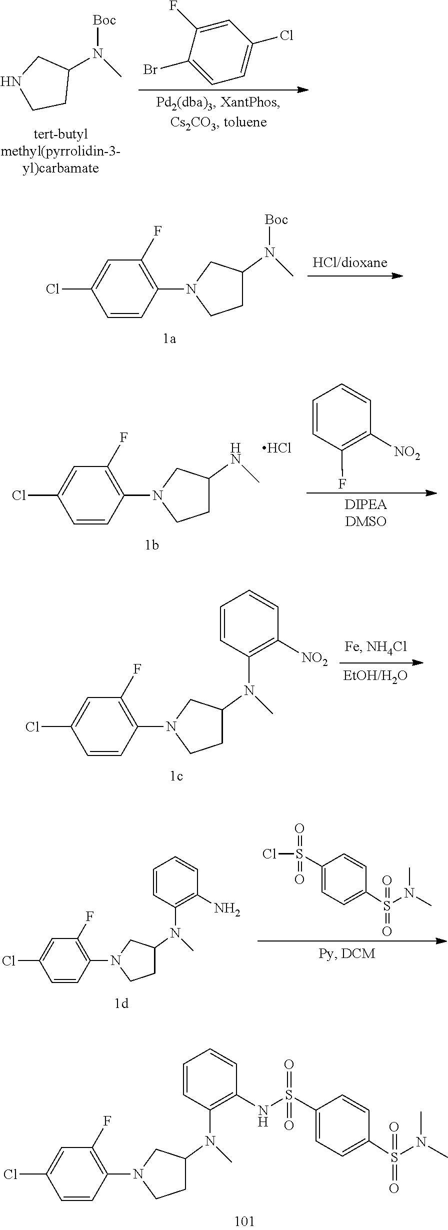

### Compound 1a. tert-butyl (1-(4-chloro-2-fluorophenyl)pyrrolidin-3-yl)(methyl)carbamate

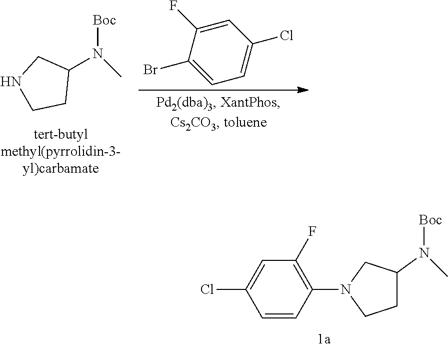

To a mixture of 1-bromo-4-chloro-2-fluorobenzene (1.00 g, 4.77 mmol), tert-butyl methyl(pyrrolidin-3-yl)carbamate (1.05 g, 5.25 mmol), Xantphos (549 mg, 0.95 mmol) and CS₂CO₃ (3.88 g, 11.9 mmol) in toluene (20 mL) was added Pd₂(dba)₃ (440 mg, 0.48 mmol). The mixture was degassed and back-filled with N₂ for three times and stirred at 100° C. under N₂ for 16 h. The resulting mixture was cooled to rt and filtered through a pad of celite. The filtrate was concentrated and purified by silica gel chromatography (PE:EtOAc=10:1, v/v) to give tert-butyl (1-(4-chloro-2-fluorophenyl)pyrrolidin-3-yl)(methyl)carbamate (comp. 1a, 1.5 g, 96%) as a colorless oil. LC/MS ESI (m/z): 329 (M+H)⁺.

### Compound 1b. 1-(4-chloro-2-fluorophenyl)-N-methylpyrrolidin-3-amine hydrochloride

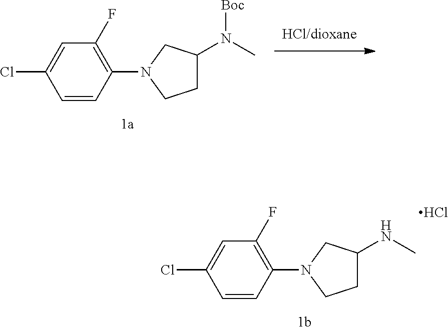 [SMILES: CN(C(=O)OC(C)(C)C)C1CCN(c2ccc(Cl)cc2F)C1 ; CNC1CCN(c2ccc(Cl)cc2F)C1 ; Cl]

To a solution of tert-butyl (1-(4-chloro-2-fluorophenyl)pyrrolidin-3-yl)(methyl)carbamate (1.5 g, 4.57 mmol) in dioxane (15 mL) was added HCl/dioxane (8 mL, 4 M). And the mixture was stirred at 25° C. for 3 h. The resulting mixture was concentrated to give crude 1-(4-chloro-2-fluorophenyl)-N-methylpyrrolidin-3-amine hydrochloride (comp. 1b, 1.2 g, 99%) as a white solid, which was used directly in the next step without further purification. LC/MS ESI (m/z): 229 (M+H)⁺.

### Compound 1c. 1-(4-chloro-2-fluorophenyl)-N-methyl-N-(2-nitrophenyl)pyrrolidin-3-amine

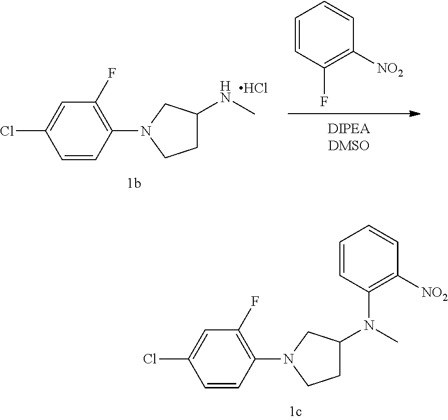 [SMILES: CNC1CCN(c2ccc(Cl)cc2F)C1 ; Cl ; O=[N+]([O-])c1ccccc1F ; CN(c1ccccc1[N+](=O)[O-])C1CCN(c2ccc(Cl)cc2F)C1]

A mixture of 1-(4-chloro-2-fluorophenyl)-N-methylpyrrolidin-3-amine hydrochloride (500 mg, 1.89 mmol), 1-fluoro-2-nitrobenzene (267 mg, 1.89 mmol) and DIPEA (732 mg, 5.67 mmol) in DMSO (8 mL) was stirred at 100° C. for 16 h. Then the mixture was cooled down, poured into water and extracted with ethyl acetate twice. The combined organic layers were dried over anhydrous Na₂SO₄ and filtered. The filtrate was concentrated to dryness and purified by silica gel column chromatography (PE:EtOAc=10:1, v/v) to give 1-(4-chloro-2-fluorophenyl)-N-methyl-N-(2-nitrophenyl)-pyrrolidin-3-amine (comp. 1c, 469 mg, 71%) as a yellow oil. LC/MS ESI (m/z): 350 (M+H)⁺.

### Compound 1d. N¹-(1-(4-chloro-2-fluorophenyl)pyrrolidin-3-yl)-N¹-methylbenzene-1,2-diamine

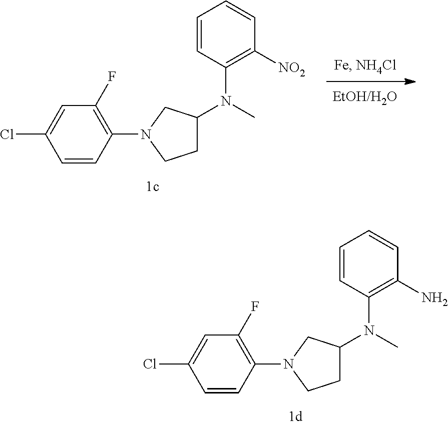 [SMILES: CN(c1ccccc1[N+](=O)[O-])C1CCN(c2ccc(Cl)cc2F)C1 ; CN(c1ccccc1N)C1CCN(c2ccc(Cl)cc2F)C1]

To a solution of 1-(4-chloro-2-fluorophenyl)-N-methyl-N-(2-nitrophenyl)pyrrolidin-3-amine (469 mg, 1.34 mmol) and NH₄Cl (214 mg, 4.02 mmol) in EtOH (8 mL) and water (2 mL) was added reductive iron powder (225 mg, 4.02 mmol) at rt. The mixture was stirred at 85° C. for 2 h. The resulting mixture was filtered through a pad of celite and washed with EtOH. The filtrate was concentrated and purified by silica gel column chromatography (PE:EtOAc=5:1, v/v) to give N¹-(1-(4-chloro-2-fluorophenyl)pyrrolidin-3-yl)-N¹-methylbenzene-1,2-diamine (comp. 1d, 400 mg, 94%) as a white solid. LC/MS ESI (m/z): 320 (M+H)⁺.

### Compound 101. N¹-(2-((1-(4-chloro-2-fluorophenyl)pyrrolidin-3-yl)(methyl)amino)phenyl)-N⁴,N⁴-dimethylbenzene-1,4-disulfonamide

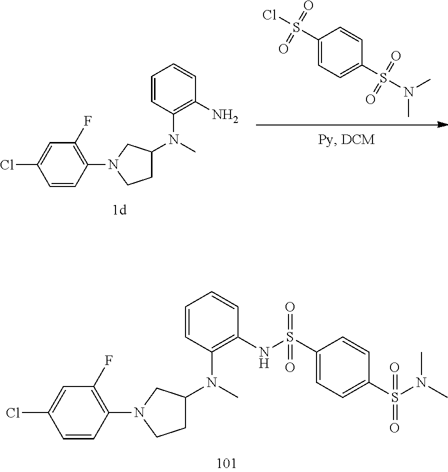 [SMILES: CN(c1ccccc1N)C1CCN(c2ccc(Cl)cc2F)C1 ; CN(C)S(=O)(=O)c1ccc(S(=O)(=O)Cl)cc1 ; CN(c1ccccc1NS(=O)(=O)c1ccc(S(=O)(=O)N(C)C)cc1)C1CCN(c2ccc(Cl)cc2F)C1]

To a solution of N¹-(1-(4-chloro-2-fluorophenyl)pyrrolidin-3-yl)-N¹-methylbenzene-1,2-diamine (200 mg, 0.63 mmol) and pyridine (100 mg, 1.26 mmol) in DCM (3 mL) was added 4-(N,N-dimethylsulfamoyl)benzenesulfonyl chloride (213 mg, 0.75 mmol) at 0° C. The mixture was stirred at 40° C. for 16 h. The resulting solution was diluted with water and extracted with DCM twice. The combined organic layers were dried over anhydrous Na₂SO₄, filtered and concentrated in vacuo. The residue was purified by prep-HPLC to give N¹-(2-((1-(4-chloro-2-fluorophenyl)pyrrolidin-3-yl)(methyl)amino)phenyl)-N⁴,N⁴-dimethylbenzene-1,4-disulfonamide (comp. 101, 55 mg, 15%) as a white solid. LC/MS ESI (m/z): 567 (M+H)⁺.

¹H NMR (400 MHz, CDCl₃) δ 8.51 (s, 1H), 7.98 (d, J=8.4 Hz, 2H), 7.79 (t, J=9.8 Hz, 2H), 7.55 (dd, J=12.0, 5.1 Hz, 1H), 7.17 (dd, J=13.7, 7.7 Hz, 2H), 7.08 (dt, J=7.6, 3.8 Hz, 1H), 7.04-6.96 (m, 2H), 6.55 (t, J=9.0 Hz, 1H), 3.70-3.57 (m, 1H), 3.38-3.29 (m, 2H), 3.17-3.08 (m, 1H), 2.70 (s, 6H), 2.39 (s, 3H), 2.02-1.93 (m, 1H), 1.77-1.64 (m, 1H).

### Example 2. Synthesis of N¹-(2-(2-(4-chloro-2-fluorophenyl)-2,6-diazaspiro[3.4]octan-6-yl)phenyl)-N⁴,N⁴-dimethylbenzene-1,4-disulfonamide (Compound 102)

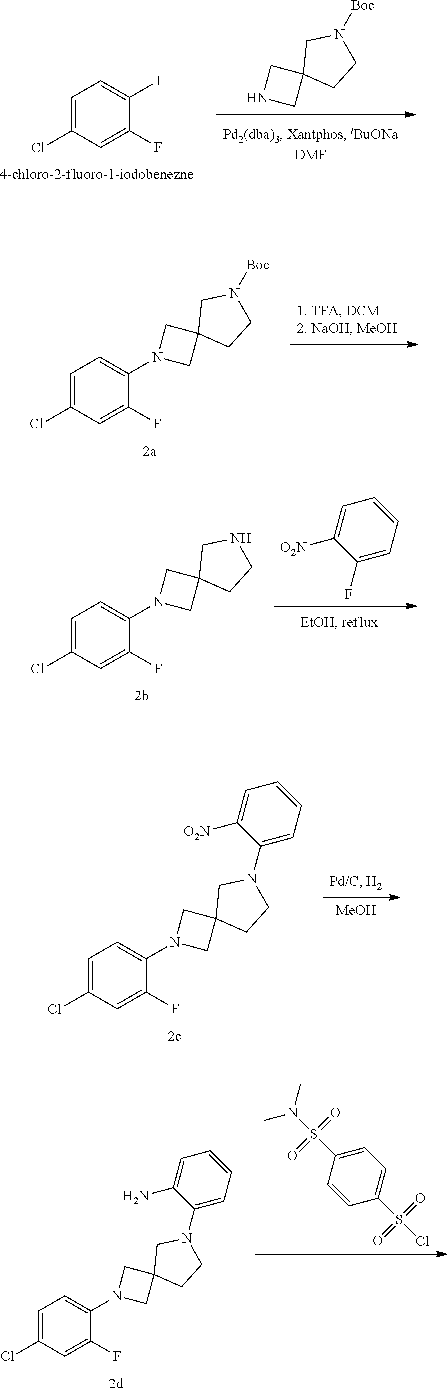

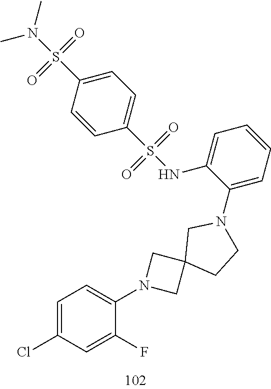 [SMILES: CN(C)S(=O)(=O)c1ccc(S(=O)(=O)Nc2ccccc2N2CCC3(CN(c4ccc(Cl)cc4F)C3)C2)cc1]

### Compound 2a. tert-butyl 2-(4-chloro-2-fluorophenyl)-2,6-diazaspiro[3.4]octane-6-carboxylate

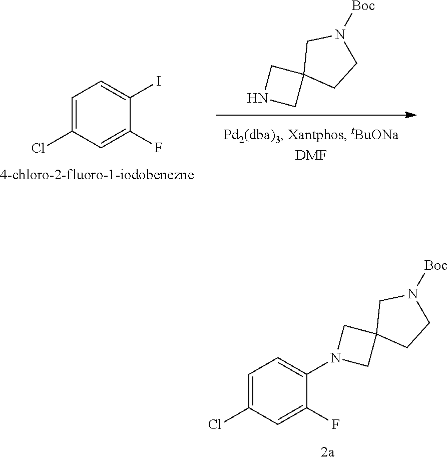 [SMILES: Fc1cc(Cl)ccc1I ; CC(C)(C)OC(=O)N1CCC2(CNC2)C1 ; CC(C)(C)OC(=O)N1CCC2(C1)CN(c1ccc(Cl)cc1F)C2]

An oven-dried round-bottom flask was purged with N₂ and charged with Pd₂(dba)₃ (46 mg, 0.05 mmol), Xantphos (29 mg, 0.05 mmol), sodium tert-butoxide (150 mg, 1.50 mmol) and 4-chloro-2-fluoro-1-iodobenzene (256 mg, 1.00 mmol). The flask was purged with N₂. Then DMF (2.50 mL) and tert-butyl 2,6-diazaspiro[3.4]octane-6-carboxylate (212 mg, 1.00 mmol) were added through a rubber septum. The septum was removed. The mixture was stirred at room temperature for 4 h and then diluted with EtOAc, filtered through celite and concentrated in vacuo. The crude material was purified by flash chromatography on silica gel (PE:EA=100:1 to 5:1, v/v) to afford tert-butyl 2-(4-chloro-2-fluorophenyl)-2,6-diazaspiro[3.4]octane-6-carboxylate (comp. 2a, 283 mg, 83%) as a yellow solid. LC/MS ESI (m/z): 341 (M+H)⁺.

### Compound 2b. 2-(4-chloro-2-fluorophenyl)-2,6-diazaspiro[3.4]octane

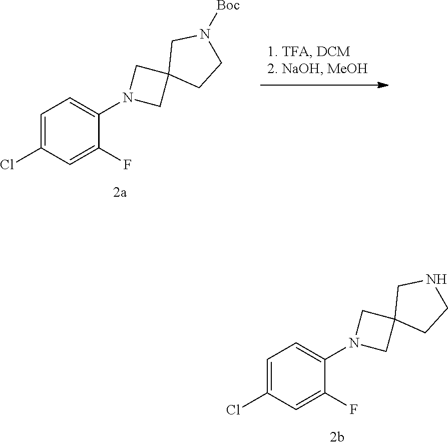 [SMILES: CC(C)(C)OC(=O)N1CCC2(C1)CN(c1ccc(Cl)cc1F)C2 ; Fc1cc(Cl)ccc1N1CC2(CCNC2)C1]

To a solution of tert-butyl 2-(4-chloro-2-fluorophenyl)-2,6-diazaspiro[3.4]octane-6-carboxylate (283 mg, 0.83 mmol) in DCM (2 mL) was added TFA (1 mL) at rt. The reaction was stirred at rt for 3 h and was concentrated and diluted with MeOH (10 mL). NaOH (4.15 mL, 2 M) was added and the mixture stirred at rt for 30 min. Then the mixture was concentrated and extracted with DCM (20 mL×3). The organic layer was dried over anhydrous Na₂SO₄ to give 2-(4-chloro-2-fluorophenyl)-2,6-diazaspiro[3.4]octane (comp. 2b, 195 mg, 97%) as a yellow oil which was used in the next step without further purification. LC/MS ESI (m/z): 241 (M+H)⁺.

### Compound 2c. 2-(4-chloro-2-fluorophenyl)-6-(2-nitrophenyl)-2,6-diazaspiro[3.4]octane

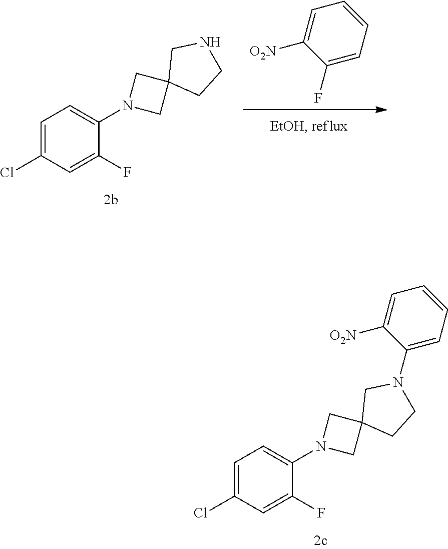 [SMILES: Fc1cc(Cl)ccc1N1CC2(CCNC2)C1 ; O=[N+]([O-])c1ccccc1F ; O=[N+]([O-])c1ccccc1N1CCC2(CN(c3ccc(Cl)cc3F)C2)C1]

In a sealed tube, a solution of 2-(4-chloro-2-fluorophenyl)-2,6-diazaspiro[3.4]octane (195 mg, 0.81 mmol), triethylamine (0.11 mL, 0.81 mmol) and 1-fluoro-2-nitrobenzene (0.09 mL, 0.81 mmol) in ethanol (5 mL) was stirred at 100° C. for 3 h. The mixture was concentrated under vacuum. The residue was purified by column chromatography (silica gel, 0˜5% MeOH in DCM) to give 2-(4-chloro-2-fluorophenyl)-6-(2-nitrophenyl)-2,6-diazaspiro[3.4]octane (comp. 2c, 290 mg, 98%) which was used in the next step without further purification. LC/MS ESI (m/z): 362 (M+H)⁺.

### Compound 2d. 2-(2-(4-chloro-2-fluorophenyl)-2,6-diazaspiro[3.4]octan-6-yl)aniline

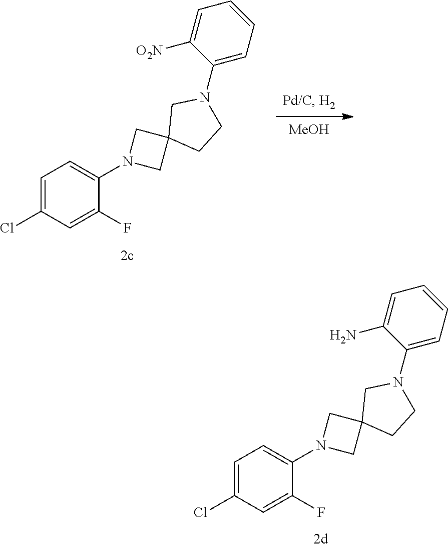 [SMILES: "MeOH" ; O=[N+]([O-])c1ccccc1N1CCC2(CN(c3ccc(Cl)cc3F)C2)C1 ; Nc1ccccc1N1CCC2(C1)CN(c1ccc(Cl)cc1F)C2]

A solution of 2-(4-chloro-2-fluorophenyl)-6-(2-nitrophenyl)-2,6-diazaspiro[3.4]octane (290 mg, 0.80 mmol) and Pd/C (60 mg, 50% water wet) in MeOH (10 mL) was stirred under H₂ at rt overnight. The organic layer was filtered and concentrated, dried over anhydrous Na₂SO₄ and concentrated. The residue was purified by column chromatography (silica gel, 0˜5% EtOAc in petroleum ether) to give 2-[2-(4-chloro-2-fluorophenyl)-2,6-diazaspiro[3.4]octan-6-yl]aniline (comp. 2d, 190 mg, 71%). LC/MS ESI (m/z): 332 (M+H)⁺.

### Compound 102. N¹-(2-(2-(4-chloro-2-fluorophenyl)-2,6-diazaspiro[3.4]octan-6-yl)phenyl)-N⁴,N⁴-dimethylbenzene-1,4-disulfonamide

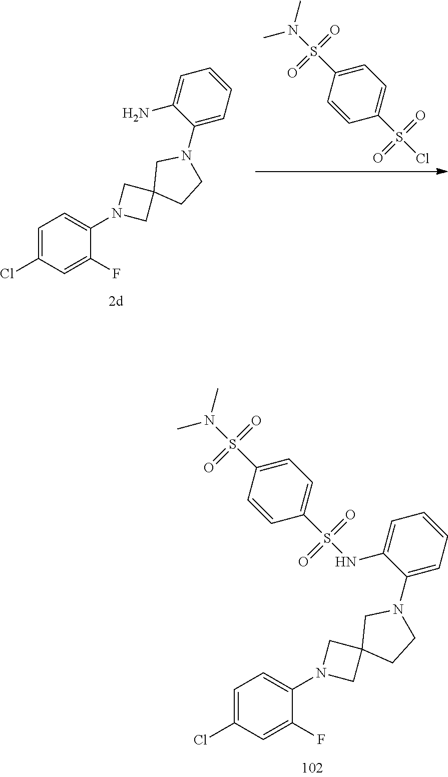 [SMILES: Nc1ccccc1N1CCC2(C1)CN(c1ccc(Cl)cc1F)C2 ; CN(C)S(=O)(=O)c1ccc(S(=O)(=O)Cl)cc1 ; CN(C)S(=O)(=O)c1ccc(S(=O)(=O)Nc2ccccc2N2CCC3(CN(c4ccc(Cl)cc4F)C3)C2)cc1]

To a solution of 2-[2-(4-chloro-2-fluorophenyl)-2,6-diazaspiro[3.4]octan-6-yl]aniline (140 mg, 0.42 mmol) and pyridine (0.10 mL, 1.27 mmol) in DCM (10 mL) was added 4-(N,N-dimethylsulfamoyl)benzenesulfonyl chloride (120 mg, 0.42 mmol) very slowly in ice-bath. Then the mixture was warmed to rt and stirred overnight. Then the mixture was concentrated and purified by column chromatography (silica gel, 10%˜40% EtOAc in petroleum ether) to give N⁴-{2-[2-(4-chloro-2-fluorophenyl)-2,6-diazaspiro[3.4]octan-6-yl]phenyl)-N¹,N¹-dimethylbenzene-1,4-disulfonamide (comp. 102, 110 mg, 45%) as a yellow foam. LC/MS ESI (m/z): 579 (M+H)⁺. ¹H NMR (400 MHz, DMSO-d₆) δ 9.59 (s, 1H), 7.98-7.86 (m, 4H), 7.23 (dd, J=12.3, 2.4 Hz, 1H), 7.12-7.04 (m, 2H), 6.80-6.72 (m, 1H), 6.64-6.50 (m, 3H), 3.89-3.79 (m, 4H), 3.44 (s, 2H), 3.28 (t, J=6.8 Hz, 2H), 2.63 (s, 6H), 2.06 (t, J=6.8 Hz, 2H).

### Example 3. Synthesis of N¹-(2-(7-(4-chloro-2-fluorophenyl)-2,7-diazaspiro[4.4]nonan-2-yl)phenyl)-N⁴,N⁴-dimethylbenzene-1,4-disulfonamide (Compound 103)

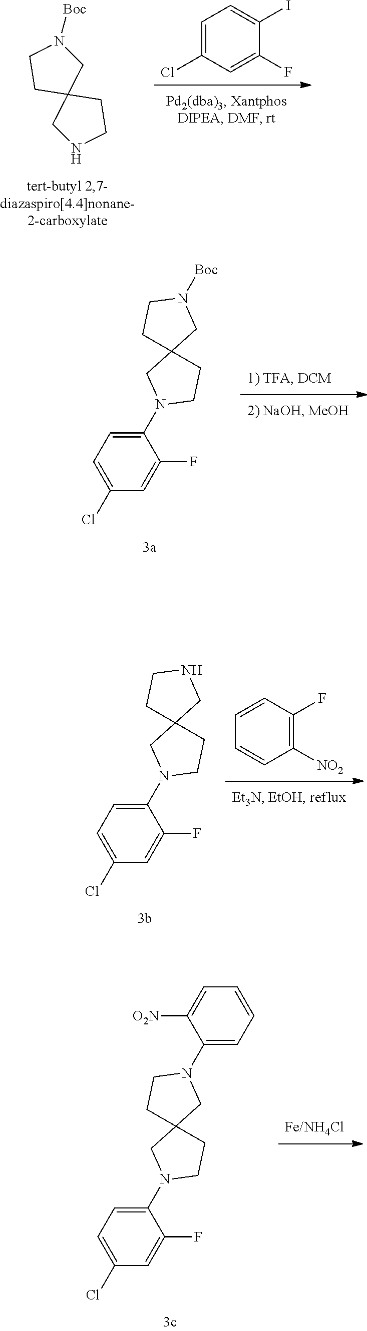

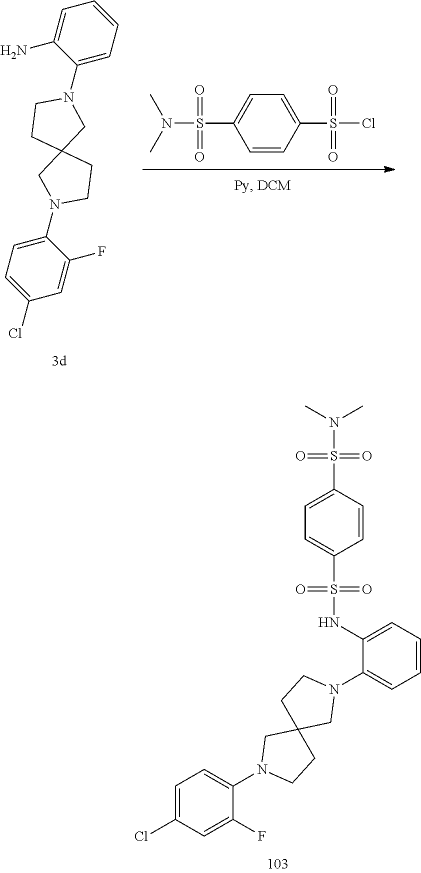 [SMILES: Nc1ccccc1N1CCC2(CCN(c3ccc(Cl)cc3F)C2)C1 ; CN(C)S(=O)(=O)c1ccc(S(=O)(=O)Cl)cc1 ; CN(C)S(=O)(=O)c1ccc(S(=O)(=O)Nc2ccccc2N2CCC3(CCN(c4ccc(Cl)cc4F)C3)C2)cc1]

### Compound 3a. tert-butyl 7-(4-chloro-2-fluorophenyl)-2,7-diazaspiro[4.4]nonane-2-carboxylate

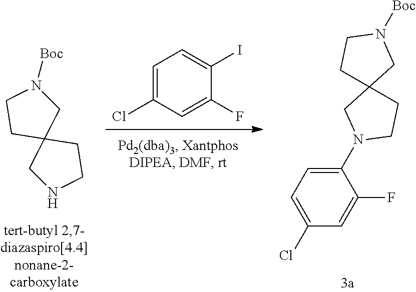 [SMILES: CC(C)(C)OC(=O)N1CCC2(CCNC2)C1 ; Fc1cc(Cl)ccc1I ; CC(C)(C)OC(=O)N1CCC2(CCN(c3ccc(Cl)cc3F)C2)C1]

An oven-dried round-bottom flask was purged with N₂ and charged with tert-butyl 2,7-diazaspiro[4.4]nonane-2-carboxylate (500 mg, 2.21 mmol), Pd₂(dba)₃ (101 mg, 0.11 mmol), Xantphos (64 mg, 0.11 mmol), sodium tert-butoxide (318 mg, 3.31 mmol) and 4-chloro-2-fluoro-1-iodobenzene (567 mg, 2.21 mmol). The mixture was stirred at room temperature for 4 h and then diluted with EtOAc, filtered through celite and concentrated in vacuo. The crude material was purified by flash chromatography on silica gel (PE:EA=50:1 to 10:1, v/v) to afford tert-butyl 7-(4-chloro-2-fluorophenyl)-2,7-diazaspiro[4.4]nonane-2-carboxylate (comp. 3a, 450 mg, 57%) as a yellow oil. LC/MS ESI (m/z): 355 (M+H)⁺.

### Compound 3b. 2-(4-chloro-2-fluorophenyl)-2,7-diazaspiro[4.4]nonane

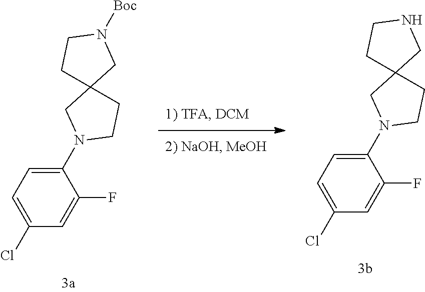 [SMILES: CC(C)(C)OC(=O)N1CCC2(CCN(c3ccc(Cl)cc3F)C2)C1 ; Fc1cc(Cl)ccc1N1CCC2(CCNC2)C1]

To a solution of tert-butyl 7-(4-chloro-2-fluorophenyl)-2,7-diazaspiro[4.4]nonane-2-carboxylate (450 mg, 1.27 mmol) in DCM (3 mL) was added TFA (1.5 mL) at rt. The reaction was stirred at rt for 3 h. The mixture was concentrated and diluted with MeOH (10 mL). NaOH (6.3 mL, 2 M) was added and the mixture was stirred at rt for 30 min. Then the mixture was concentrated and extracted with DCM (20 mL×3). The organic layer was dried over anhydrous Na₂SO₄ and concentrated to give 2-(4-chloro-2-fluorophenyl)-2,7-diazaspiro[4.4]nonane (comp. 3b, 300 mg, 93%) as a yellow oil, which was used for the next step without further purification.

### Compound 3c. 2-(4-chloro-2-fluorophenyl)-7-(2-nitrophenyl)-2,7-diazaspiro[4.4]nonane

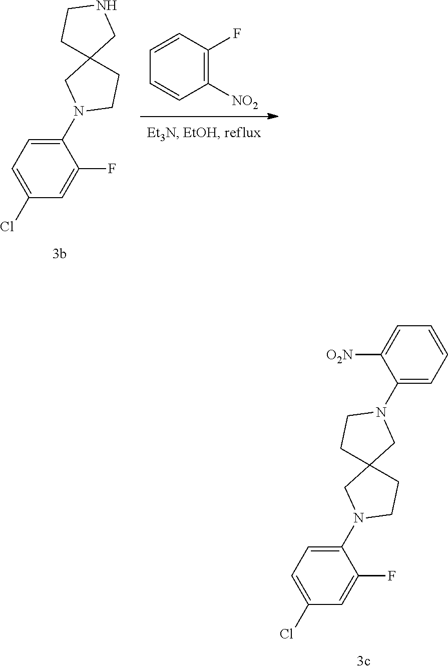

In a sealed tube, a solution of 2-(4-chloro-2-fluorophenyl)-2,7-diazaspiro[4.4]nonane (300 mg, 1.18 mmol), Et₃N (0.33 mL, 2.36 mmol) and 1-fluoro-2-nitrobenzene (0.12 mL, 1.18 mmol) in ethanol (5 mL) was stirred at 80° C. for 2 h. The mixture was concentrated under vacuum. The residue was used for the next step without further purification. Compound 3c, LC/MS ESI (m/z): 376 (M+H)⁺.

### Compound 3d. 2-(7-(4-chloro-2-fluorophenyl)-2,7-diazaspiro[4.4]nonan-2-yl)aniline

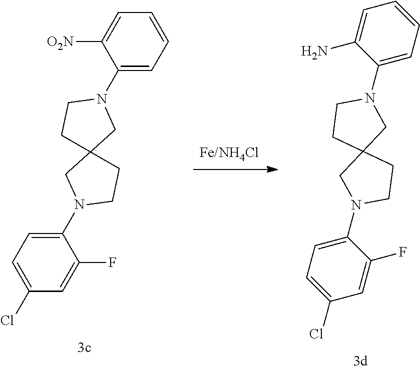 [SMILES: O=[N+]([O-])c1ccccc1N1CCC2(CCN(c3ccc(Cl)cc3F)C2)C1 ; Nc1ccccc1N1CCC2(CCN(c3ccc(Cl)cc3F)C2)C1]

To a solution of 2-(4-chloro-2-fluorophenyl)-7-(2-nitrophenyl)-2,7-diazaspiro[4.4]nonane (160 mg, 0.43 mmol) in MeOH (5 mL) and saturated aqueous NH₄Cl was added Fe powder (50 mg, 0.44 mmol). The mixture was heated to reflux and stirred for 1.5 h. The mixture was cooled to rt and filtered. The filtrate was concentrated and re-dissolved in 10 mL of DCM. The resulting solution was washed with water twice and the organic layer was dried and concentrated to afford crude 2-(7-(4-chloro-2-fluorophenyl)-2,7-diazaspiro[4.4]nonan-2-yl)aniline (comp. 3d, 140 mg, 95%) which was used in the next step directly without further purification. LC/MS ESI (m/z): 346 (M+H)⁺.

### Compound 103. N¹-(2-(7-(4-chloro-2-fluorophenyl)-2,7-diazaspiro[4.4]nonan-2-yl)phenyl)-N⁴,N⁴-dimethylbenzene-1,4-disulfonamide

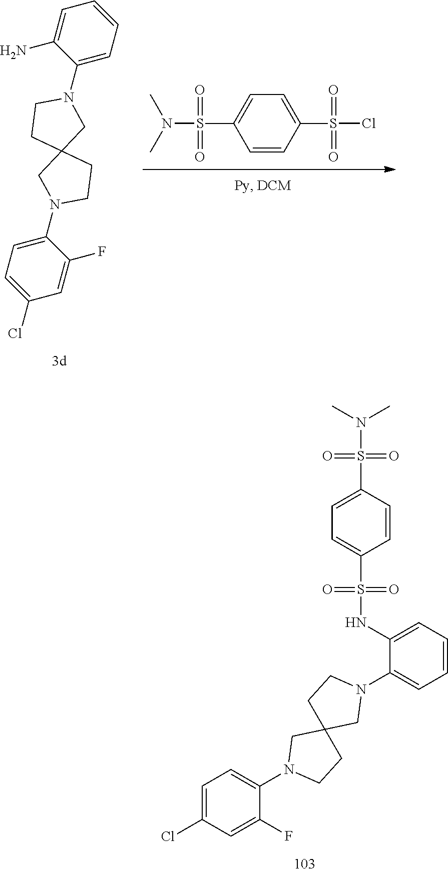 [SMILES: Nc1ccccc1N1CCC2(CCN(c3ccc(Cl)cc3F)C2)C1 ; CN(C)S(=O)(=O)c1ccc(S(=O)(=O)Cl)cc1 ; CN(C)S(=O)(=O)c1ccc(S(=O)(=O)Nc2ccccc2N2CCC3(CCN(c4ccc(Cl)cc4F)C3)C2)cc1]

To a solution of 2-(7-(4-chloro-2-fluorophenyl)-2,7-diazaspiro[4.4]nonan-2-yl)aniline (120 mg, 0.35 mmol) in DCM (5 mL) and pyridine (41 mg, 0.52 mmol) was added 4-(N,N-dimethylsulfamoyl)benzenesulfonyl chloride (108 mg, 0.38 mmol) at 0° C. The mixture was stirred at rt overnight and then filtered. The filtrate was concentrated. The residue was purified by flash chromatography on silica gel (PE:EA=50:1 to 2:3, v/v) to afford N¹-(2-(7-(4-chloro-2-fluorophenyl)-2,7-diazaspiro[4.4]nonan-2-yl)phenyl)-N⁴,N⁴-dimethylbenzene-1,4-disulfonamide (comp. 103, 80 mg, 39%) as a white foam. LC/MS ESI (m/z): 593 (M+H)⁺. ¹H NMR (400 MHz, DMSO-d₆) δ 9.57 (s, 1H), 7.92 (q, J=8.6 Hz, 3H), 7.22 (dd, J=13.6, 2.5 Hz, 1H), 7.14-7.02 (m, 2H), 6.81-6.67 (m, 2H), 6.60-6.44 (m, 2H), 3.48-3.42 (m, 5H), 3.30 (d, J=2.6 Hz, 2H), 3.25-3.22 (m, 2H), 2.64 (s, 6H), 1.95-1.78 (m, 4H).

### Example 4. N¹-(2-(3-((4-chloro-2-fluorophenyl)(methyl)amino)pyrrolidin-1-yl)phenyl)-N⁴,N⁴-dimethylbenzene-1,4-disulfonamide (Compound 104)

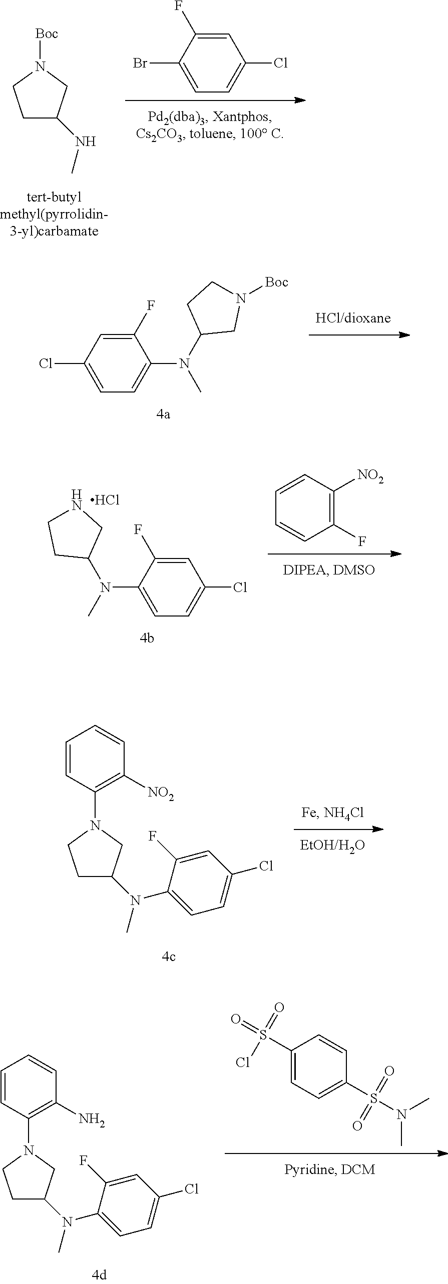

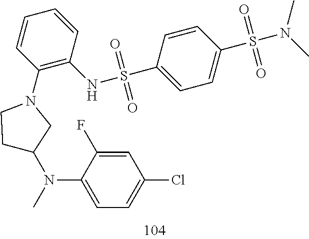 [SMILES: CN(c1ccc(Cl)cc1F)C1CCN(c2ccccc2NS(=O)(=O)c2ccc(S(=O)(=O)N(C)C)cc2)C1]

### Compound 4a. tert-butyl 3-((4-chloro-2-fluorophenyl)(methyl)amino)pyrrolidine-1-carboxylate

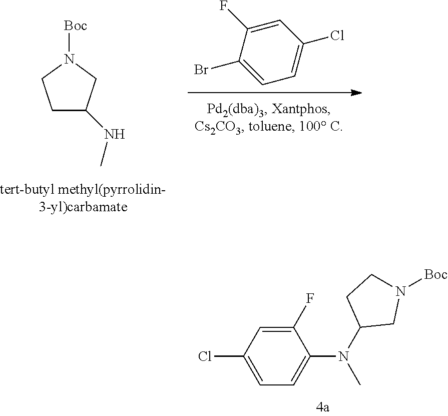 [SMILES: CNC1CCN(C(=O)OC(C)(C)C)C1 ; Fc1cc(Cl)ccc1Br ; CN(c1ccc(Cl)cc1F)C1CCN(C(=O)OC(C)(C)C)C1]

To a mixture of 1-bromo-4-chloro-2-fluorobenzene (1.1 g, 5.3 mmol), tert-butyl methyl(pyrrolidin-3-yl)carbamate (1.17 g, 5.84 mmol), Xantphos (615 mg, 1.06 mmol) and Cs₂CO₃ (5.16 g, 15.9 mmol) in toluene (25 mL) was added Pd₂(dba)₃ (486 mg, 0.53 mmol). The mixture was degassed and back-filled with N₂ for three times and stirred at 100° C. under N₂ atmosphere for 16 h. The resulting mixture was cooled to rt and filtered through a pad of celite. The filtrate was purified by silica gel column chromatography (PE:EtOAc=10:1, v/v) to give tert-butyl 3-((4-chloro-2-fluorophenyl)(methyl)amino)pyrrolidine-1-carboxylate (comp. 4a, 250 mg, 14%) as a colorless oil. LC/MS ESI (m/z): 329 (M+H)⁺.

### Compound 4b. N-(4-chloro-2-fluorophenyl)-N-methylpyrrolidin-3-amine hydrochloride

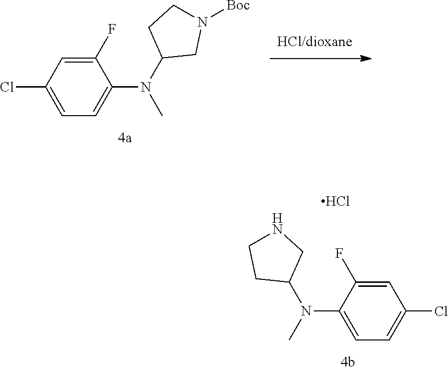 [SMILES: CN(c1ccc(Cl)cc1F)C1CCN(C(=O)OC(C)(C)C)C1 ; Cl ; CN(c1ccc(Cl)cc1F)C1CCNC1]

To a solution of tert-butyl 3-((4-chloro-2-fluorophenyl)(methyl)amino)pyrrolidine-1-carboxylate (250 mg, 0.76 mmol) in dioxane (5 mL) was added HCl/dioxane (5 mL, 4 M) and the mixture was stirred at 25° C. for 3 h. The mixture was concentrated to give crude N-(4-chloro-2-fluorophenyl)-N-methylpyrrolidin-3-amine hydrochloride (220 mg, 96%) as a white solid, which was used directly in the next step without further purification. Compound 4b, LC/MS ESI (m/z): 229 (M+H)⁺.

### Compound 4c. N-(4-chloro-2-fluorophenyl)-N-methyl-1-(2-nitrophenyl)pyrrolidin-3-amine

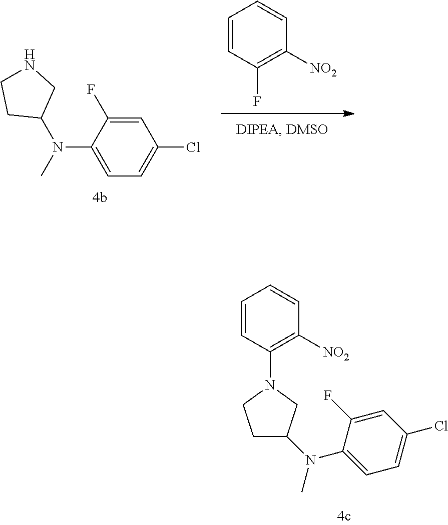 [SMILES: CN(c1ccc(Cl)cc1F)C1CCNC1 ; O=[N+]([O-])c1ccccc1F ; CN(c1ccc(Cl)cc1F)C1CCN(c2ccccc2[N+](=O)[O-])C1]

A mixture of N-(4-chloro-2-fluorophenyl)-N-methylpyrrolidin-3-amine hydrochloride (220 mg, 0.73 mmol), 1-fluoro-2-nitrobenzene (113 mg, 0.80 mmol) and DIPEA (472 mg, 3.67 mmol) in DMSO (5 mL) was stirred at 100° C. for 16 h. Then the mixture was cooled down, poured into water and extracted with ethyl acetate twice. The combined organic layers were dried over anhydrous Na₂SO₄, filtered and concentrated to dryness. The residue was purified by silica gel column chromatography (PE:EtOAc=10:1, v/v) to give N-(4-chloro-2-fluorophenyl)-N-methyl-1-(2-nitrophenyl)pyrrolidin-3-amine (220 mg, 86%) as a yellow oil. Compound 4c, LC/MS ESI (m/z): 350 (M+H)⁺.

### Compound 4d. 1-(2-aminophenyl)-N-(4-chloro-2-fluorophenyl)-N-methylpyrrolidin-3-amine

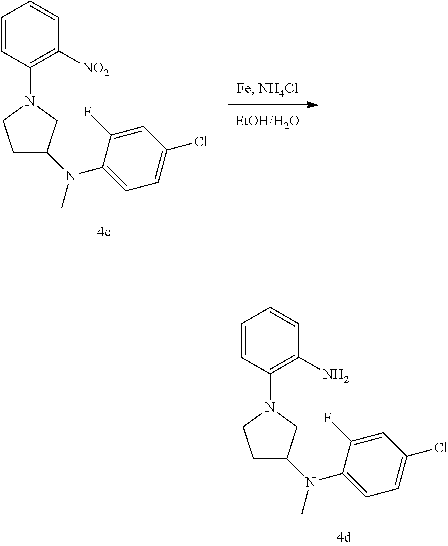 [SMILES: CN(c1ccc(Cl)cc1F)C1CCN(c2ccccc2[N+](=O)[O-])C1 ; CN(c1ccc(Cl)cc1F)C1CCN(c2ccccc2N)C1]

To a solution of N-(4-chloro-2-fluorophenyl)-N-methyl-1-(2-nitrophenyl)pyrrolidin-3-amine (220 mg, 0.63 mmol) and NH₄Cl (214 mg, 6.3 mmol) in EtOH (8 mL) and water (2 mL) was added reductive iron powder (176 mg, 3.15 mmol) and the mixture was stirred at 85° C. for 2 h. The resulting mixture was filtered through a pad of celite and washed with EtOH. The filtrate was concentrated and purified by silica gel column chromatography (PE:EtOAc=5:1, v/v) to give 1-(2-aminophenyl)-N-(4-chloro-2-fluorophenyl)-N-methylpyrrolidin-3-amine (200 mg, 99%) as a white solid. Compound 4d, LC/MS ESI (m/z): 320 (M+H)⁺.

### Compound 104. N¹-(2-(3-((4-chloro-2-fluorophenyl)(methyl)amino)pyrrolidin-1-yl)phenyl)-N⁴,N⁴-dimethylbenzene-1,4-disulfonamide

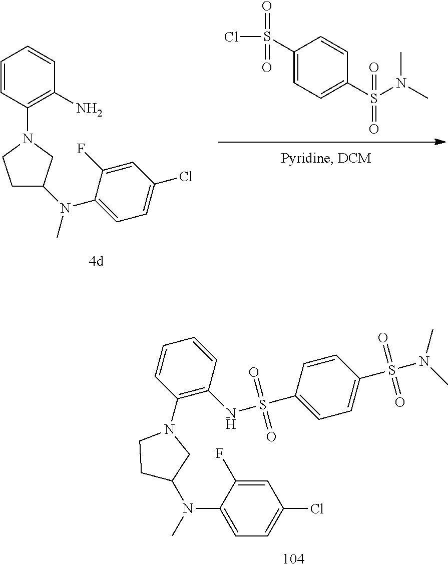 [SMILES: CN(c1ccc(Cl)cc1F)C1CCN(c2ccccc2N)C1 ; CN(C)S(=O)(=O)c1ccc(S(=O)(=O)Cl)cc1 ; CN(c1ccc(Cl)cc1F)C1CCN(c2ccccc2NS(=O)(=O)c2ccc(S(=O)(=O)N(C)C)cc2)C1]

To a solution of 1-(2-aminophenyl)-N-(4-chloro-2-fluorophenyl)-N-methylpyrrolidin-3-amine (200 mg, 0.63 mmol) and pyridine (100 mg, 1.26 mmol) in DCM (3 mL) was added 4-(N,N-dimethylsulfamoyl)benzenesulfonyl chloride (213 mg, 0.75 mmol) at 0° C. The mixture was stirred at 40° C. for 16 h. The resulting solution was diluted with water and extracted with DCM twice. The combined organic layers were dried over anhydrous Na₂SO₄, filtered and concentrated in vacuo. The residue was purified by prep-HPLC to give N¹-(2-(3-((4-chloro-2-fluorophenyl)(methyl)amino)pyrrolidin-1-yl)phenyl)-N⁴,N⁴-dimethylbenzene-1,4-disulfonamide (comp. 104, 60 mg, 17%) as a white solid. LC/MS ESI (m/z): 567 (M+H)⁺. ¹H NMR (400 MHz, CD₃OD) δ 7.93-7.86 (m, 4H), 7.16-7.11 (m, 3H), 7.11-7.08 (m, 1H), 6.99 (dd, J=7.9, 1.5 Hz, 1H), 6.92 (dd, J=8.2, 1.2 Hz, 1H), 6.79-6.75 (m, 1H), 3.97-3.90 (m, 1H), 3.17 (dd, J=9.7, 6.3 Hz, 1H), 3.11 (dt, J=7.7, 5.0 Hz, 3H), 2.76 (s, 3H), 2.68 (s, 6H), 2.06 (dt, J=11.8, 4.8 Hz, 1H), 1.92-1.83 (m, 1H).

### Example 5. N¹-((1S,2S)-2-(4-(4-chloro-2-fluorophenyl)piperazin-1-yl)cyclohexyl)-N⁴,N⁴-dimethylbenzene-1,4-disulfonamide (Compound 105)

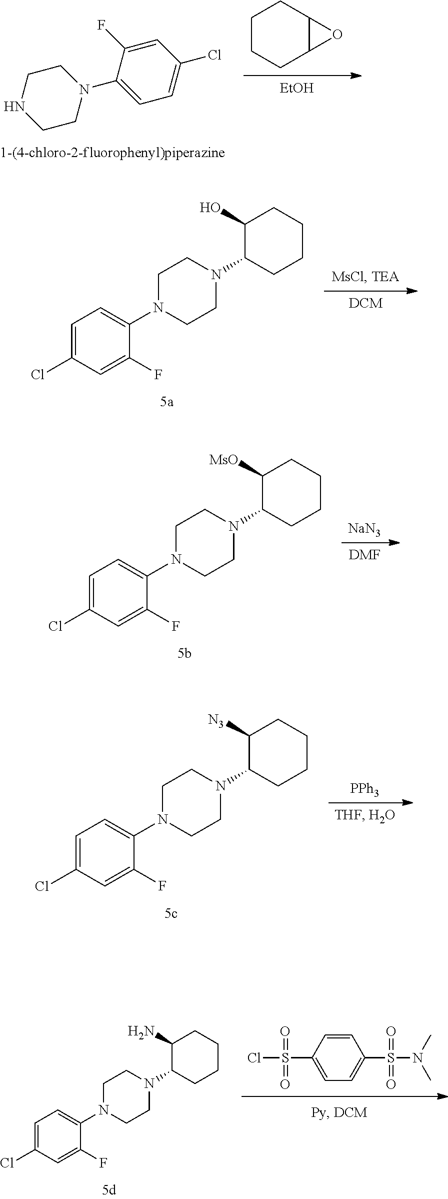

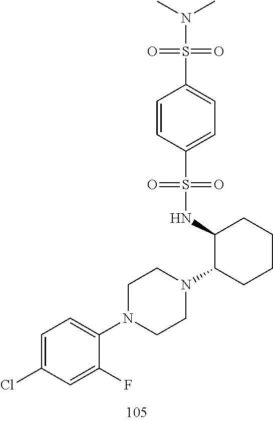 [SMILES: CN(C)S(=O)(=O)c1ccc(S(=O)(=O)N[C@H]2CCCC[C@@H]2N2CCN(c3ccc(Cl)cc3F)CC2)cc1]

### Compound 5a. (1S,2S)-2-(4-(4-chloro-2-fluorophenyl)piperazin-1-yl)cyclohexan-1-ol

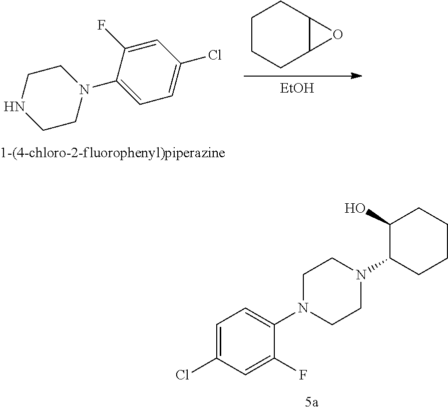

To a solution of 1-(4-chloro-2-fluorophenyl)piperazine (500 mg, 2.34 mmol) in EtOH (15 mL) was added 7-oxabicyclo[4.1.0]heptane (1.2 mL, 23 mmol). The mixture was stirred at reflux overnight. Then the resulting solution was concentrated in vacuo and the residue was purified by silica gel column chromatography to give (1S,2S)-2-(4-(4-chloro-2-fluorophenyl)piperazin-1-yl)cyclohexan-1-ol (comp. 5a, 470 mg, 64%) as a yellow solid, which was used directly in the next step.

### Compound 5b. (1S,2S)-2-(4-(4-chloro-2-fluorophenyl)piperazin-1-yl)cyclohexyl methanesulfonate

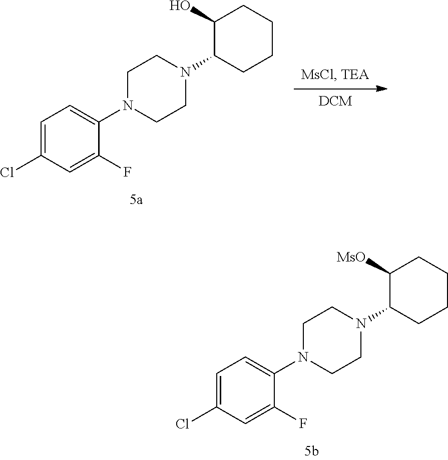

To a mixture of (1S,2S)-2-(4-(4-chloro-2-fluorophenyl)piperazin-1-yl)cyclohexan-1-ol (200 mg, 0.64 mmol) and TEA (0.1 mL) in DCM (10 mL) was added a solution of MsCl (0.23 mL) in DCM (3 mL) at 0° C. and stirred at rt for 1 h. Then the mixture was washed with aq. NaHCO₃ and extracted with DCM (20 mL). The combined organic layers were dried over anhydrous Na₂SO₄ and concentrated in vacuo to give (1S,2S)-2-(4-(4-chloro-2-fluorophenyl)piperazin-1-yl)cyclohexyl methanesulfonate (comp. 5b, 200 mg, 80%) as a yellow oil which was used directly in the next step.

### Compound 5c. 1-((1S,2S)-2-azidocyclohexyl)-4-(4-chloro-2-fluorophenyl)piperazine

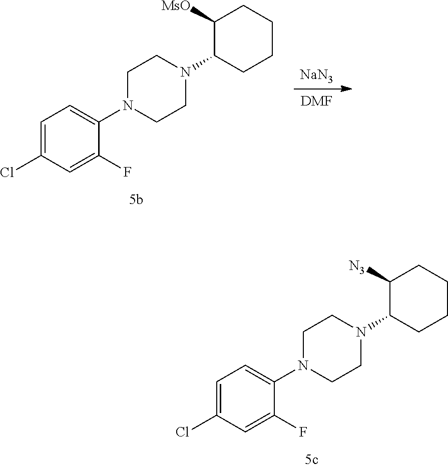

To a solution of (1S,2S)-2-(4-(4-chloro-2-fluorophenyl)piperazin-1-yl)cyclohexyl methanesulfonate (200 mg, 0.513 mmol) in DMF (10 mL) was added NaN₃ (80 mg, 1.2 mmol). The mixture was stirred at rt for 1 h. Then the resulting solution was washed with water (20 mL) and extracted with EtOAc (20 mL). The combined organic layers were dried over anhydrous Na₂SO₄ and concentrated under reduced pressure and purified by silica gel column chromatography to give 1-((1S,2S)-2-azidocyclohexyl)-4-(4-chloro-2-fluorophenyl)piperazine (comp. 5c, 150 mg, 87%) as a yellow solid which was used directly in the next step.

### Compound 5d. (1S,2S)-2-(4-(4-chloro-2-fluorophenyl)piperazin-1-yl)cyclohexan-1-amine

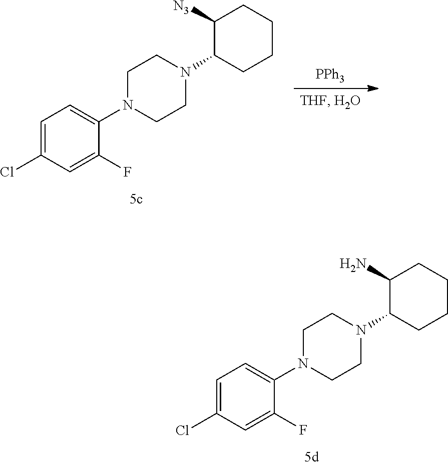

To a solution of 1-((1S,2S)-2-azidocyclohexyl)-4-(4-chloro-2-fluorophenyl)piperazine (150 mg, 0.445 mmol) in THF/H₂O (5 mL/1 mL) was added PPh₃ (140 mg, 0.534 mmol). The mixture was stirred at 50° C. overnight. Then the resulting solution was washed with water (20 mL) and extracted with DCM (20 mL). The combined organic layers were dried over anhydrous Na₂SO₄ and concentrated under reduced pressure. The residue was purified by silica gel column chromatography to give (1S,2S)-2-(4-(4-chloro-2-fluorophenyl)piperazin-1-yl)cyclohexanamine (comp. 5d, 55 mg, 40%) as a white solid, which was used directly in the next step.

### Compound 105. N¹-((1S,2S)-2-(4-(4-chloro-2-fluorophenyl)piperazin-1-yl)cyclohexyl)-N⁴,N⁴-dimethylbenzene-1,4-disulfonamide

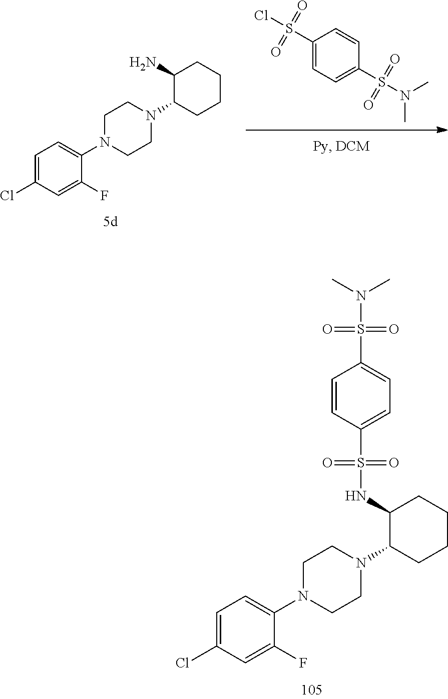 [SMILES: N[C@H]1CCCC[C@@H]1N1CCN(c2ccc(Cl)cc2F)CC1 ; CN(C)S(=O)(=O)c1ccc(S(=O)(=O)Cl)cc1 ; CN(C)S(=O)(=O)c1ccc(S(=O)(=O)N[C@H]2CCCC[C@@H]2N2CCN(c3ccc(Cl)cc3F)CC2)cc1]

To a solution of (1S,2S)-2-(4-(4-chloro-2-fluorophenyl)piperazin-1-yl)cyclohexanamine (55 mg, 0.176 mmol) in DCM (10 mL) was added 4-(N,N-dimethylsulfamoyl)benzenesulfonyl chloride (50 mg, 0.176 mmol) and pyridine (0.1 mL). The mixture was stirred at rt overnight. Then the resulting solution was concentrated and purified by prep-HPLC to give N¹-((1S,2S)-2-(4-(4-chloro-2-fluorophenyl)piperazin-1-yl)cyclohexyl)-N⁴,N⁴-dimethylbenzene-1,4-disulfonamide (comp. 105, 26 mg, 26%) as a white solid. ¹H NMR (400 MHz, CDCl₃) δ 8.08 (d, J=8.4 Hz, 2H), 7.92 (d, J=8.4 Hz, 2H), 7.09-7.00 (m, 2H), 6.83 (t, J=8.8 Hz, 1H), 2.99-2.87 (m, 4H), 2.75 (s, 6H), 2.46 (s, 4H), 2.29 (d, J=11.6 Hz, 2H), 1.82 (dd, J=61.4, 28.3 Hz, 4H), 1.25-1.15 (m, 4H).

### Example 6. 4-(2-(4-(4-chloro-2-fluorophenyl)piperazin-1-yl)benzylsulfonyl)-N,N-dimethylbenzenesulfonamide (Compound 106)

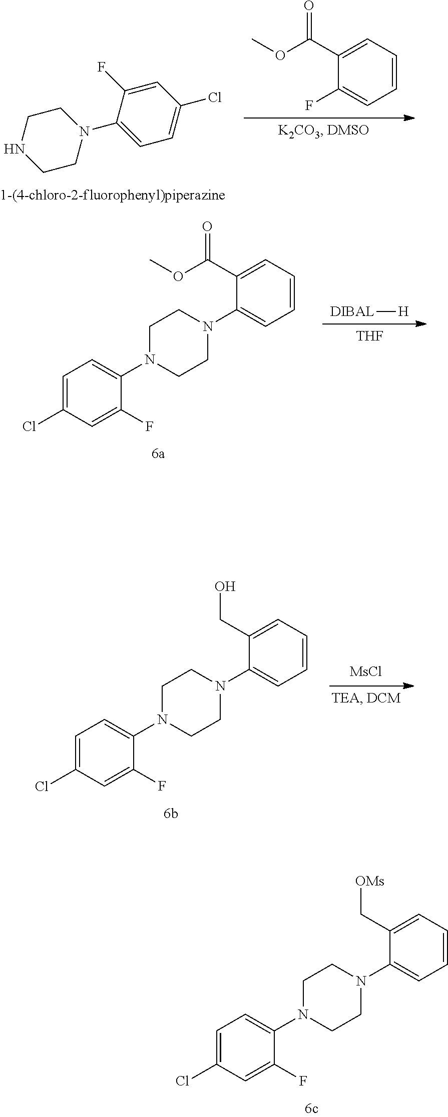

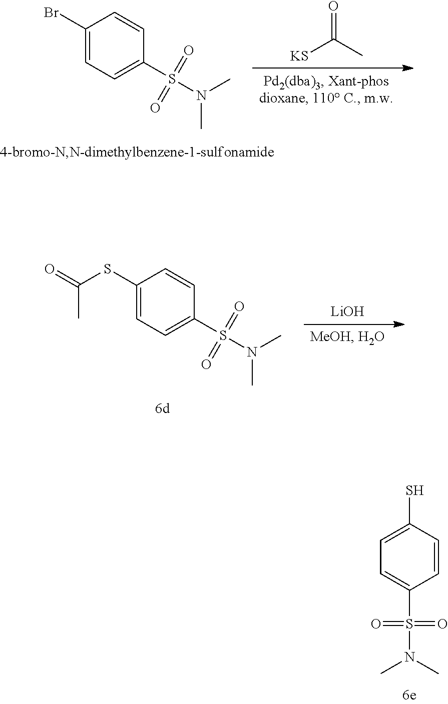

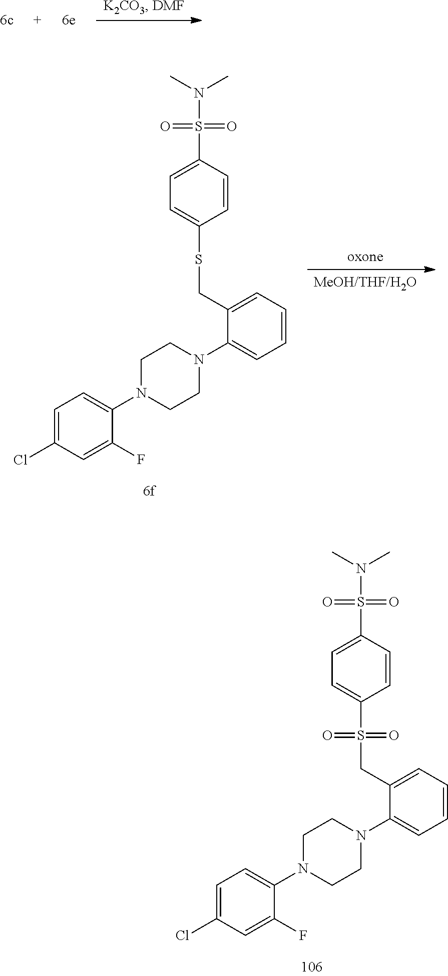

### Compound 6a. Methyl 2-(4-(4-chloro-2-fluorophenyl)piperazin-1-yl)benzoate

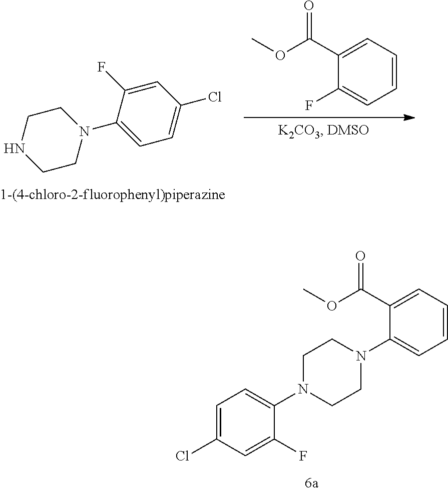

To a solution of 1-(4-chloro-2-fluorophenyl)piperazine (300 mg, 1.4 mmol) in DMSO (8 mL) was added K₂CO₃ (964 mg, 7.0 mmol) and methyl 2-fluorobenzoate (646 mg, 4.2 mmol). After stirring at 160° C. for 2 hrs, the reaction mixture was diluted with EtOAc and washed with saturated aq. NH₄Cl solution and brine, dried over anhydrous Na₂SO₄, filtered and concentrated to dryness. The residue was purified by column chromatography on silica gel (petroleum ether:EtOAc=40:1, v/v) to give methyl 2-(4-(4-chloro-2-fluorophenyl)piperazin-1-yl)benzoate (comp. 6a, 55 mg, 11%) as a yellow solid.

### Compound 6b. (2-(4-(4-chloro-2-fluorophenyl)piperazin-1-yl)phenyl)methanol

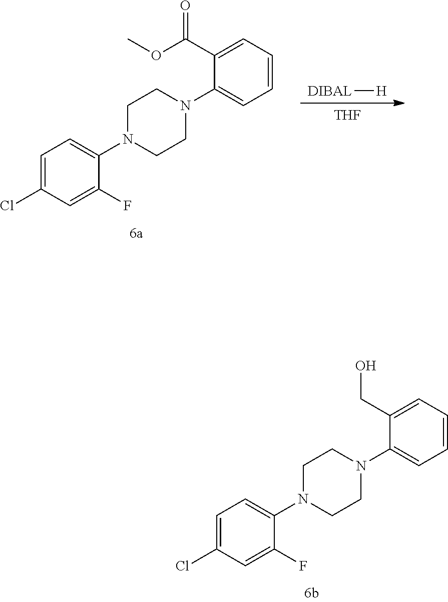

To a solution of methyl 2-(4-(4-chloro-2-fluorophenyl)piperazin-1-yl)benzoate (50 mg, 0.14 mmol) in THF (5 mL) was added dropwise DIBAL-H (0.42 mL, 0.42 mmol, 1 M in toluene) at 0° C. over 3 minutes. After the addition, the resulting solution was stirred at 20° C. for another 3 hrs. After cooling to 0° C., the reaction was treated with EtOAc (20 mL) and 1 N HCl (20 mL). The organic layer was separated and the aqueous layer was extracted with EtOAc (20 mL). The combined organics were concentrated in vacuo and the residue was purified by flash chromatography (silica gel, 0-50% EtOAc in petroleum ether) to give (2-(4-(4-chloro-2-fluorophenyl)piperazin-1-yl)phenyl)methanol (comp. 6b, 40 mg, 87%) as a white solid. LC/MS ESI (m/z): 321 (M+H)⁺.

### Compound 6c. 2-(4-(4-chloro-2-fluorophenyl)piperazin-1-yl)benzyl methanesulfonate

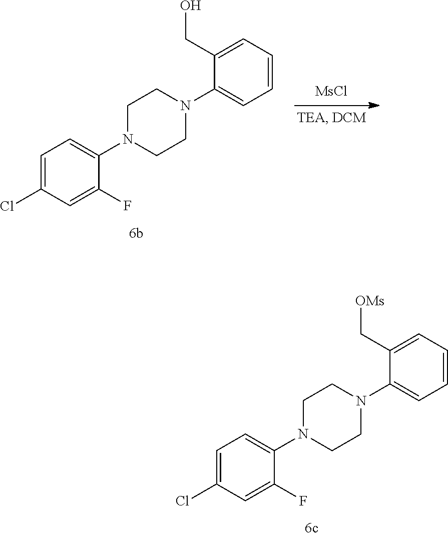 [SMILES: "MsCl" ; OCc1ccccc1N1CCN(c2ccc(Cl)cc2F)CC1 ; "TEA, DCM" ; CS(=O)(=O)OCc1ccccc1N1CCN(c2ccc(Cl)cc2F)CC1]

To a solution of (2-(4-(4-chloro-2-fluorophenyl)piperazin-1-yl)phenyl)methanol (50 mg, 0.16 mmol) in DCM (10 mL) was added TEA (94 mg, 0.94 mmol). After cooling to 0° C., methanesulfonyl chloride (0.024 mL, 0.31 mmol) was added drop wise and the resulting solution was stirred at 0° C. for 0.5 h. Then the mixture was poured into brine. The organic layer was separated and washed with brine. The final organic layer was dried over anhydrous Na₂SO₄ and concentrated in vacuo to afford crude 2-(4-(4-chloro-2-fluorophenyl)piperazin-1-yl)benzyl methanesulfonate (comp. 6c, 62 mg, 99%) which was used directly in the following step. LC/MS ESI (m/z): 399 (M+H)⁺.

### Compound 6d. S-4-(N,N-dimethylsulfamoyl)phenyl ethanethioate

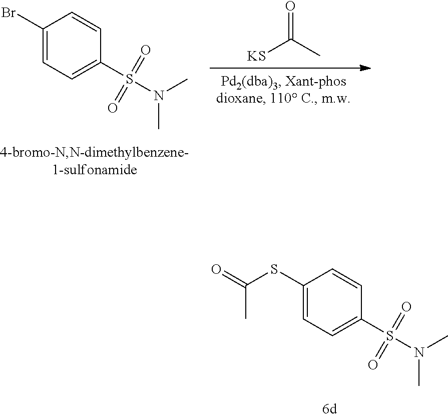

To a solution of 4-bromo-N,N-dimethylbenzene-1-sulfonamide (130 mg, 0.5 mmol) in dioxane (2 mL) were added potassium ethanethioate (66 mg, 0.6 mmol), DIPEA (190 mg, 1.5 mmol), Pd₂(dba)₃ (11 mg, 0.01 mmol) and Xant-Phos (12 mg, 0.02 mmol). Then the mixture was stirred at 160° C. under microwave condition for 50 min. The reaction mixture was diluted with EtOAc and washed with water and brine, dried over anhydrous Na₂SO₄, filtered and concentrated to dryness. The residue was purified by flash chromatography to give S-4-(N,N-dimethylsulfamoyl)phenyl ethanethioate (comp. 6d, 71 mg, 56%) as a yellow solid. LC/MS ESI (m/z): 260 (M+H)⁺.

### Compound 6e. 4-Mercapto-N,N-dimethylbenzenesulfonamide

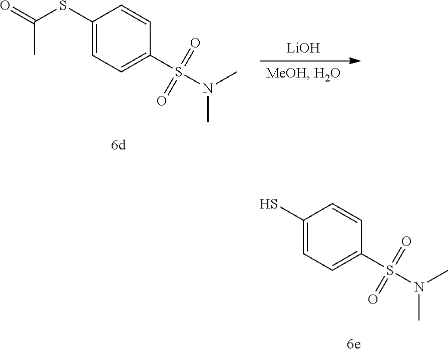

To a solution of S-(4-(N,N-dimethylsulfamoyl)phenyl) ethanethioate (71 mg, 0.3 mmol) in MeOH (3 mL) was added LiOH (6.9 mg, 0.3 mmol) in H₂O (0.5 mL). The mixture was stirred at 25° C. for 0.5 h. Then the reaction mixture was diluted with DCM and washed with water and brine. The final organic was dried over anhydrous Na₂SO₄, filtered and concentrated to dryness. The residue was purified by flash chromatography on silica gel (petroleum ether:EtOAc=30:1) to give 4-mercapto-N,N-dimethylbenzenesulfonamide (comp. 6e, 49 mg, 78%) as a yellow solid. LC/MS ESI (m/z): 218 (M+H)⁺.

### Compound 6f. 4-(2-(4-(4-Chloro-2-fluorophenyl)piperazin-1-yl)benzylthio)-N,N-dimethylbenzenesulfonamide

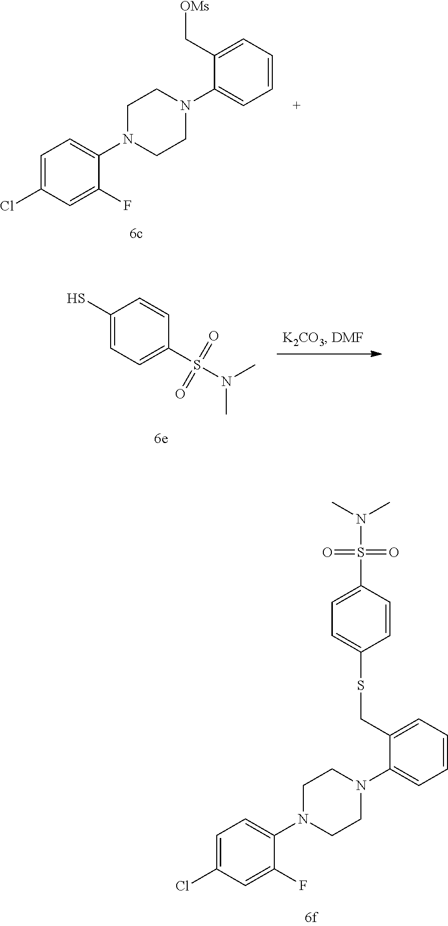

To a solution of crude 2-(4-(4-chloro-2-fluorophenyl)piperazin-1-yl)benzyl methanesulfonate (62 mg, 0.16 mmol) in MeCN (10 mL) were added 4-mercapto-N,N-dimethylbenzenesulfonamide (51 mg, 0.23 mmol) and K₂CO₃ (214 mg, 1.6 mmol). The resulting mixture was stirred at 20° C. overnight. After filtration through celite to remove K₂CO₃, the filtrate was concentrated in vacuo and the residue was purified by prep-TLC to give 4-(2-(4-(4-chloro-2-fluorophenyl)piperazin-1-yl)benzylthio)-N,N-dimethylbenzenesulfonamide (comp. 6f, 60 mg, 74%) as a colorless oil. LC/MS ESI (m/z): 520 (M+H)⁺.

### Compound 106. 4-(2-(4-(4-Chloro-2-fluorophenyl)piperazin-1-yl)benzylsulfonyl)-N,N-dimethylbenzenesulfonamide

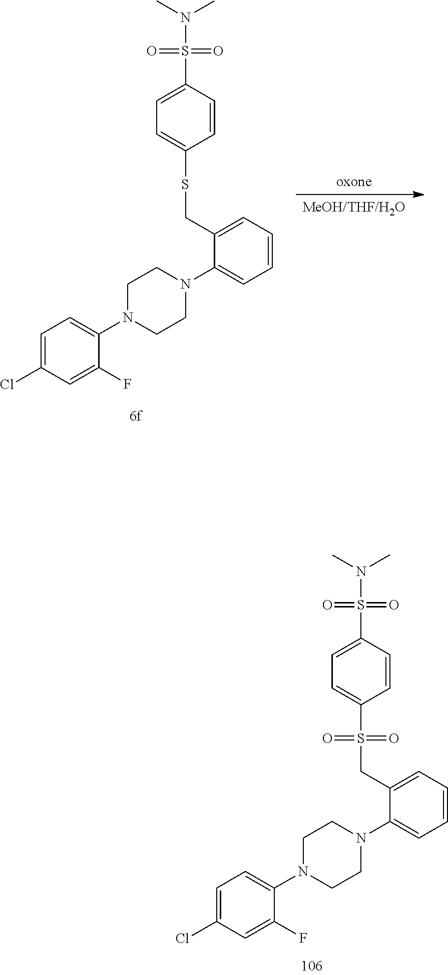 [SMILES: CN(C)S(=O)(=O)c1ccc(SCc2ccccc2N2CCN(c3ccc(Cl)cc3F)CC2)cc1 ; "MeOH/THF/H2O" ; "oxone" ; CN(C)S(=O)(=O)c1ccc(S(=O)(=O)Cc2ccccc2N2CCN(c3ccc(Cl)cc3F)CC2)cc1]

To a solution of 4-(2-(4-(4-chloro-2-fluorophenyl)piperazin-1-yl)benzylthio)-N,N-dimethylbenzenesulfonamide (60 mg, 0.12 mmol) in MeOH (15 mL) was added a solution of Oxone (213 mg, 0.35 mmol) in H₂O (5 mL). The resulting mixture was stirred at 45° C. overnight. The mixture was treated with aq. Na₂S₂O₃ at 25° C. for 1 h before it was poured into DCM. The organic layer was separated, washed with brine and concentrated in vacuo. The residue was purified by prep-HPLC to give 4-(2-(4-(4-chloro-2-fluorophenyl)piperazin-1-yl)benzylsulfonyl)-N,N-dimethylbenzenesulfonamide (comp. 106, 21 mg, 33%) as a white solid. LC/MS ESI (m/z): 552 (M+H)⁺. ¹H NMR (400 MHz, DMSO-d₆) δ 7.90 (d, J=8.5 Hz, 2H), 7.81 (d, J=8.6 Hz, 2H), 7.46 (dd, J=7.7, 1.5 Hz, 1H), 7.38-7.32 (m, 2H), 7.24-7.08 (m, 4H), 4.82 (s, 2H), 3.04-2.94 (m, 4H), 2.63 (s, 6H), 2.61-2.55 (m, 4H).

### Example 7. N¹-(2-((1R,4R)-5-(4-chloro-2-fluorophenyl)-2,5-diazabicyclo[2.2.1]heptan-2-yl)phenyl)-N⁴,N⁴-dimethylbenzene-1,4-disulfonamide (Compound 107)

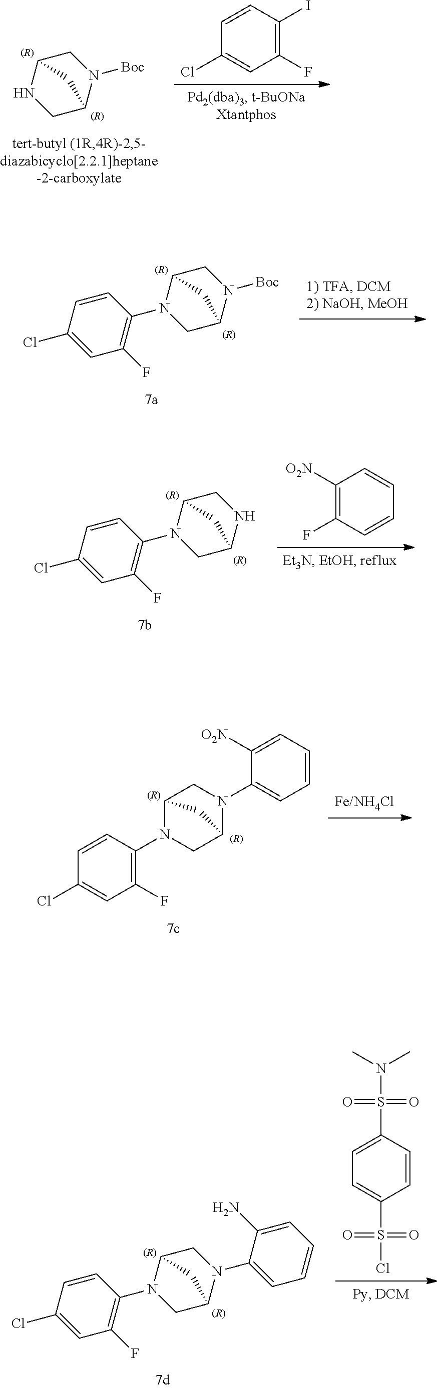

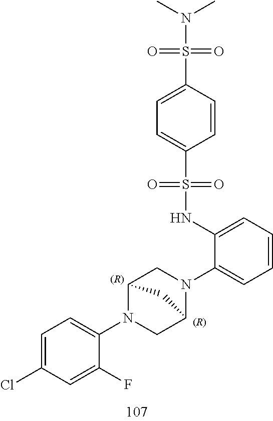

### Compound 7a. tert-butyl (1R,4R)-5-(4-chloro-2-fluorophenyl)-2,5-diazabicyclo[2.2.1]heptane-2-carboxylate

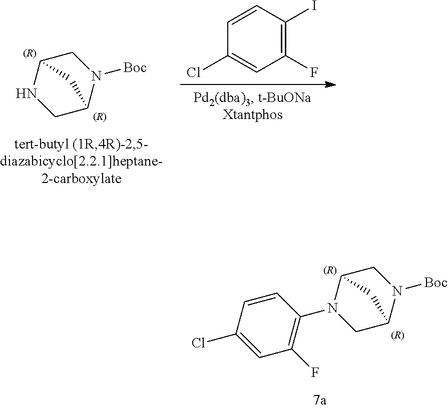

An oven-dried round-bottom flask was purged with N₂ and charged with tert-butyl (1R,4R)-2,5-diazabicyclo[2.2.1]heptane-2-carboxylate (500 mg, 2.52 mmol), Pd₂(dba)₃ (115 mg, 0.12 mmol), Xantphos (73 mg, 0.12 mmol), sodium tert-butoxide (363 mg, 3.78 mmol) and 4-chloro-2-fluoro-1-iodobenzene (647 mg, 2.52 mmol). The mixture was stirred at room temperature for 4 h, then diluted with EtOAc, filtered through celite and concentrated in vacuo. The crude material was purified by flash chromatography (silica gel, 2%˜10% EtOAc in petroleum ether) to afford tert-butyl (1R,4R)-5-(4-chloro-2-fluorophenyl)-2,5-diazabicyclo[2.2.1]heptane-2-carboxylate (comp. 7a, 256 mg, 31%) as a yellow oil. LC/MS ESI (m/z): 327 (M+H)⁺.

### Compound 7b. (1R,4R)-2-(4-chloro-2-fluorophenyl)-2,5-diazabicyclo[2.2.1]heptane

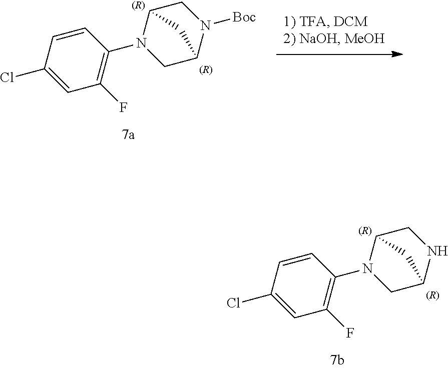

To a solution of tert-butyl (1R,4R)-5-(4-chloro-2-fluorophenyl)-2,5-diazabicyclo[2.2.1]heptane-2-carboxylate (256 mg, 0.78 mmol) in DCM (3 mL) was added TFA (1.5 mL) at rt. The reaction was stirred at rt for 3 h. The mixture was concentrated and diluted with MeOH (10 mL). NaOH (3.5 mL, aq, 2M) was added and the mixture was stirred at rt for 30 min. Then the mixture was concentrated and extracted with DCM (10 mL×3). The organic layer was dried over anhydrous Na₂SO₄ to give crude (1R,4R)-2-(4-chloro-2-fluorophenyl)-2,5-diazabicyclo[2.2.1]heptane (comp. 7b) as a yellow oil, which was used for the next step without further purification.

### Compound 7c. (1R,4R)-2-(4-chloro-2-fluorophenyl)-5-(2-nitrophenyl)-2,5-diazabicyclo[2.2.1]heptane

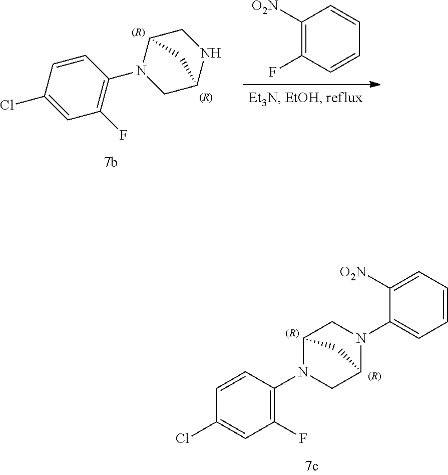

In a sealed tube, a solution of (1R,4R)-2-(4-chloro-2-fluorophenyl)-2,5-diazabicyclo[2.2.1]heptane (˜150 mg, crude), Et₃N (0.15 mL) and 1-fluoro-2-nitrobenzene (89 mg, 0.71 mmol) in EtOH (5 mL) was stirred at 80° C. overnight. The mixture was concentrated under vacuum. The residue was purified by flash chromatography on silica gel (petroleum ether:EtOAc=50:1, v/v) to afford (1R,4R)-2-(4-chloro-2-fluorophenyl)-5-(2-nitrophenyl)-2,5-diazabicyclo[2.2.1]heptane (comp. 7c, 110 mg, 40%) as a yellow oil. LC/MS ESI (m/z): 348 (M+H)⁺.

### Compound 7d. 2-((1R,4R)-5-(4-chloro-2-fluorophenyl)-2,5-diazabicyclo[2.2.1]heptan-2-yl)aniline

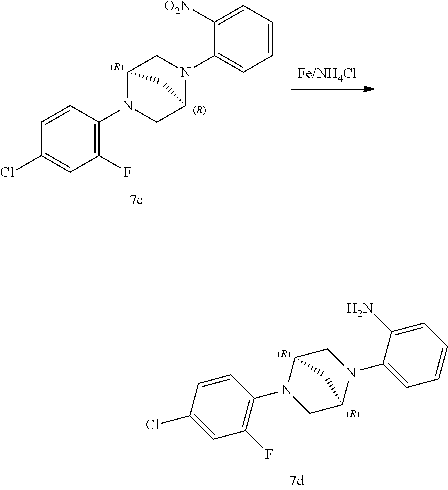

To a solution of (1R,4R)-2-(4-chloro-2-fluorophenyl)-5-(2-nitrophenyl)-2,5-diazabicyclo[2.2.1]heptane (110 mg, 0.31 mmol) in MeOH (5 mL) and saturated aqueous NH₄Cl (2 mL) was added Fe (88 mg, 1.59 mmol). The mixture was heated to reflux and stirred for 3 h. The mixture was cooled to rt and filtered. Solvents were evaporated, and the residue was re-dissolved in 10 mL of DCM. The organic mixture was washed with water twice and the organic layer was dried and concentrated to afford crude 2-[(1R,4R)-5-(4-chloro-2-fluorophenyl)-2,5-diazabicyclo[2.2.1]heptan-2-yl]aniline (comp. 7d, 85 mg) which was used for the next step directly without further purification. LC/MS ESI (m/z): 318 (M+H)⁺.

### Compound 107. N¹-(2-((1R,4R)-5-(4-chloro-2-fluorophenyl)-2,5-diazabicyclo[2.2.1]heptan-2-yl)phenyl)-N⁴,N⁴-dimethylbenzene-1,4-disulfonamide

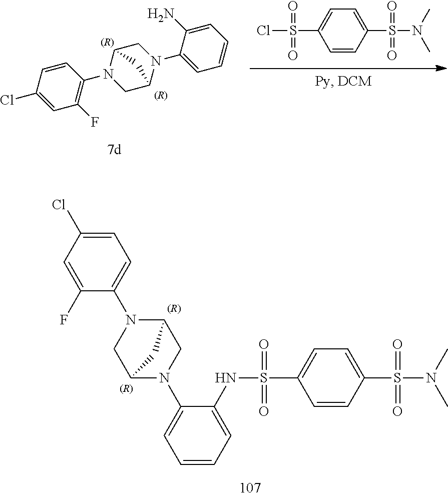

To a solution of 2-[(1R,4R)-5-(4-chloro-2-fluorophenyl)-2,5-diazabicyclo[2.2.1]heptan-2-yl]aniline (90 mg, 0.28 mmol) in DCM (5 mL) and pyridine (0.02 mL, 0.26 mmol) was added 4-(N,N-dimethylsulfamoyl)benzenesulfonyl chloride (91 mg, 0.32 mmol) at 0° C. The mixture was stirred at rt overnight. The mixture was then filtered and the filtrate was concentrated. The residue was purified by flash chromatography on silica gel (petroleum ether:EtOAc=50:1 to 2:3, v/v) to afford N¹-(2-((1R,4R)-5-(4-chloro-2-fluorophenyl)-2,5-diazabicyclo[2.2.1]heptan-2-yl)phenyl)-N⁴,N⁴-dimethylbenzene-1,4-disulfonamide (comp. 107.44 mg, 29%) as a white solid. LC/MS ESI (m/z): 565 (M+H)⁺. ¹H NMR (400 MHz, DMSO-d₆) δ 9.51 (s, 1H), 7.99-7.75 (m, 4H), 7.19 (dd, J=13.5, 2.5 Hz, 1H), 7.07-7.01 (m, 2H), 6.77-6.69 (m, 2H), 6.50 (td, J=7.5, 1.3 Hz, 1H), 6.43 (dd, J=7.8, 1.7 Hz, 1H), 4.46 (d, J=19.5 Hz, 2H), 3.75 (dd, J=9.8, 2.1 Hz, 1H), 3.71-3.64 (m, 1H), 3.23 (dt, J=9.5, 2.4 Hz, 1H), 3.18 (d, J=9.8 Hz, 1H), 2.64 (s, 6H), 1.94 (d, J=9.4 Hz, 1H), 1.76 (d, J=9.3 Hz, 1H).

### Example 8. N¹-(2-(1-(4-chloro-2-fluorophenyl)piperidin-4-yl)phenyl)-N⁴,N⁴-dimethylbenzene-1,4-disulfonamide (Compound 108)

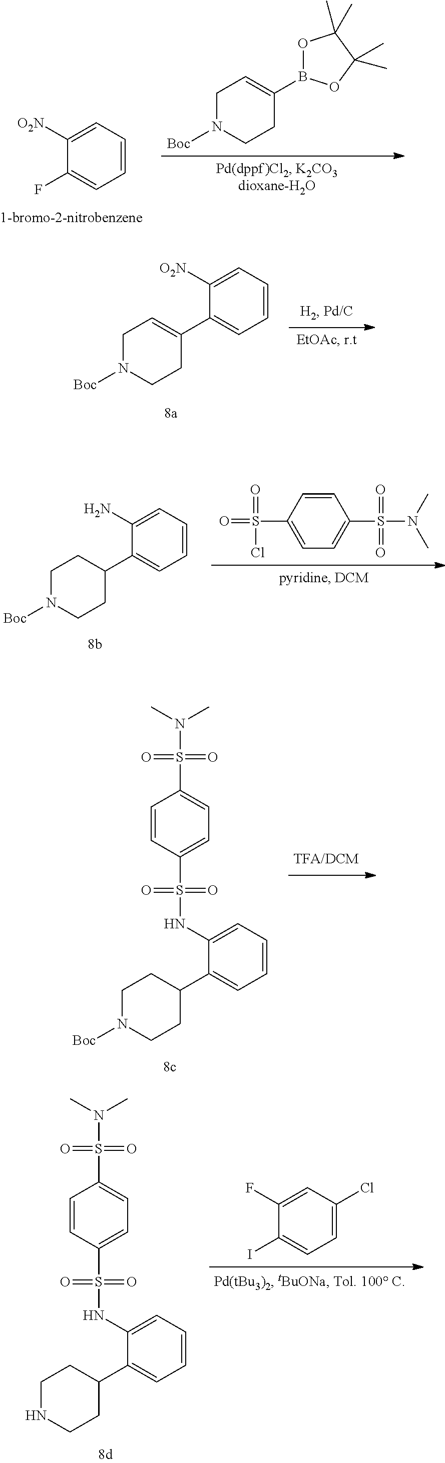

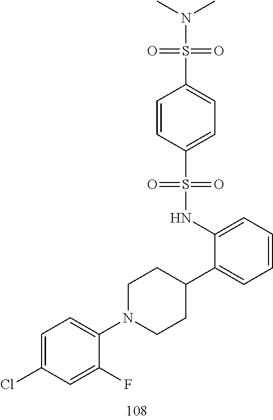 [SMILES: CN(C)S(=O)(=O)c1ccc(S(=O)(=O)Nc2ccccc2C2CCN(c3ccc(Cl)cc3F)CC2)cc1]

### Compound 8a. tert-Butyl 4-(2-nitrophenyl)-3,6-dihydropyridine-1(2H)-carboxylate

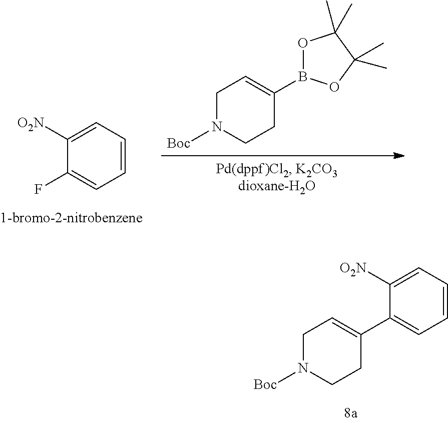 [SMILES: O=[N+]([O-])c1ccccc1F ; CC(C)(C)OC(=O)N1CC=C(B2OC(C)(C)C(C)(C)O2)CC1 ; "Pd(dppf)Cl2, K2CO3dioxane-H2O" ; CC(C)(C)OC(=O)N1CC=C(c2ccccc2[N+](=O)[O-])CC1]

To a solution of 1-bromo-2-nitrobenzene (700 mg, 3.48 mmol) in dioxane (15 mL) and H₂O (3 mL) was added tert-butyl 4-(4,4,5,5-tetramethyl-1,3,2-dioxaborolan-2-yl)-3,6-dihydropyridine-1(2H)-carboxylate (1.18 g, 3.83 mmol), K₂CO₃ (1.44 g, 10.44 mmol) and Pd(dppf)Cl₂ (127 mg, 0.17 mmol). The mixture was purged with N₂ for three times and stirred at 100° C. for 16 h. The mixture was concentrated and purified by flash chromatography to give tert-butyl 4-(2-nitrophenyl)-3,6-dihydropyridine-1(2H)-carboxylate (comp. 8a, 1.0 g, 94%) as a clear oil. LC/MS ESI (m/z): 305 (M+H)⁺.

### Compound 8b. tert-butyl 4-(2-aminophenyl)piperidine-1-carboxylate

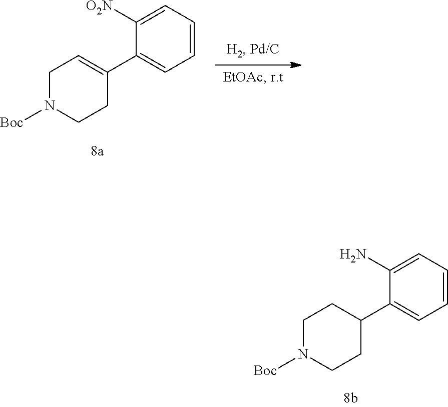 [SMILES: CC(C)(C)OC(=O)N1CC=C(c2ccccc2[N+](=O)[O-])CC1 ; "EtOAc, r.t" ; "H2, Pd/C" ; CC(C)(C)OC(=O)N1CCC(c2ccccc2N)CC1]

To a solution of tert-butyl 4-(2-nitrophenyl)-3,6-dihydropyridine-1(2H)-carboxylate (1.0 g, 3.29 mmol) in EtOAc (20 mL) was added Pd/C (500 mg). The mixture was stirred at rt under 50 Psi of H₂ for 16 h. The mixture was filtered through celite and the filter cake was washed twice with EtOAc. The combined organic layers were concentrated in vacuo to give crude tert-butyl 4-(2-aminophenyl)piperidine-1-carboxylate (comp. 8b, 850 mg, 94%) as a solid, which was used directly in the next step. LC/MS ESI (m/z): 277 (M+H)⁺.

### Compound 8c. tert-butyl-4-(2-((4-(N,N-dimethylsulfamoyl)phenyl)sulfonamido)phenyl)piperidine-1-carboxylate

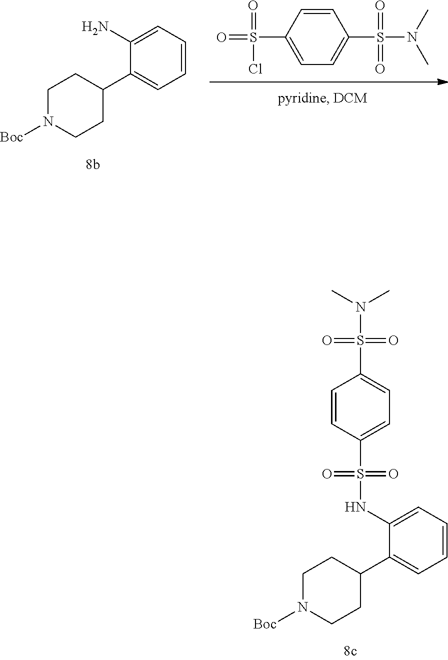

To a solution of tert-butyl 4-(2-aminophenyl)piperidine-1-carboxylate (850 mg, 3.08 mmol) in DCM (15 mL) was added 4-(N,N-dimethylsulfamoyl)benzenesulfonyl chloride (959 mg, 3.39 mmol) and pyridine (486 mg, 6.16 mmol) at 0° C. Then the mixture was stirred at rt for 2 h. The mixture was concentrated and purified by flash chromatography to give tert-butyl-4-(2-((4-(N,N-dimethylsulfamoyl)phenyl)sulfonamido)phenyl)piperidine-1-carboxylate (comp. 8c, 1.2 g, 74%) as an off-white solid. LC/MS ESI (m/z): 524 (M+H)⁺.

### Compound 8d. N¹,N¹-dimethyl-N⁴-(2-(piperidin-4-yl)phenyl)benzene-1,4-disulfonamide

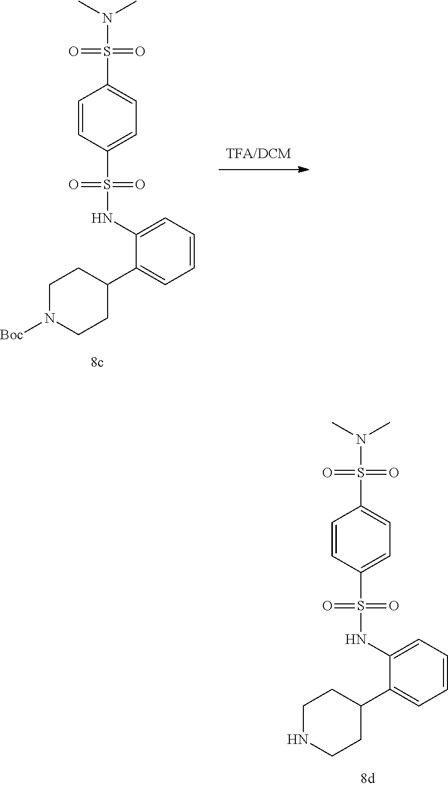 [SMILES: CN(C)S(=O)(=O)c1ccc(S(=O)(=O)Nc2ccccc2C2CCN(C(=O)OC(C)(C)C)CC2)cc1 ; CN(C)S(=O)(=O)c1ccc(S(=O)(=O)Nc2ccccc2C2CCNCC2)cc1]

To a solution of 4-(2-((4-(N,N-dimethylsulfamoyl)phenyl)sulfonamido)phenyl)piperidine-1-carboxylate (850 mg, 1.62 mmol) in DCM (10 mL) were added TFA (3 mL). The reaction mixture was stirred at rt for 1 h. The mixture was concentrated, diluted with DCM (50 mL) and washed with sat. NaHCO₃. The separated organic layer was dried over anhydrous Na₂SO₄, filtered and concentrated to give crude N¹,N¹-dimethyl-N⁴-(2-(piperidin-4-yl)phenyl)benzene-1,4-disulfonamide (comp. 8d, 600 mg, 87%) as an off-white solid. LC/MS ESI (m/z): 424 (M+H)⁺.

### Compound 108. N¹-(2-(1-(4-chloro-2-fluorophenyl)piperidin-4-yl)phenyl)-N⁴,N⁴-dimethylbenzene-1,4-disulfonamide

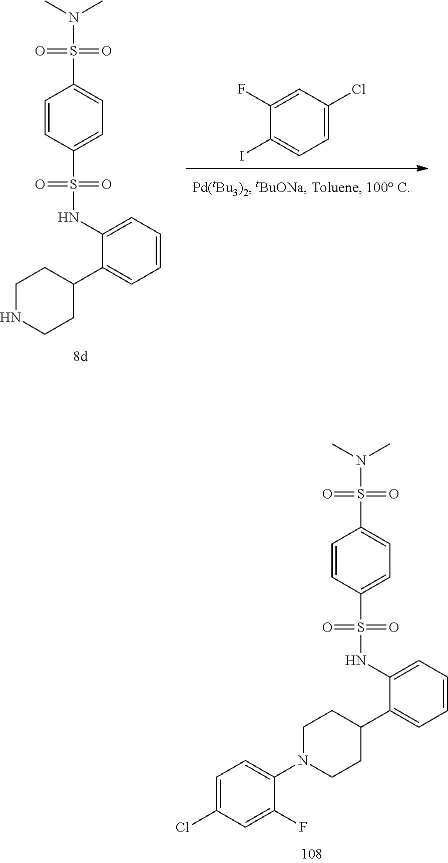 [SMILES: CN(C)S(=O)(=O)c1ccc(S(=O)(=O)Nc2ccccc2C2CCNCC2)cc1 ; Fc1cc(Cl)ccc1I ; CN(C)S(=O)(=O)c1ccc(S(=O)(=O)Nc2ccccc2C2CCN(c3ccc(Cl)cc3F)CC2)cc1]

To a solution of N¹,N¹-dimethyl-N⁴-(2-(piperidin-4-yl)phenyl)benzene-1,4-disulfonamide (500 mg, 1.18 mmol) in toluene (10 mL) was added 4-chloro-2-fluoro-1-iodobenzene (1.2 g, 4.72 mmol), Pd(^{t}Bu₃)₂ (90 mg, 0.18 mmol) and ^{t}BuONa (340 mg, 3.54 mmol). The mixture was purged with N₂ for three times and stirred at 100° C. for 16 h. The resulting solution was concentrated, diluted with water, and extracted with ethyl acetate twice. The combined organic layers were dried over anhydrous Na₂SO₄ and concentrated. The residue was purified by prep-HPLC to give N¹-(2-(1-(4-chloro-2-fluorophenyl)piperidin-4-yl)phenyl)-N⁴,N⁴-dimethylbenzene-1,4-disulfonamide (comp. 108, 10 mg, 1.5%) as an off-white solid. LC/MS ESI (m/z): 552 (M+H)⁺. ¹H NMR (400 MHz, DMSO-d₆) δ 10.09 (s, 1H), 7.95-7.97 (m, 2H), 7.87-7.89 (m, 2H), 7.30-7.33 (m, 2H), 7.29-7.30 (m, 1H), 7.14-7.25 (m, 2H), 7.03-7.07 (m, 2H), 3.30-3.32 (m, 2H), 3.26-3.29 (m, 1H), 2.70 (s, 6H), 2.51-2.55 (m, 2H) 1.58-1.62 (m, 2H), 1.19-1.22 (m, 2H).

### Example 9. N¹-((1S,2R)-2-(4-(4-chloro-2-fluorophenyl)piperazin-1-yl)cyclohexyl)-N⁴,N⁴-dimethylbenzene-1,4-disulfonamide (Compound 109)

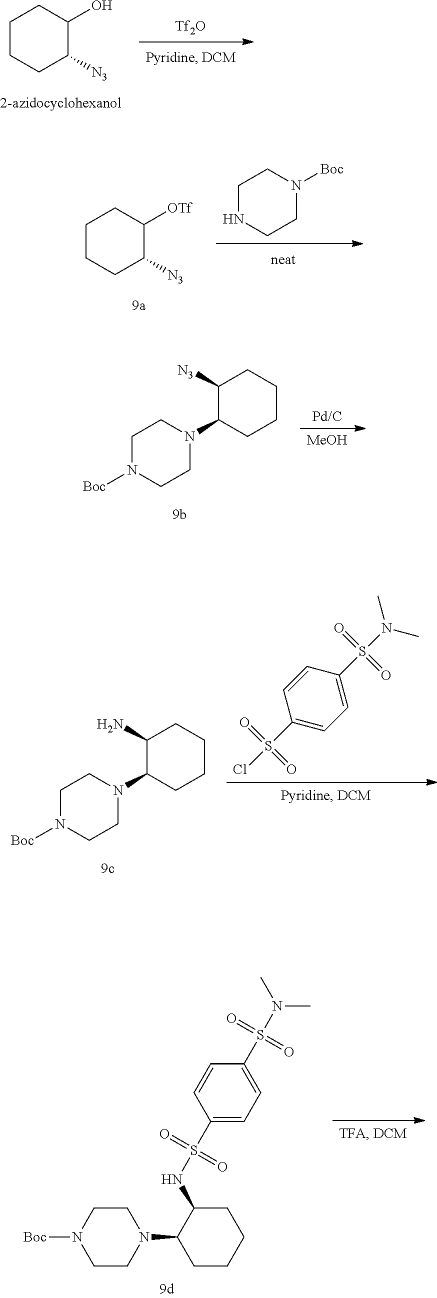

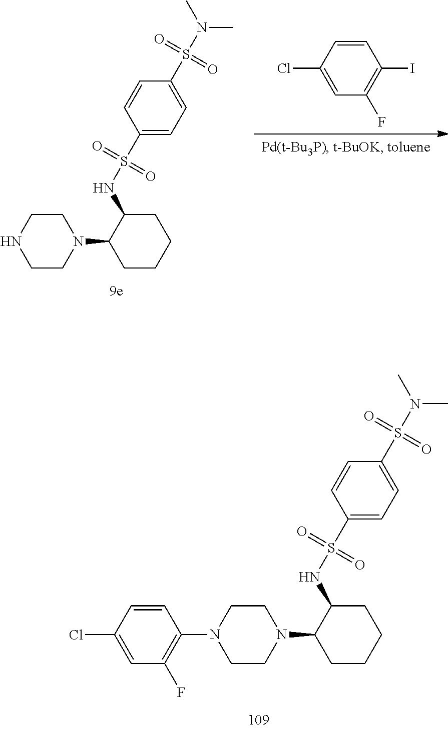

### Compound 9a. (2R)-2-azidocyclohexyl trifluoromethanesulfonate

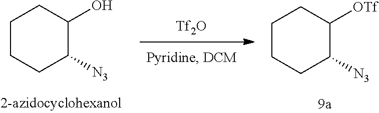 [SMILES: "Tf2O" ; [N-]=[N+]=N[C@@H]1CCCCC1O ; [N-]=[N+]=N[C@@H]1CCCCC1OS(=O)(=O)C(F)(F)F]

A mixture of 2-azidocyclohexanol (5.5 g, 39.0 mmol) and pyridine (4.62 g, 58.5 mmol) in 150 mL DCM at 0° C. was treated with Tf₂O (13.19 g, 46.8 mmol) with stirring. After 1 hr, the ice bath was removed and stirring was continued for additional 1 h. Then the solution was poured into water and extracted with DCM and the organic layer was washed several times with water. After drying, the organic layer was evaporated and the residue (comp. 9a, 5.5 g, crude) was used in the next step directly.

### Compound 9b. tert-Butyl 4-((1R,2S)-2-azidocyclohexyl)piperazine-1-carboxylate

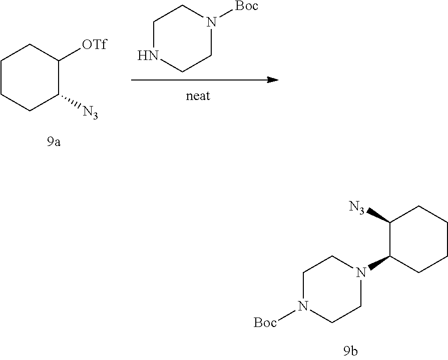 [SMILES: [N-]=[N+]=N[C@@H]1CCCCC1OS(=O)(=O)C(F)(F)F ; CC(C)(C)OC(=O)N1CCNCC1 ; CC(C)(C)OC(=O)N1CCN([C@@H]2CCCC[C@@H]2N=[N+]=[N-])CC1]

A mixture of (2R)-2-azidocyclohexyl trifluoromethanesulfonate (5.5 g, 20 mmol) and tert-butyl piperazine-1-carboxylate (11.2 g, 60.3 mmol) was heated at 120° C. for 5 h. The mixture was purified by flash column chromatography to give tert-butyl 4-((1R,2S)-2-azidocyclohexyl)piperazine-1-carboxylate (comp. 9b, 1.2 g, 19%).

### Compound 9c. tert-Butyl 4-((1R,2S)-2-aminocyclohexyl)piperazine-1-carboxylate

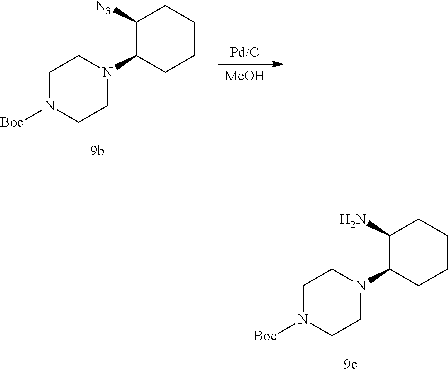 [SMILES: "MeOH" ; CC(C)(C)OC(=O)N1CCN([C@@H]2CCCC[C@@H]2N=[N+]=[N-])CC1 ; "Pd/C" ; CC(C)(C)OC(=O)N1CCN([C@@H]2CCCC[C@@H]2N)CC1]

To a solution of tert-butyl 4-((1R,2S)-2-azidocyclohexyl)piperazine-1-carboxylate (1.2 g, 3.88 mmol) in methanol (20 mL) was added 10% Pd/C (100 mg). Then the mixture was stirred under H₂ overnight. The reaction mixture was filtered through a celite pad and the filtrate was concentrated and purified by flash column chromatography to give tert-butyl 4-((1R,2S)-2-aminocyclohexyl)piperazine-1-carboxylate (comp. 9c, 1.0 g, 90%).

### Compound 9d. tert-Butyl-4-((1R,2S)-2-(4-(N,N-dimethylsulfamoyl)phenylsulfonamido)cyclohexyl)piperazine-1-carboxylate

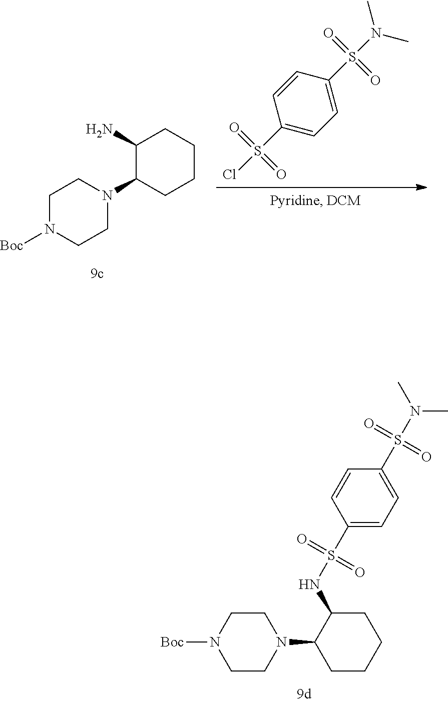

To a solution of tert-butyl 4-((1R,2S)-2-aminocyclohexyl)piperazine-1-carboxylate (1.0 g, 3.5 mmol) in DCM (20 mL) was added pyridine (418 mg, 5.29 mmol), followed by 4-(N,N-dimethylsulfamoyl)benzene-1-sulfonyl chloride (1.20 g, 4.23 mmol) at 0° C. The mixture was stirred at 0° C. for 1 h. The reaction mixture was then diluted with DCM, washed with water twice, dried over anhydrous Na₂SO₄ and concentrated. The residue was purified by flash column chromatography to give tert-butyl 4-((1R,2S)-2-(4-(N,N-dimethylsulfamoyl)phenylsulfonamido)cyclohexyl)piperazine-1-carboxylate (comp. 9d, 700 mg, 37%).

### Compound 9e. N¹,N¹-Dimethyl-N⁴-((1S,2R)-2-(piperazin-1-yl)cyclohexyl)benzene-1,4-disulfonamide

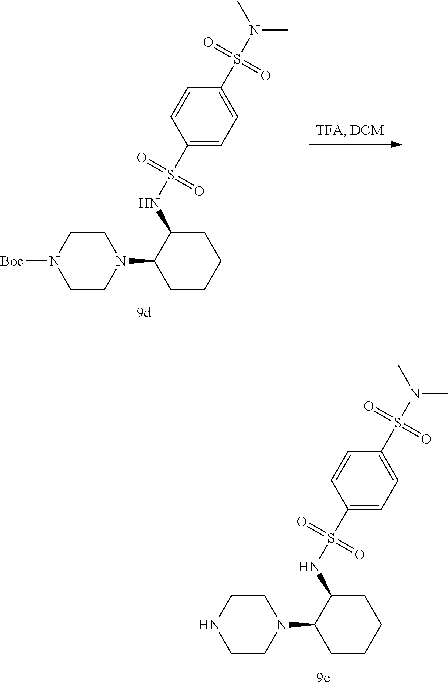

To a solution of tert-butyl 4-((1R,2S)-2-(4-(N,N-dimethylsulfamoyl)phenylsulfonamido)cyclohexyl)piperazine-1-carboxylate (700 mg, 1.32 mmol) in DCM (10 mL) was added TFA (3 mL). Then the mixture was stirred at rt for 2 h. The solvents were evaporated, re-dissolved in DCM and washed with saturated NaHCO₃ solution and brine. Then the organic phase was concentrated to give N¹,N¹-dimethyl-N⁴-((1S,2R)-2-(piperazin-1-yl)cyclohexyl)benzene-1,4-disulfonamide (comp. 9e, 400 mg, 70%).

### Compound 109. N¹-((1S,2R)-2-(4-(4-Chloro-2-fluorophenyl)piperazin-1-yl)cyclohexyl)-N⁴,N⁴-dimethylbenzene-1,4-disulfonamide

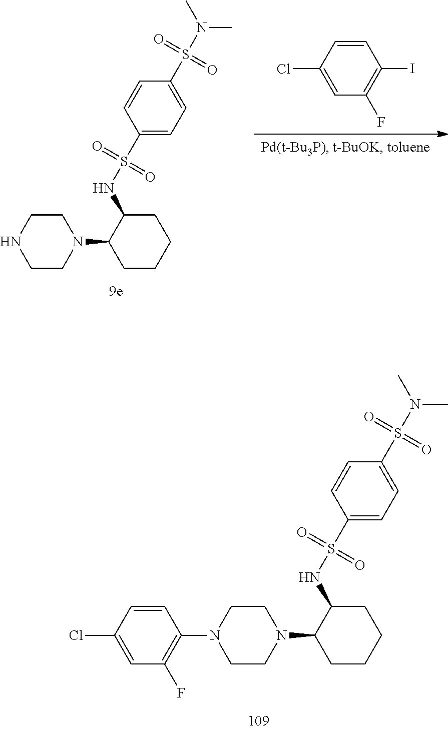

To a mixture of 4-chloro-2-fluoro-1-iodobenzene (416 mg, 1.62 mmol) and N¹,N¹-dimethyl-N⁴-((1S,2R)-2-(piperazin-1-yl)cyclohexyl)benzene-1,4-disulfonamide (350 mg, 0.81 mmol) in toluene (10 mL) was added t-BuOK (3 mL, 1 N in THF) and Pd(t-Bu₃P)₂ (40 mg, 0.08 mmol). Then the mixture was stirred at 100° C. under N₂ overnight. The mixture was diluted with EtOAc, washed with water and brine. Then the organic layer was concentrated and purified by flash column chromatography to give N¹-((1S,2R)-2-(4-(4-chloro-2-fluorophenyl)piperazin-1-yl)cyclohexyl)-N⁴,N⁴-dimethylbenzene-1,4-disulfonamide (comp. 109, 25 mg, 5.5%). ¹H NMR (400 MHz, CDCl₃) δ 8.09 (d, J=8.5 Hz, 2H), 7.92 (d, J=8.5 Hz, 2H), 7.10-6.99 (m, 2H), 6.84 (t, J=8.4 Hz, 1H), 3.66 (s, 1H), 2.97 (s, 4H), 2.80-2.51 (m, 11H), 2.29 (d, J=11.6 Hz, 1H), 2.13 (d, J=14.4 Hz, 1H), 1.88-1.78 (m, 2H), 1.48-1.16 (m, 4H).

### Example 10. (S)-N¹-(2-((1-(4-Chloro-2-fluorophenyl)pyrrolidin-3-yl)(methyl)amino)phenyl)-N⁴,N⁴-dimethylbenzene-1,4-disulfonamide (Compound 110)

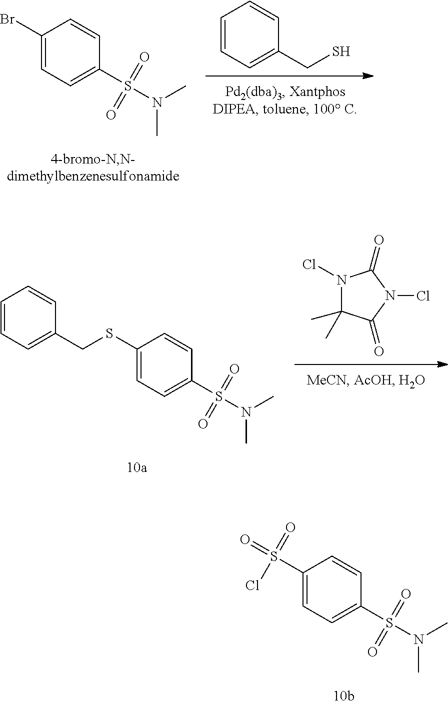 [SMILES: CN(C)S(=O)(=O)c1ccc(Br)cc1 ; SCc1ccccc1 ; CN(C)S(=O)(=O)c1ccc(SCc2ccccc2)cc1 ; CC1(C)C(=O)N(Cl)C(=O)N1Cl ; CN(C)S(=O)(=O)c1ccc(S(=O)(=O)Cl)cc1]

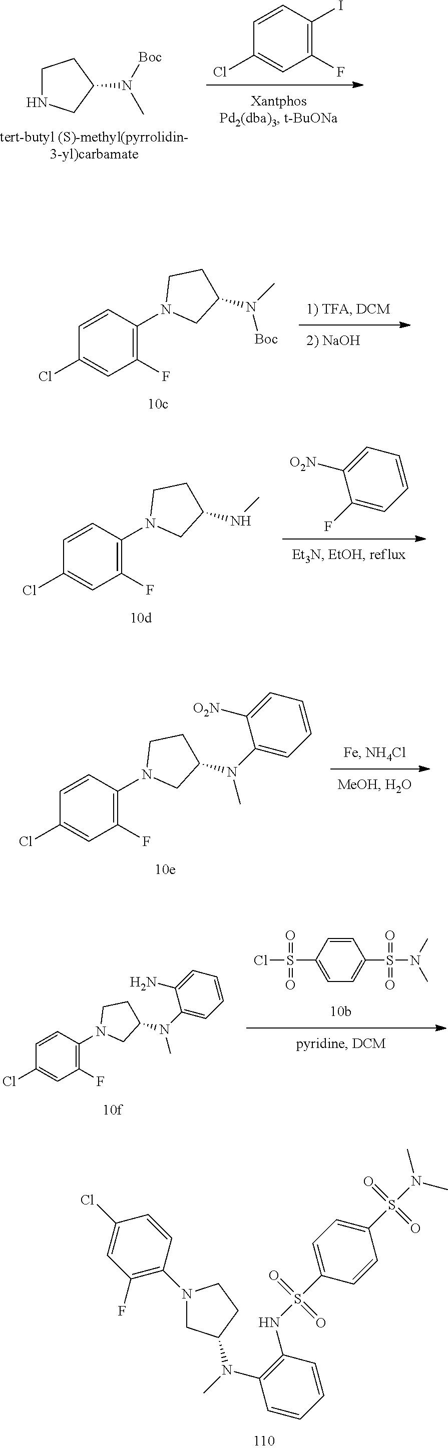

### Compound 10a. 4-(Benzylthio)-N,N-dimethylbenzenesulfonamide

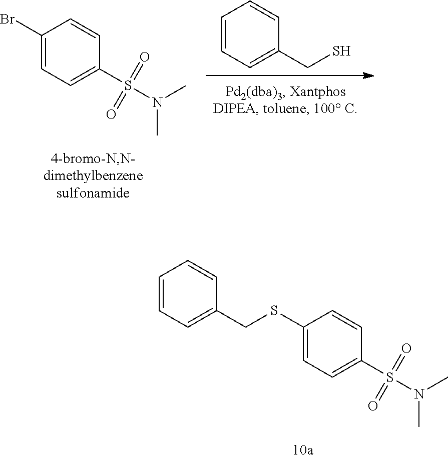 [SMILES: CN(C)S(=O)(=O)c1ccc(Br)cc1 ; SCc1ccccc1 ; CN(C)S(=O)(=O)c1ccc(SCc2ccccc2)cc1]

To a solution of 4-bromo-N,N-dimethylbenzenesulfonamide (2 g, 7.57 mmol) in toluene (80 mL) was added phenylmethanethiol (1.4 g, 11.35 mmol), Pd₂(dba)₃ (346 mg, 0.38 mmol), Xantphos (440 mg, 0.76 mmol) and DIPEA (6.6 mL, 37.85 mmol). The resulting mixture was heated to 100° C. overnight. After being cooled to room temperature, solvent was removed and the residue was purified by flash chromatography (silica gel, 0˜30% ethyl acetate in petroleum ether) to afford 4-(benzylthio)-N,N-dimethylbenzenesulfonamide (comp. 10a, 2.1 g, 90%) as a solid. LC/MS ESI (m/z): 308 (M+H)⁺.

### Compound 10b. 4-(N,N-Dimethylsulfamoyl)benzenesulfonyl chloride

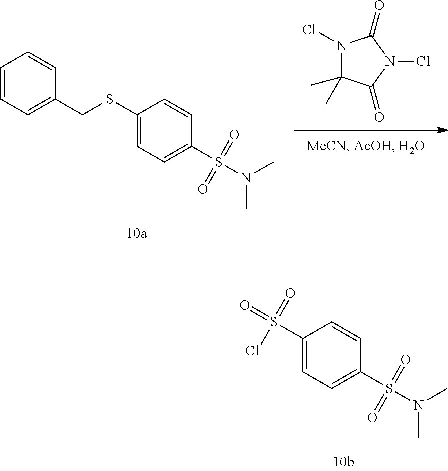 [SMILES: CN(C)S(=O)(=O)c1ccc(SCc2ccccc2)cc1 ; CC1(C)C(=O)N(Cl)C(=O)N1Cl ; CN(C)S(=O)(=O)c1ccc(S(=O)(=O)Cl)cc1]

At 0° C., to a solution of 4-(benzylthio)-N,N-dimethylbenzenesulfonamide (2.1 g, 6.84 mmol) in MeCN (64 mL), was added AcOH (2.4 mL) and water (1.6 mL) was added 1,3-dichloro-5,5-dimethylimidazolidine-2,4-dione (2.7 g, 13.67 mmol) in portions. After being stirred at the same temperature for 20 minutes, the reaction was portioned between DCM and water, organic layer was separated, aqueous layer was extracted with DCM twice, the combined organic layers were washed with brine, dried over Na2SO4, filtered and concentrated. The residue was purified by flash chromatography (silica gel, 0˜25% ethyl acetate in petroleum ether) to afford 4-(N,N-dimethylsulfamoyl)benzenesulfonyl chloride (comp. 10b, 1.9 g, 98%) as a white solid.

### Compound 10c. tert-Butyl (S)-(1-(4-chloro-2-fluorophenyl)pyrrolidin-3-yl)(methyl)carbamate

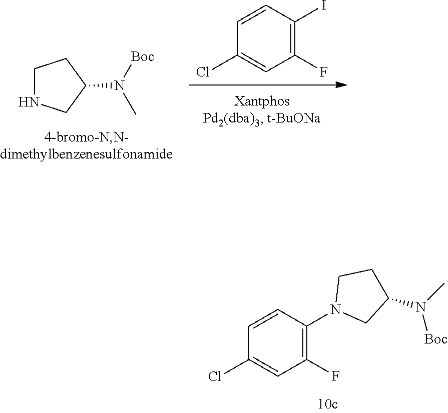 [SMILES: CN(C(=O)OC(C)(C)C)[C@H]1CCNC1 ; Fc1cc(Cl)ccc1I ; CN(C(=O)OC(C)(C)C)[C@H]1CCN(c2ccc(Cl)cc2F)C1]

To a solution of tert-butyl (S)-methyl(pyrrolidin-3-yl)carbamate (500 mg, 2.5 mmol) in toluene (20 mL) was added 4-chloro-2-fluoro-1-iodobenzene (641 mg, 2.5 mmol), Pd2(dba)3 (229 mg, 0.25 mmol), Xantphos (216 mg, 0.38) and sodium tert-butoxide (481 mg, 5 mmol), the resulting mixture was heated to 110° C. overnight. After being cooled down to room temperature, solvent was removed and the residue was purified by flash chromatography (silica gel, 0-50% ethyl acetate in petroleum ether) to afford tert-butyl (S)-(1-(4-chloro-2-fluorophenyl)pyrrolidin-3-yl)(methyl)carbamate (comp. 10c, 160 mg, 19%) as an oil. LC/MS ESI m/z: 329 (M+H)⁺.

### Compound 10d. (S)-1-(4-chloro-2-fluorophenyl)-N-methylpyrrolidin-3-amine

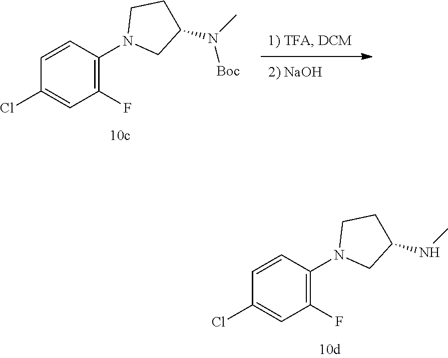 [SMILES: CN(C(=O)OC(C)(C)C)[C@H]1CCN(c2ccc(Cl)cc2F)C1 ; CN[C@H]1CCN(c2ccc(Cl)cc2F)C1]

At 0° C., to a solution of tert-butyl (S)-(1-(4-chloro-2-fluorophenyl)pyrrolidin-3-yl)(methyl)carbamate (160 mg, 0.49 mmol) in DCM (5 mL) was added TFA (1 mL), the resulting mixture was stirred at room temperature for 2 h. After removal of solvent, the residue was diluted with DCM, washed with 1 M NaOH to PH 8, the organic layer was extracted with DCM twice, the combined organic layers were washed with brine, dried over Na2SO4, filtered and concentrated. The residue was used to the next step directly. Comp. 10d, LC/MS ESI m/z: 229 (M+H)⁺.

### Compound 10e. (S)-1-(4-chloro-2-fluorophenyl)-N-methylpyrrolidin-3-amine

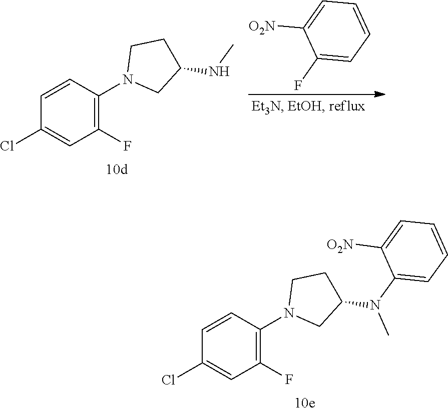

To a solution of (S)-1-(4-chloro-2-fluorophenyl)-N-methylpyrrolidin-3-amine (111 mg, 0.49 mmol) in EtOH (10 mL) was added 1-fluoro-2-nitrobenzene (83 mg, 0.59 mmol) and Et₃N (0.14 mL, 0.98 mmol), the resulting mixture was heated to reflux overnight. After being cooled down to room temperature, solvent was removed, the residue was purified by flash chromatography (silica gel, 0˜30% ethyl acetate in petroleum ether) to afford (S)-1-(4-chloro-2-fluorophenyl)-N-methyl-N-(2-nitrophenyl)pyrrolidin-3-amine (comp. 10e, 140 mg, 82%) as an oil. LC/MS ESI m/z: 350 (M+H)⁺.

### Compound 10f. (S)-N¹-(1-(4-chloro-2-fluorophenyl)pyrrolidin-3-yl)-N¹-methylbenzene-1,2-diamine

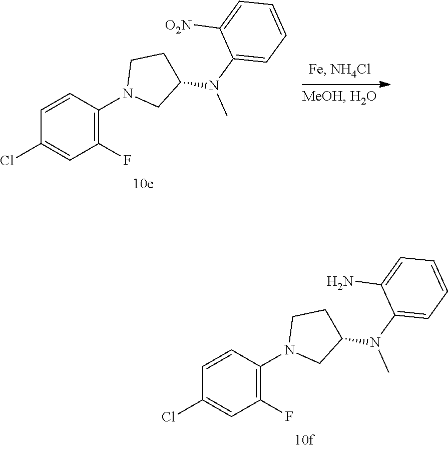 [SMILES: CN(c1ccccc1[N+](=O)[O-])[C@H]1CCN(c2ccc(Cl)cc2F)C1 ; CN(c1ccccc1N)[C@H]1CCN(c2ccc(Cl)cc2F)C1]

The mixture of (S)-1-(4-chloro-2-fluorophenyl)-N-methyl-N-(2-nitrophenyl)pyrrolidin-3-amine (140 mg, 0.40 mmol), Fe (224 mg, 4 mmol), NH₄Cl (321 mg, 6 mmol) in MeOH (10 mL) and water (2 mL) was heated to 50° C. overnight. After being cooled down to room temperature and filtered, the filtrate was concentrated, residue was diluted with DCM, washed with NaHCO₃(aq.), dried over Na₂SO₄, filtered and concentrated, the residue was purified by flash chromatography (silica gel, 0˜100% ethyl acetate in petroleum ether) to afford (S)-N¹-(1-(4-chloro-2-fluorophenyl)pyrrolidin-3-yl)-N¹-methylbenzene-1,2-diamine (comp. 10f, 110 mg, 86%) as an oil. LC/MS ESI m/z: 320 (M+H)⁺.

### Compound 110. (S)-N¹-(2-((1-(4-chloro-2-fluorophenyl)pyrrolidin-3-yl)(methyl)amino)phenyl)-N⁴,N⁴-dimethylbenzene-1,4-disulfonamide

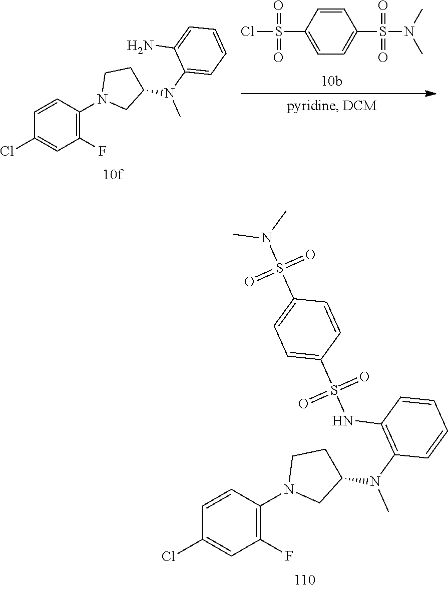

At 0° C., to a solution of 10f (110 mg, 0.34 mmol) in DCM (8 mL) was added pyridine (0.06 mL, 0.7 mmol), followed by 10b (145 mg, 0.51 mmol), the resulting mixture was stirred at room temperature overnight. The reaction was quenched with water, extracted with DCM twice, the combined organic layers were washed with brine, dried over Na₂SO₄, filtered and concentrated. The residue was purified by flash chromatography (silica gel, 0˜5% MeOH in DCM), followed by prep-HPLC to afford (S)-N¹-(2-((1-(4-chloro-2-fluorophenyl)pyrrolidin-3-yl)(methyl)amino)phenyl)-N⁴,N⁴-dimethylbenzene-1,4-disulfonamide (comp. 110, 25 mg, 13%) as a white solid. LC/MS ESI m/z: 567 (M+H)⁺. ¹H NMR (400 MHz, DMSO) δ 9.46 (s, 1H), 8.12 (d, J=8.5 Hz, 2H), 7.95 (d, J=8.4 Hz, 2H), 7.45-7.40 (m, 1H), 7.40-7.30 (m, 1H), 7.26 (dd, J=13.5, 2.5 Hz, 1H), 7.20-7.13 (m, 2H), 7.12 (dd, J=8.7, 2.0 Hz, 1H), 6.76-6.68 (m, 1H), 3.82-3.66 (m, 1H), 3.44-3.41 (m, 1H), 3.37-3.33 (m, 2H), 3.21-3.15 (m, 1H), 2.66 (s, 6H), 2.33 (s, 3H), 1.97-1.86 (m, 1H), 1.77-1.67 (m, 1H).

The following compounds were prepared by the procedure similar to Example 10 from the corresponding amines and aryl halogens and sulfonyl chlorides.

| Compound | Structure | Name | LCMS and ¹H NMR |
|---|---|---|---|
| 111 | 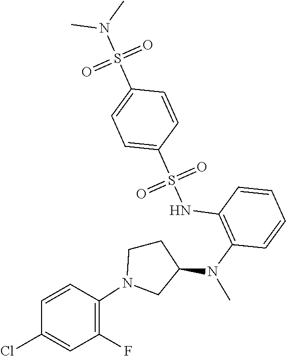 [SMILES: CN(c1ccccc1NS(=O)(=O)c1ccc(S(=O)(=O)N(C)C)cc1)[C@@H]1CCN(c2ccc(Cl)cc2F)C1] | (R)-N¹-(2-((1-(4-chloro-2- fluorophenyl)pyrrolidin-3- yl)(methyl)amino)phenyl)- N⁴,N⁴-dimethylbenzene- 1,4-disulfonamide | LC/MS ESI (m/z): 567 (M + H)⁺. 1H NMR (400 MHz, DMSO) δ 9.38 (s, 1H), 8.06 (d, J = 8.5 Hz, 2H), 7.89 (d, J = 8.4 Hz, 2H), 7.38-7.33 (m, 1H), 7.33-7.27 (m, 1H), 7.20 (dd, J = 13.5, 2.4 Hz, 1H), 7.14-7.07 (m, 2H), 7.05 (dd, J = 8.7, 2.0 Hz, 1H), 6.65 (t, J = 9.4 Hz, 1H), 3.70-3.61 (m, 1H), 3.38-3.34 (m, 1H), 3.30-3.27 (m, 2H), 3.12 (t, J = 7.3 Hz, 1H), 2.59 (s, 6H), 2.26 (s, 3H), 1.89- 1.81 (m, 1H), 1.70-1.62 (m, 1H). |
| 112 | 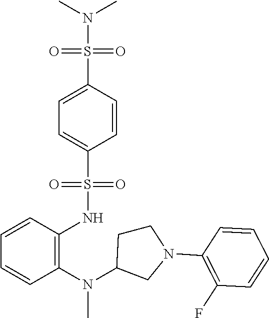 [SMILES: CN(c1ccccc1NS(=O)(=O)c1ccc(S(=O)(=O)N(C)C)cc1)C1CCN(c2ccccc2F)C1] | N¹-(2-((1-(2-Fluorophen- yl)pyrrolidin-3-yl)(meth- yl)amino)phenyl)-N⁴,N⁴- dimethylbenzene-1,4- disulfonamide | LC/MS ESI (m/z): 533 (M + H)⁺. 1 H NMR (400 MHz, CDCl3) δ 8.50 (s, 1H), 7.97 (d, J = 8.5 Hz, 2H), 7.75 (d, J = 8.5 Hz, 2H), 7.55 (dd, J = 8.1, 1.2 Hz, 1H), 7.20-7.12 (m, 2H), 7.07 (td, J = 7.6, 1.5 Hz, 1H), 7.02-6.95 (m, 2H), 6.77-6.70 (m, 1H), 6.60 (t, J = 7.8 Hz, 1H), 3.64-3.56 (m, 1H), 3.52-3.43 (m, 1H), 3.40- 3.32 (m, 2H), 3.17-3.06 (m, 1H), 2.69 (s, 6H), 2.39 (s, 3H), 2.02- 1.94 (m, 1H), 1.75-1.66 (m, 1H). |
| 113 | 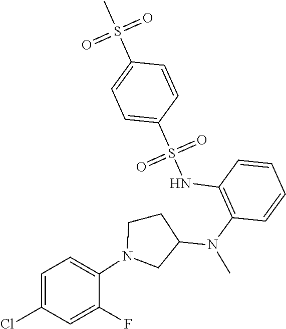 [SMILES: CN(c1ccccc1NS(=O)(=O)c1ccc(S(C)(=O)=O)cc1)C1CCN(c2ccc(Cl)cc2F)C1] | N-(2-((1-(4-Chloro-2- fluorophenyl)pyrrolidin-3- yl)(methyl)amino)phenyl)- 4-(methylsulfonyl)benzene- sulfonamide | LC/MS ESI (m/z): 538 (M + H)⁺. 1 H NMR (400 MHz, CDCl3) δ 8.46 (s, 1H), 8.02 (d, J = 8.6 Hz, 2H), 7.96 (d, J = 8.6 Hz, 2H), 7.55 (d, J = 7.0 Hz, 1H), 7.20- 7.13 (m, 2H), 7.10-7.05 (m, 1H), 7.03-6.95 (m, 2H), 6.49 (t, J = 9.0 Hz, 1H),3.62-3.54 (m, 1H), 3.47-3.38 (m, 1H), 3.37-3.28 (m, 2H), 3.13-3.05 (m, 1H), 3.04 (s, 3H), 2.39 (s, 3H), 2.03-1.93 (m, 1H), 1.75-1.63 (m, 1H). |

### Example 14. tert-Butyl 3-((2-((4-(N,N-dhnethylsulfamoyl)phenyl)sulfonamido)phenyl)(methyl)amino)pyrroidine-1-carboxylate (Compound 114)

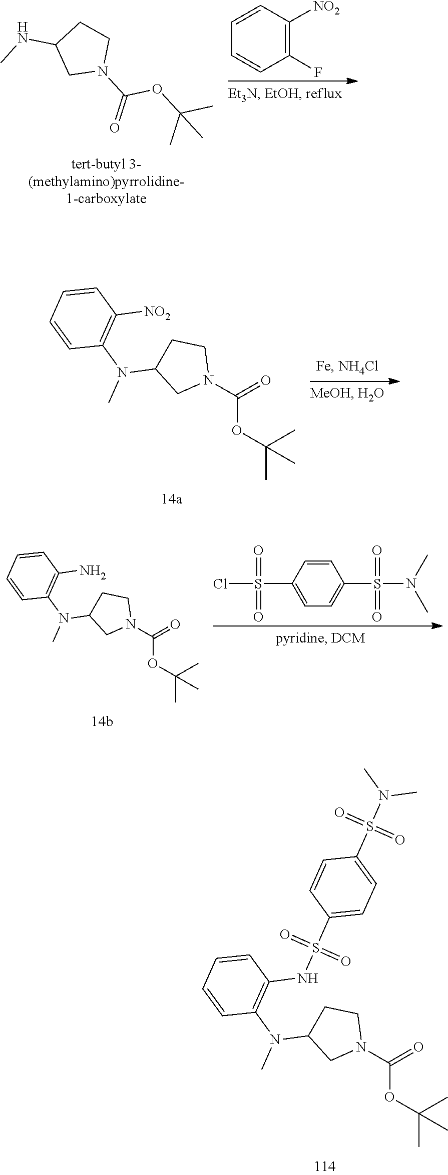 [SMILES: CNC1CCN(C(=O)OC(C)(C)C)C1 ; O=[N+]([O-])c1ccccc1F ; CN(c1ccccc1[N+](=O)[O-])C1CCN(C(=O)OC(C)(C)C)C1 ; CN(c1ccccc1N)C1CCN(C(=O)OC(C)(C)C)C1 ; CN(C)S(=O)(=O)c1ccc(S(=O)(=O)Cl)cc1 ; CN(c1ccccc1NS(=O)(=O)c1ccc(S(=O)(=O)N(C)C)cc1)C1CCN(C(=O)OC(C)(C)C)C1]

### Compound 14a. tert-Butyl 3-(methyl(2-nitrophenyl)amino)pyrrolidine-1-carboxylate

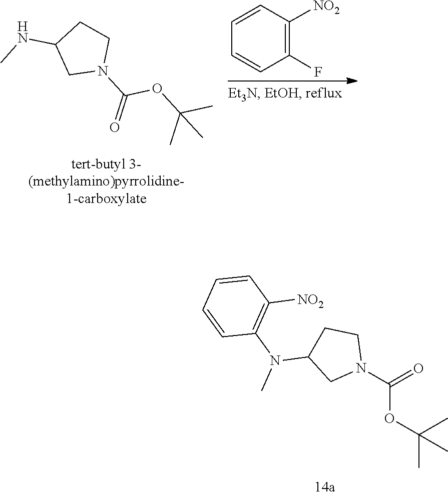 [SMILES: CNC1CCN(C(=O)OC(C)(C)C)C1 ; O=[N+]([O-])c1ccccc1F ; CN(c1ccccc1[N+](=O)[O-])C1CCN(C(=O)OC(C)(C)C)C1]

To a solution of tert-butyl 3-(methylamino)pyrrolidine-1-carboxylate (500 mg, 2.5 mmol) in EtOH (20 mL) was added 1-fluoro-2-nitrobenzene (423 mg, 3.0 mmol) and Et₃N (0.69 mL, 5 mmol), the resulting mixture was heated to reflux overnight. After being cooled down to room temperature, solvent was removed, the residue was purified by flash chromatography (silica gel, 0˜30% ethyl acetate in petroleum ether) to afford tert-butyl 3-(methyl(2-nitrophenyl)amino)pyrrolidine-1-carboxylate (comp. 14a, 230 mg, 29%) as an oil. LC/MS ESI m/z: 322 (M+H)⁺.

### Compound 14b. tert-butyl 3-((2-aminophenyl)(methyl)amino)pyrrolidine-1-carboxylate

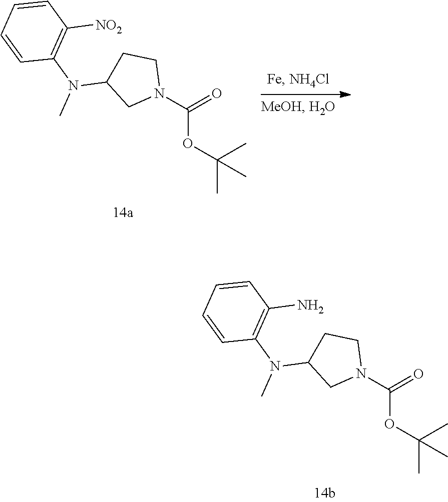 [SMILES: CN(c1ccccc1[N+](=O)[O-])C1CCN(C(=O)OC(C)(C)C)C1 ; CN(c1ccccc1N)C1CCN(C(=O)OC(C)(C)C)C1]

The mixture of tert-butyl 3-(methyl(2-nitrophenyl)amino)pyrrolidine-1-carboxylate (230 mg, 0.72 mmol), Fe (403 mg, 7.2 mmol), NH₄Cl (578 mg, 10.8 mmol) in MeOH (15 mL) and water (4 mL) was heated to 50° C. overnight. After being cooled down to room temperature and filtered, the filtrate was concentrated, residue was diluted with DCM, washed with NaHCO₃(aq.), dried over Na₂SO₄, filtered and concentrated, the residue was purified by flash chromatography (silica gel, 0˜100% ethyl acetate in petroleum ether) to afford tert-butyl 3-((2-aminophenyl)(methyl)amino)pyrrolidine-1-carboxylate (comp. 14b, 180 mg, 86%) as an oil. LC/MS ESI m/z: 292 (M+H)⁺.

### Compound 114. tert-butyl 3-((2-((4-(N,N-dimethylsulfamoyl)phenyl)sulfonamido)phenyl)(methyl)amino)pyrrolidine-1-carboxylate

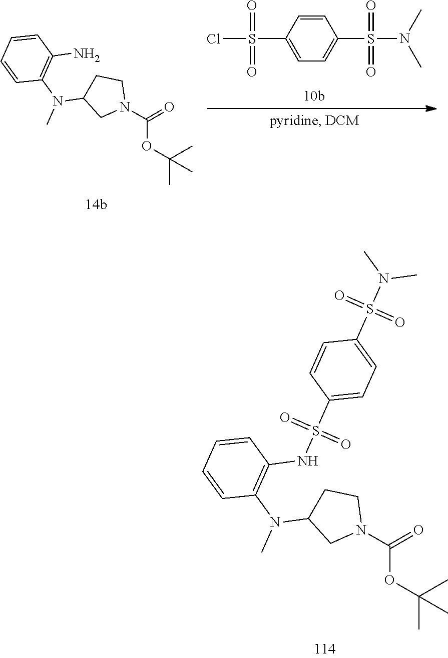 [SMILES: CN(c1ccccc1N)C1CCN(C(=O)OC(C)(C)C)C1 ; CN(C)S(=O)(=O)c1ccc(S(=O)(=O)Cl)cc1 ; CN(c1ccccc1NS(=O)(=O)c1ccc(S(=O)(=O)N(C)C)cc1)C1CCN(C(=O)OC(C)(C)C)C1]

At 0° C., to a solution of tert-butyl 3-((2-aminophenyl)(methyl)amino)pyrrolidine-1-carboxylate (180 mg, 0.62 mmol) in DCM (10 mL) was added pyridine (0.1 mL, 1.24 mmol), followed by 4-(N,N-dimethylsulfamoyl)benzenesulfonyl chloride (comp. 10b, 264 mg, 0.93 mmol), the resulting mixture was stirred at room temperature overnight. The reaction was quenched with water, extracted with DCM twice, the combined organic layer was washed with brine, dried over Na₂SO₄, filtered and concentrated. The residue was purified by flash chromatography (silica gel, 0˜5% MeOH in DCM), followed by prep-HPLC to afford tert-butyl 3-((2-((4-(N,N-dimethylsulfamoyl)phenyl)sulfonamido)phenyl)(methyl)amino)pyrrolidine-1-carboxylate (comp. 114, 80 mg, 24%) as a white solid. LC/MS ESI m/z: 539 (M+H)⁺. ¹H NMR (400 MHz, DMSO) δ 8.75 (s, 1H), 8.07 (d, J=8.5 Hz, 2H), 7.91 (d, J=8.4 Hz, 2H), 7.35 (d, J=7.6 Hz, 1H), 7.25 (d, J=6.1 Hz, 1H), 7.13-7.03 (m, 2H), 3.66-3.47 (m, 1H), 3.32-3.24 (m, 2H), 3.17-3.04 (m, 1H), 3.02-2.86 (m, 1H), 2.60 (s, 6H), 2.19 (s, 3H), 1.71 (m, 1H), 1.62-1.50 (m, 1H), 1.38 (s, 9H).

### Example 15. N¹-(2-(4-(bicyclo[1.1.1]pentan-1-yl)piperazin-1-yl)phenyl)-N⁴,N⁴-dimethylbenzene-1,4-disulfonamide ((Compound 115)

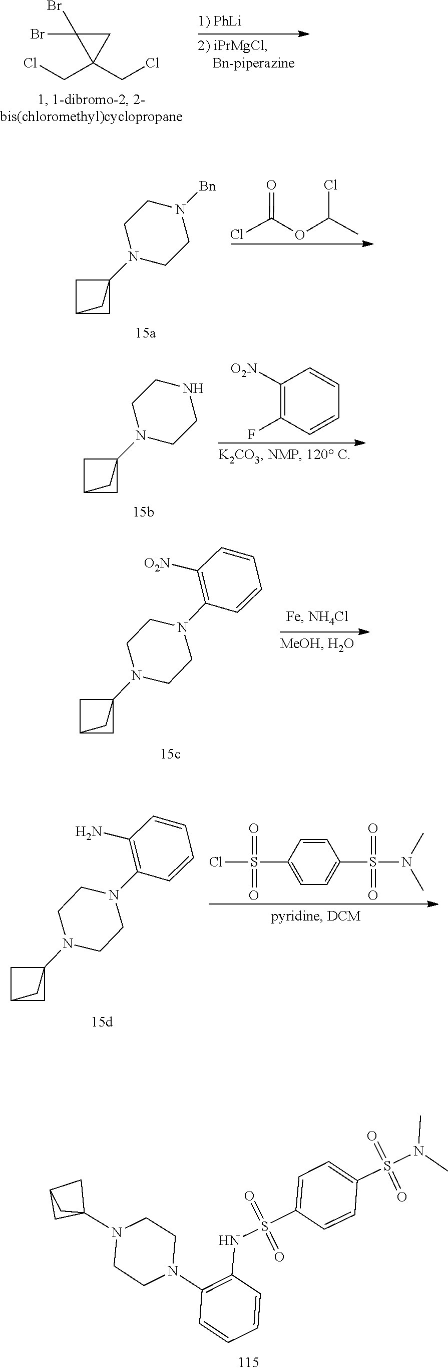

### Compound 15a. 1-benzyl-4-(bicyclo[1.1.1]pentan-1-yl)piperazine

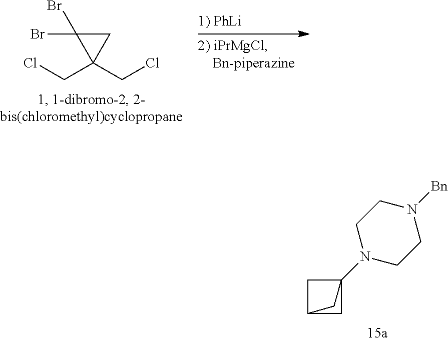 [SMILES: ClCC1(CCl)CC1(Br)Br ; c1ccc(CN2CCN(C34CC(C3)C4)CC2)cc1]

At −45° C., to a solution of 1,1-dibromo-2,2-bis(chloromethyl)cyclopropane (2.97 g, 10 mmol) in THF (3 mL) was added PhLi (20 mL, 1.0M in ethyl ether) was added slowly via syringe, the resulting mixture was stirred at the same temperature for 5 min and at ice-water bath for 2 h. A solution of 1-benzylpiperazine (3.52 g, 20 mmol), which was pre-treated with iPrMgCl·LiCl (31 mL, 1.3M in THF) carefully at room temperature, was added to the above mixture dropwise at ice-water bath. After addition, the reaction mixture was transferred to a pressure tube and heated at 45° C. overnight. After being cooled down to room temperature, the reaction was quenched with sat. aq. NH₄Cl, extracted with EtOAc twice, the combined organic layer was washed with brine, dried over Na₂SO₄, filtered and concentrated under reduced pressure. The residual solvent was removed by hi-vacuum and the crude material was purified by flash chromatography (silica gel, 0˜30% ethyl acetate in petroleum ether) to afford 1-benzyl-4-(bicyclo[1.1.1]pentan-1-yl)piperazine (comp. 15a, 1 g, as a very crude product). LC/MS ESI m/z: 243 (M+H)⁺.

### Compound 15b. 1-(bicyclo[1.1.1]pentan-1-yl)piperazine

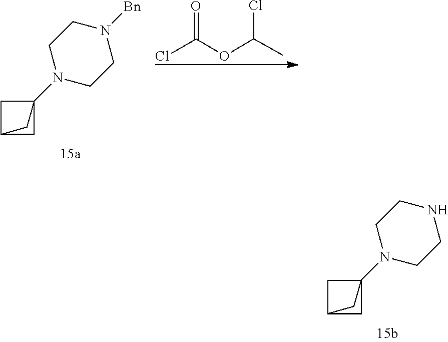

At 0° C., to a solution of 1-benzyl-4-(bicyclo[1.1.1]pentan-1-yl)piperazine (1 g, 4.13 mmol) in DCM (10 mL) was added 1-chloroethyl carbonochloridate (2.95 g, 20.65 mmol) dropwise, the resulting mixture was stirred at room temperature overnight. Solvent was removed and the residue was diluted with MeOH (20 mL) and heated to 50° C. for 2 h, After being cooled down to room temperature, MeOH was removed and the residue was diluted with DCM, washed with sat. NaHCO₃(aq.), organic aqueous layer was extracted with DCM/iPrOH (85:15) twice, the combined organic layer was dried over Na₂SO₄, filtered and concentrated. The residue was purified by flash chromatography (silica gel, 0˜20% MeOH in DCM) to afford 1-(bicyclo[1.1.1]pentan-1-yl)piperazine (comp. 15b, 630 mg, as a crude product). LC/MS ESI m/z: 153 (M+H)⁺.

Compound 15c. 1-(bicyclo[1.1.1]pentan-1-yl)-4-(2-nitrophenyl)piperazine

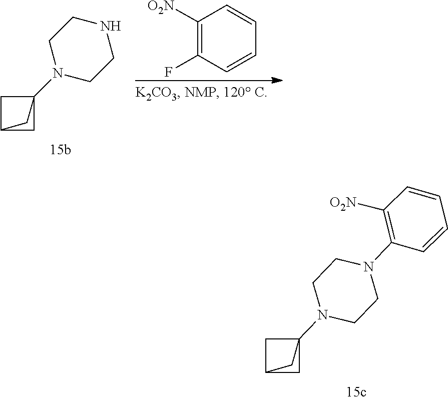

To a solution of 1-(bicyclo[1.1.1]pentan-1-yl)piperazine (630 mg, 4.14 mmol) in NMP (20 mL) was added 1-fluoro-2-nitrobenzene (700 mg, 4.97 mmol) and K₂CO₃ (1.7 g, 12.42 mmol), the resulting mixture was heated to 120° C. overnight. After being cooled down to room temperature, the reaction mixture was partitioned between EtOAc and water, organic layer was separated, aqueous layer was extracted with EtOAc twice, the combined organic layers were washed with brine, dried over Na₂SO₄, filtered and concentrated, the residue was purified by flash chromatography (silica gel, 0˜30% ethyl acetate in petroleum ether) to afford 1-(bicyclo[1.1.1]pentan-1-yl)-4-(2-nitrophenyl)piperazine (comp. 15c, 80 mg, 7%). LC/MS ESI m/z: 274 (M+H)⁺.

### Compound 15d. 2-(4-(bicyclo[1.1.1]pentan-1-yl)piperazin-1-yl)aniline

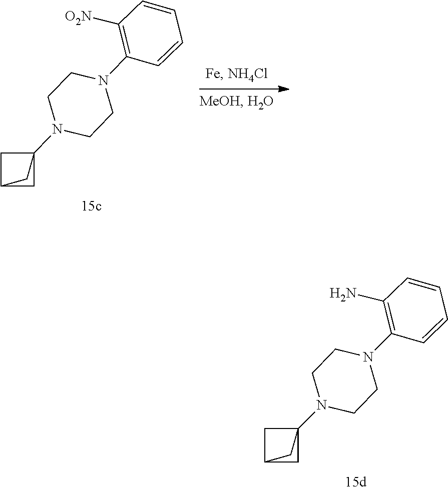

The mixture of 1-(bicyclo[1.1.1]pentan-1-yl)-4-(2-nitrophenyl)piperazine (80 mg, 0.29 mmol), Fe (162 mg, 2.9 mmol), NH₄Cl (233 mg, 4.35 mmol) in MeOH (10 mL) and water (2 mL) was heated to 50° C. overnight. After being cooled down to room temperature and filtered, the filtrate was concentrated, residue was diluted with DCM, washed with NaHCO₃(aq.), dried over Na₂SO₄, filtered and concentrated, the residue was purified by flash chromatography (silica gel, 0˜100% ethyl acetate in petroleum ether) to afford 2-(4-(bicyclo[1.1.1]pentan-1-yl)piperazin-1-yl)aniline (comp. 15d, 20 mg, 28%). LC/MS ESI m/z: 244 (M+H)⁺.

### Compound 115. N¹-(2-(4-(bicyclo[1.1.1]pentan-1-yl)piperazin-1-yl)phenyl)-N⁴,N⁴-dimethylbenzene-1,4-disulfonamide

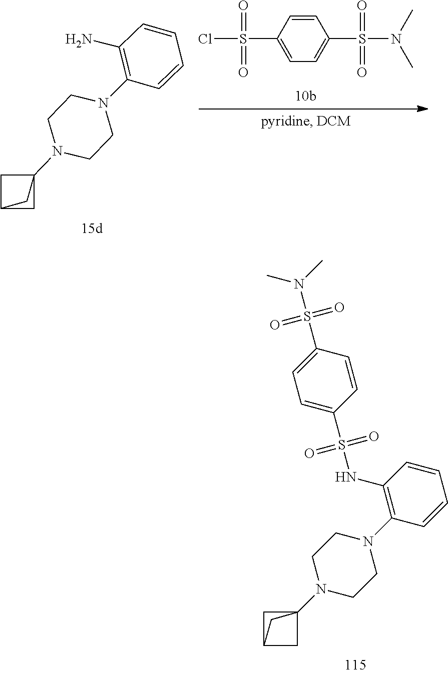

At 0° C., to a solution of 2-(4-(bicyclo[1.1.1]pentan-1-yl)piperazin-1-yl)aniline (20 mg, 0.082 mmol) in DCM (8 mL) was added pyridine (13 mg, 0.16 mmol), followed by 4-(N,N-dimethylsulfamoyl)benzenesulfonyl chloride (35 mg, 0.12 mmol), the resulting mixture was stirred at room temperature overnight. The reaction was quenched with sat. NaHCO₃(aq.), extracted with DCM twice, the combined organic layers were washed with brine, dried over Na₂SO₄, filtered and concentrated. The residue was purified by flash chromatography (silica gel, 0˜5% MeOH in DCM), followed by prep-HPLC to afford N¹-(2-(4-(bicyclo[1.1.1]pentan-1-yl)piperazin-1-yl)phenyl)-N⁴,N¹-dimethylbenzene-1,4-disulfonamide (comp. 115, 15 mg, 37%) as a white solid. LC/MS ESI m/z: 491 (M+H)⁺. ¹H NMR (400 MHz, DMSO) δ 9.25 (s, 1H), 8.01 (d, J=8.4 Hz, 2H), 7.93 (d, J=8.5 Hz, 2H), 7.24-7.18 (m, 1H), 7.17-7.11 (m, 2H), 7.09-7.02 (m, 1H), 2.63 (s, 6H), 2.60-2.55 (m, 4H), 2.43 (br, 1H), 2.41-2.32 (4H), 1.72 (br, 6H).

### Example 16. trans-N¹-2-(4-(2-Fluorophenyl)piperazin-1-yl)cyclohexyl)-N⁴,N⁴-dimethylbenzene-1,4-disulfonamide (Compound 116)

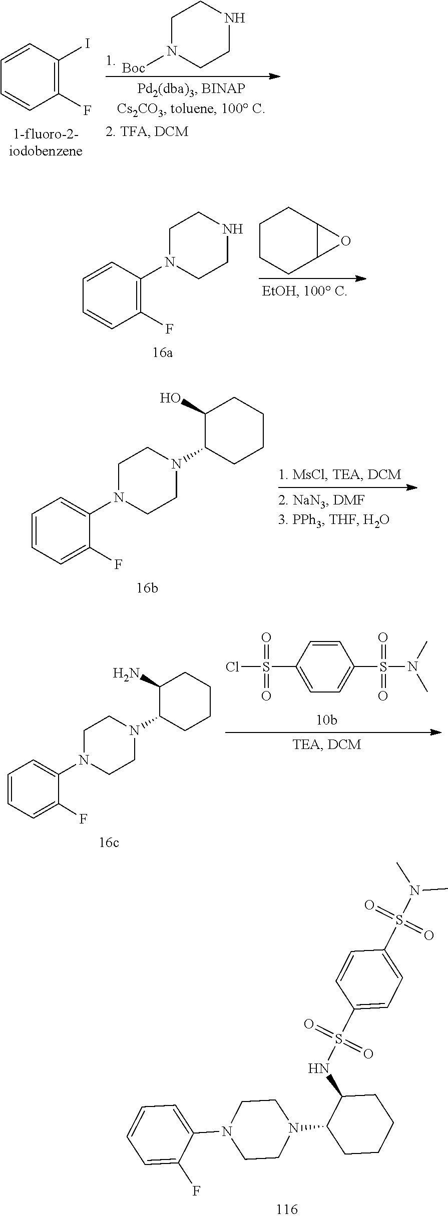

### Compound 16a. 1-(2-fluorophenyl)piperazine

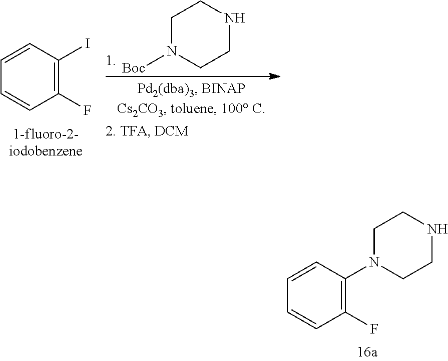 [SMILES: Fc1ccccc1I ; CC(C)(C)OC(=O)N1CCNCC1 ; Fc1ccccc1N1CCNCC1]

To a solution of tert-butyl piperazine-1-carboxylate (402 mg, 2.16 mmol) in toluene (20 mL) was added 1-fluoro-2-iodobenzene (400 mg, 1.8 mmol), Pd₂(dba)₃ (165 mg, 0.18 mmol), BINAP (168 mg, 0.27) and Cs₂CO₃ (1.77 g, 5.4 mmol), the resulting mixture was heated to 100° C. overnight. After being cooled down to room temperature, solvent was removed and the residue was purified by flash chromatography (silica gel, 0-50% ethyl acetate in petroleum ether) to afford tert-butyl 4-(2-fluorophenyl)piperazine-1-carboxylate (230 mg, 82%). LC/MS ESI m/z: 281 (M+H)⁺.

At 0° C., to a solution of tert-butyl 4-(2-fluorophenyl)piperazine-1-carboxylate (230 mg, 0.82 mmol) in DCM (10 mL) was added TFA (2 mL), the resulting mixture was stirred at room temperature for 2 h. After removal of solvent, the residue was diluted with DCM, washed with NaHCO₃(aq.), the organic layer was extracted with DCM twice, the combined organic layers were washed with brine, dried over Na₂SO₄, filtered and concentrated. The residue was used in the next step directly. Compound 16a, LC/MS ESI m/z: 181 (M+H)⁺.

### Compound 16b. trans-2-(4-(2-fluorophenyl)piperazin-1-yl)cyclohexan-1-ol

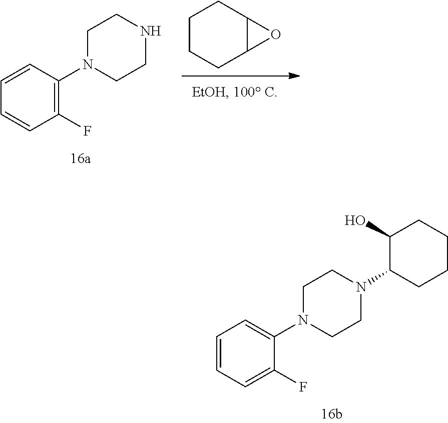

To a solution of 1-(2-fluorophenyl)piperazine (140 mg, 0.78 mmol) in EtOH (10 mL) was added 7-oxabicyclo[4.1.0]heptane (766 mg, 7.8 mmol), the resulting mixture was heated to reflux overnight. After being cooled down to room temperature, solvent was removed, the residue was purified by flash chromatography (silica gel, 0˜30% ethyl acetate in petroleum ether) to afford trans-2-(4-(2-fluorophenyl)piperazin-1-yl)cyclohexan-1-ol (comp. 16b, 150 mg, 69%) as an oil. LC/MS ESI m/z: 279 (M+H)⁺.

### Compound 16c. trans-2-(4-(2-fluorophenyl)piperazin-1-yl)cyclohexan-1-amine

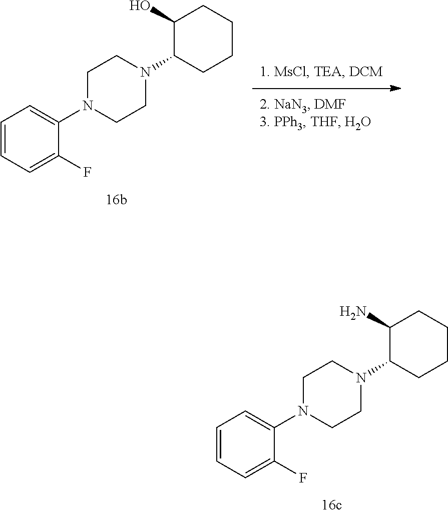 [SMILES: O[C@H]1CCCC[C@@H]1N1CCN(c2ccccc2F)CC1 ; N[C@H]1CCCC[C@@H]1N1CCN(c2ccccc2F)CC1]

At 0° C., to a solution of trans-2-(4-(2-fluorophenyl)piperazin-1-yl)cyclohexan-1-ol (150 mg, 0.54 mmol) in DCM (5 mL) was added TEA (109 mg, 1.08 mmol), followed by MsCl (74 mg, 0.65 mmol) in DCM (2 mL) dropwise. The resulting mixture was stirred at the same temperature for 1 h, then was portioned between DCM and water, organic layer was separated, aqueous layer was extracted with DCM twice, the combined organic layers were washed with brine, dried over Na₂SO₄, filtered and concentrated. The residue was used to the next step directly. 160 mg, 83% yield. LC/MS ESI m/z: 357 (M+H)⁺.

To a solution of Trans-2-(4-(2-fluorophenyl)piperazin-1-yl)cyclohexyl methanesulfonate (160 mg, 0.45 mmol) in DMF (6 mL) was added NaN₃ (292 mg, 4.5 mmol), the resulting mixture was heated to 80° C. overnight. After being cooled down to room temperature, the reaction was partitioned between EtOAc and water, organic layer was separated, aqueous layer was extracted with EtOAc twice, the combined organic layers were washed with brine, dried over Na₂SO₄, filtered and concentrated, the residue was purified by flash chromatography (silica gel, 0˜20% ethyl acetate in petroleum ether) to afford trans-1-(2-azidocyclohexyl)-4-(2-fluorophenyl)piperazine (100 mg, 73%). LC/MS ESI m/z: 304 (M+H)⁺.
The mixture of trans-1-(2-azidocyclohexyl)-4-(2-fluorophenyl)piperazine (100 mg, 033 mmol), PPh₃ (173 mg, 0.66 mmol) in THF (10 mL) and water (3 mL) was heated to 60° C. overnight. After being cooled down to room temperature, the reaction was partitioned between EtOAc and water, organic layer was separated, aqueous layer was extracted with EtOAc twice, the combined organic layers were washed with brine, dried over Na₂SO₄, filtered and concentrated, the residue was purified by flash chromatography (silica gel, 0˜100% ethyl acetate in petroleum ether) to afford trans-2-(4-(2-fluorophenyl)piperazin-1-yl)cyclohexan-1-amine (comp. 16c, 60 mg, 66%). LC/MS ESI m/z: 278 (M+H)⁺.

### Compound 116. trans-N¹-(2-(4-(2-Fluorophenyl)piperazin-1-yl)cyclohexyl)-N⁴,N⁴-dimeth ylbenzene-1,4-disulfonamide

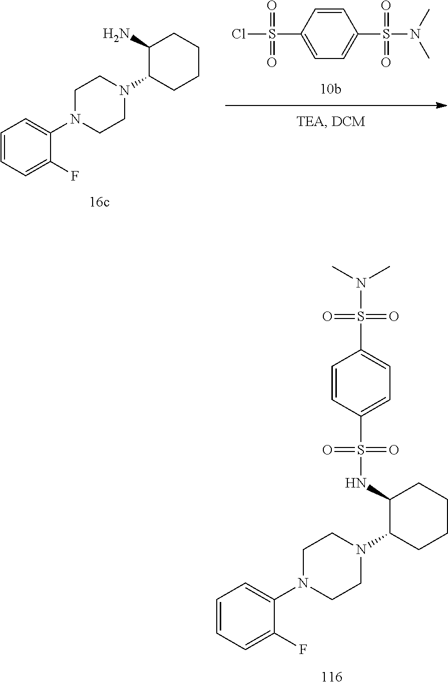 [SMILES: N[C@H]1CCCC[C@@H]1N1CCN(c2ccccc2F)CC1 ; CN(C)S(=O)(=O)c1ccc(S(=O)(=O)Cl)cc1 ; CN(C)S(=O)(=O)c1ccc(S(=O)(=O)N[C@H]2CCCC[C@@H]2N2CCN(c3ccccc3F)CC2)cc1]

At 0° C., to a solution of trans-2-(4-(2-fluorophenyl)piperazin-1-yl)cyclohexan-1-amine (60 mg, 0.22 mmol) in DCM (8 mL) was added pyridine (35 mg, 0.44 mmol), followed by 4-(N,N-dimethylsulfamoyl)benzenesulfonyl chloride (comp. 10b, 94 mg, 0.33 mmol), the resulting mixture was stirred at room temperature overnight. The reaction was quenched with water, extracted with DCM twice, the combined organic layers were washed with brine, dried over Na₂SO₄, filtered and concentrated. The residue was purified by flash chromatography (silica gel, 0˜5% MeOH in DCM), followed by prep-HPLC to afford trans-N¹-(2-(4-(2-fluorophenyl)piperazin-1-yl)cyclohexyl)-N⁴,N⁴-dimethylbenzene-1,4-disulfonamide (comp. 116, 33 mg, 29%) as a white solid. LC/MS ESI m/z: 525 (M+H)⁺. ¹H NMR (400 MHz, CDCl₃) δ 8.09 (d, J=8.4 Hz, 2H), 7.92 (d, J=8.4 Hz, 2H), 7.13-7.08 (m, 1H), 7.07-6.95 (m, 3H), 3.46-2.90 (m, 7H), 2.88-2.67 (m, 9H), 2.11-1.96 (m, 2H), 1.90-1.85 (m, 1H), 1.72-1.66 (m, 1H), 1.35-1.18 (m, 4H).

The following compound was prepared by the procedure similar to Example 16 from the corresponding aryl halogen.

| Compound | Structure | Name | LCMS and ¹H NMR |
|---|---|---|---|
| 117 | 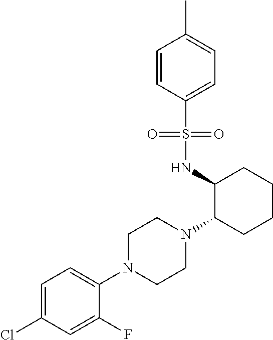 [SMILES: Cc1ccc(S(=O)(=O)N[C@H]2CCCC[C@@H]2N2CCN(c3ccc(Cl)cc3F)CC2)cc1] | trans-N-(2-(4-(4- chloro-2-fluoro- phenyl)piperazin- 1-yl)cyclohexyl)- 4-methylbenzene- sulfonamide | LC/MS ESI (m/z): 466 (M + H)+. 1 H NMR (400 MHz, CDCl₃) δ 7.78 (d, J = 8.2 Hz, 2H), 7.29 (d, J = 8.1 Hz, 2H), 7.12-7.05 (m, 2H), 6.91 (t, J = 9.0 Hz, 1H), 3.64-2.80 (m, 10H), 2.42 (s, 3H), 2.08 (d, J = 9.4 Hz, 1H), 1.89 (d, J =12.5 Hz, 1H), 1.85-1.78 (m, 1H), 1.67 (d, J = 13.5 Hz, 1H), 1.38-1.16 (m, 4H). |

### Example 18. Synthesis of trans-N¹-(2-(((R)-1-(4-chloro-2-fluorophenyl)pyrrolidin-3-yl)(methyl)amino)cyclohexyl)-N⁴,N⁴-dimethylbenzene-1,4-disulfonamide (Compound 118)

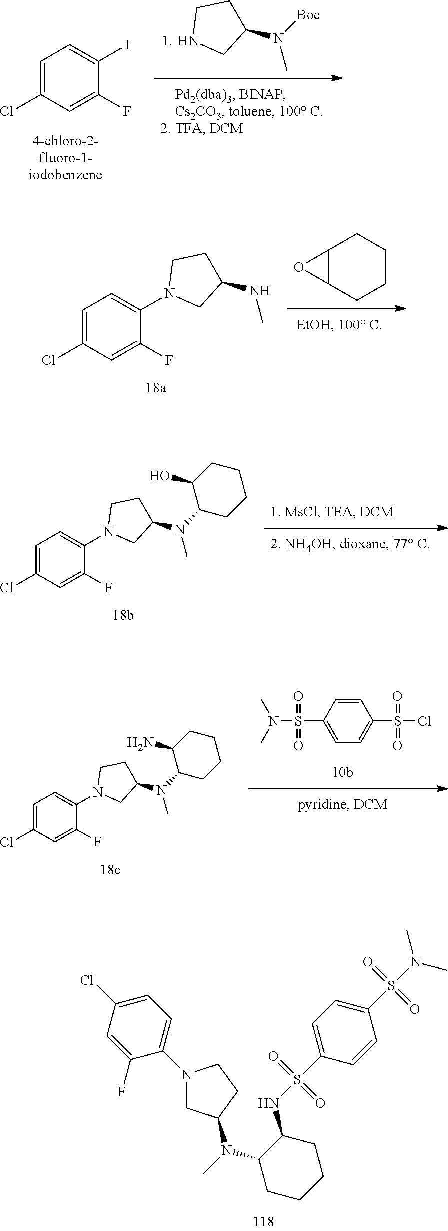

### Compound 18a. (R)-1-(4-chloro-2-fluorophenyl)-N-methylpyrrolidin-3-amine

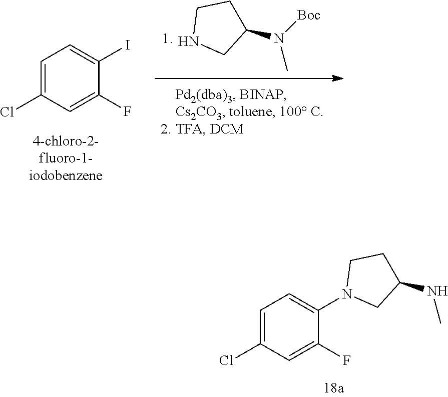

To a solution of tert-butyl (R)-methyl(pyrrolidin-3-yl)carbamate (260 mg, 1.3 mmol) in toluene (15 mL) was added 4-chloro-2-fluoro-1-iodobenzene (333 mg, 1.3 mmol), Pd₂(dba)₃ (119 mg, 0.13 mmol), BINAP (121 mg, 0.27) and CS₂CO₃ (1.28 g, 3.9 mmol), the resulting mixture was heated to 100° C. overnight. After being cooled down to room temperature, solvent was removed and the residue was purified by flash chromatography (silica gel, 0˜40% ethyl acetate in petroleum ether) to afford tert-butyl (R)-(1-(4-chloro-2-fluorophenyl)pyrrolidin-3-yl)(methyl)carbamate (340 mg, 79%). LC/MS ESI m/z: 329 (M+H)⁺.

At 0° C., to a solution of tert-butyl (R)-(1-(4-chloro-2-fluorophenyl)pyrrolidin-3-yl)(methyl)carbamate (340 mg, 1.04 mmol) in DCM (10 mL) was added TFA (2 mL), the resulting mixture was stirred at room temperature for 2 h. After removal of solvent, the residue was diluted with DCM, washed with NaHCO₃(aq.), the organic layer was extracted with DCM twice, the combined organic layers were washed with brine, dried over Na₂SO₄, filtered and concentrated. The residue was used to the next step directly. Compound 18a, LC/MS ESI m/z: 229 (M+H)⁺.

### Compound 18b. trans-2-(((R)-1-(4-chloro-2-fluorophenyl)pyrrolidin-3-yl)(methyl)amino) cyclohexan-1-ol

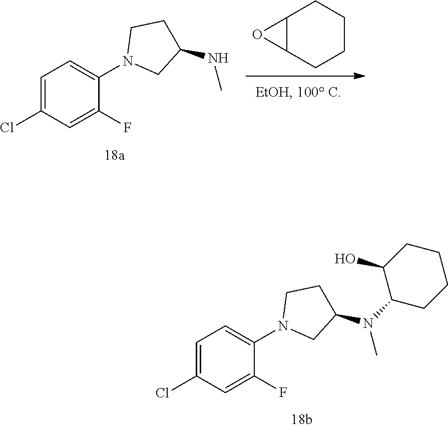 [SMILES: CN[C@@H]1CCN(c2ccc(Cl)cc2F)C1 ; C1CCC2OC2C1 ; CN([C@@H]1CCN(c2ccc(Cl)cc2F)C1)[C@H]1CCCC[C@@H]1O]

To a solution of (R)-1-(4-chloro-2-fluorophenyl)-N-methylpyrrolidin-3-amine (220 mg, 0.96 mmol) in EtOH (10 mL) was added 7-oxabicyclo[4.1.0]heptane (942 mg, 9.6 mmol), the resulting mixture was heated to reflux for 3 days. After being cooled down to room temperature, solvent was removed, the residue was purified by flash chromatography (silica gel, 0˜30% ethyl acetate in petroleum ether) to afford trans-2-(((R)-1-(4-chloro-2-fluorophenyl)pyrrolidin-3-yl)(methyl)amino)cyclohexan-1-ol (comp. 18b, 260 mg, 83%). LC/MS ESI m/z: 327 (M+H)⁺.

### Compound 18c. trans-N¹-((R)-1-(4-chloro-2-fluorophenyl)pyrrolidin-3-yl)-N¹-methylcyclohexane-1,2-diamine

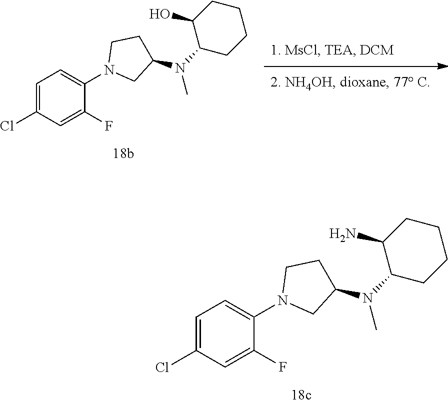 [SMILES: CN([C@@H]1CCN(c2ccc(Cl)cc2F)C1)[C@H]1CCCC[C@@H]1O ; CN([C@@H]1CCN(c2ccc(Cl)cc2F)C1)[C@H]1CCCC[C@@H]1N]

At 0° C., to a solution of trans-2-(((R)-1-(4-chloro-2-fluorophenyl)pyrrolidin-3-yl)(methyl)amino)cyclohexan-1-ol (260 mg, 0.79 mmol) in DCM (5 mL) was added TEA (161 mg, 1.59 mmol), followed by MsCl (135 mg, 1.19 mmol) in DCM (2 mL) dropwise. The resulting mixture was stirred at the same temperature for 1 h, then was portioned between DCM and water, organic layer was separated, aqueous layer was extracted with DCM twice, the combined organic layers were washed with brine, dried over Na₂SO₄, filtered and concentrated. The residue was used to the next step directly. 320 mg, 100% yield. LC/MS ESI m/z: 405 (M+H)⁺.

To a solution of trans-2-(((R)-1-(4-chloro-2-fluorophenyl)pyrrolidin-3-yl)(methyl)amino)cyclohexyl methanesulfonate (320 mg, 0.79 mmol) in dioxane (15 mL) was added ammonium hydroxide (5 mL), the resulting mixture was heated to 77° C. overnight. After being cooled down to room temperature, solvent was removed, the residue was purified by flash chromatography (silica gel, 0˜100% ethyl acetate in petroleum ether) to afford trans-N¹-((R)-1-(4-chloro-2-fluorophenyl)pyrrolidin-3-yl)-N¹-methylcyclohexane-1,2-diamine (comp. 18c, 160 mg, 62%). LC/MS ESI m/z: 326 (M+H)⁺.

### Compound 118. trans-N¹-(2-(((R)-1-(4-Chloro-2-fluorophenyl)pyrrolidin-3-yl)(methyl)amino)cyclohexyl)-N⁴,N⁴-dimethylbenzene-1,4-disulfonamide

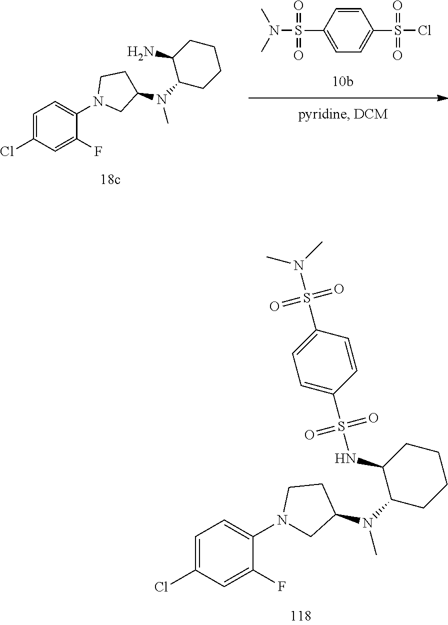 [SMILES: CN([C@@H]1CCN(c2ccc(Cl)cc2F)C1)[C@H]1CCCC[C@@H]1N ; CN(C)S(=O)(=O)c1ccc(S(=O)(=O)Cl)cc1 ; CN([C@@H]1CCN(c2ccc(Cl)cc2F)C1)[C@H]1CCCC[C@@H]1NS(=O)(=O)c1ccc(S(=O)(=O)N(C)C)cc1]

At 0° C., to a solution of trans-N¹-((R)-1-(4-chloro-2-fluorophenyl)pyrrolidin-3-yl)-N¹-methylcyclohexane-1,2-diamine (160 mg, 0.49 mmol) in DCM (15 mL) was added pyridine (77 mg, 0.98 mmol), followed by 4-(N,N-dimethylsulfamoyl)benzenesulfonyl chloride (comp. 10b, 209 mg, 0.74 mmol), the resulting mixture was stirred at room temperature overnight. The reaction was quenched with sat. NaHCO₃ (aq.), extracted with DCM twice, the combined organic layers were washed with brine, dried over Na₂SO₄, filtered and concentrated. The residue was purified by flash chromatography (silica gel, 0˜5% MeOH in DCM), followed by prep-HPLC to afford trans-N¹-(2-(((R)-1-(4-chloro-2-fluorophenyl)pyrrolidin-3-yl)(methyl)amino)cyclohexyl)-N⁴,N⁴-dimethylbenzene-1,4-disulfonamide (comp. 118.25 mg, 9%) as a white solid. LC/MS ESI m/z: 573 (M+H)⁺. ¹H NMR (400 MHz, MeOD) δ 8.12 (dd, J=8.5, 3.1 Hz, 2H), 7.97-7.92 (m, 2H), 7.01 (dd, J=17.0, 5.1 Hz, 2H), 6.67 (dd, J=17.3, 9.1 Hz, 1H), 3.37 (t, 3H), 3.12 (t, 3H), 2.70 (d, J=2.9 Hz, 6H), 2.45 (t, 1H), 2.18 (t, 1H), 2.03 (t, J=28.8, 11.5, 6.1 Hz, 1H), 1.74 (t, 7H), 1.29 (t, 4H).

The following compounds were prepared by the procedure similar to the synthesis of compound 118 from the corresponding amines and epoxides.

| Compound | Structure | Name | LCMS and ¹H NMR |
|---|---|---|---|
| 119 | 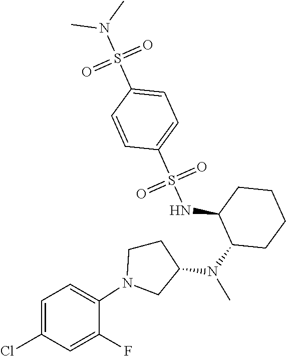 [SMILES: CN([C@H]1CCN(c2ccc(Cl)cc2F)C1)[C@H]1CCCC[C@@H]1NS(=O)(=O)c1ccc(S(=O)(=O)N(C)C)cc1] | trans-N¹-(2-(((S)-1-(4- chloro-2-fluorophenyl)- pyrrolidin-3-yl)(methyl)- amino)cyclohexyl)- N⁴,N⁴-dimethylbenzene- 1,4-disulfonamide | LC/MS ESI (m/z): 573 (M + H)⁺. 1 H NMR (400 MHz, MeOD) δ 8.14-8.09 (m, 2H), 7.97 - 7.92 (m, 2H), 7.06-6.96 (m, 2H), 6.72-6.64 (m, 1H), 3.43-3.33 (m, 3H), 3.24- 2.97 (m, 3H), 2.70 (d, J = 2.8 Hz, 6H), 2.53-2.33 (m,1H), 2.20-2.12 (m, 1H), 2.08-1.96 (m, 1H), 1.85- 1.62 (m, 7H), 1.28-1.15 (m, 4H). |
| 120 | 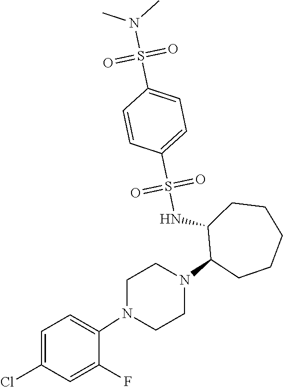 [SMILES: CN(C)S(=O)(=O)c1ccc(S(=O)(=O)N[C@@H]2CCCCC[C@H]2N2CCN(c3ccc(Cl)cc3F)CC2)cc1] | trans-N¹-(2-(4-(4-chloro- 2-fluorophenyl)piperazin- 1-yl)cycloheptyl)-N⁴,N⁴ dimethylbenzene-1,4- disulfonamide | LC/MS ESI (m/z): 573 (M + H)⁺. 1 H NMR (400 MHz, CDCl₃) δ 8.08 (d, J = 8.3 Hz, 2H), 7.92 (d, J = 8.5 Hz, 2H), 7.10-7.01 (m, 2H), 6.83 (t, J = 8.8 Hz, 1H), 3.08- 3.01 (m, 1H), 3.01-2.77 (m, 4H), 2.75 (s, 6H), 2.67-2.35 (m, 4H), 2.35-2.27 (m, 1H), 2.20-2.10 (m, 1H), 1.91-1.84 (m, 1H), 1.72-1.60 (m, 3H), 1.49-1.28 (m, 5H). |
| 121 | 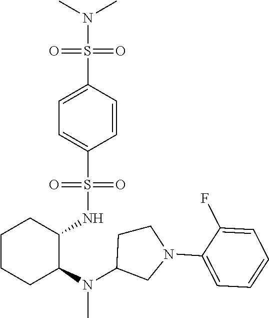 [SMILES: CN(C1CCN(c2ccccc2F)C1)[C@H]1CCCC[C@@H]1NS(=O)(=O)c1ccc(S(=O)(=O)N(C)C)cc1] | trans-N¹-(2-((1-(2- fluorophenyl)pyrrolidin- 3-yl)(methyl)amino)- cyclohexyl)-N⁴,N⁴- dimethylbenzene-1,4- disulfonamide | LC/MS ESI (m/z): 539 (M + H)⁺. 1 H NMR (400 MHz, CDCl₃) δ 8.07 (d, J = 8.4 Hz, 2H), 7.92 (dd, J = 8.5, 1.9 Hz, 2H), 7.02-6.96 (m, 2H), 6.76-6.60 (m, 2H), 6.09 (br, 1H), 3.48-3.41 (m, 3H), 3.30-3.18 (m, 2H), 2.93-2.82 (m,1H), 2.74 (d, J = 3.6 Hz, 6H), 2.33-2.28 (m, 1H), 2.13-2.07 (m, 1H), 1.93-1.72 (m, 7H), 1.69-1.65 (m, 1H), 1.24- 1.15 (m, 4H). |

### Example 22. cis-N¹-((1R,2S)-2-(4-(4-chloro-2-fluorophenyl)piperazin-1-yl)cycloheptyl)-N⁴,N⁴-dimethylbenzene-1,4-disulfonamide (Compound 122)

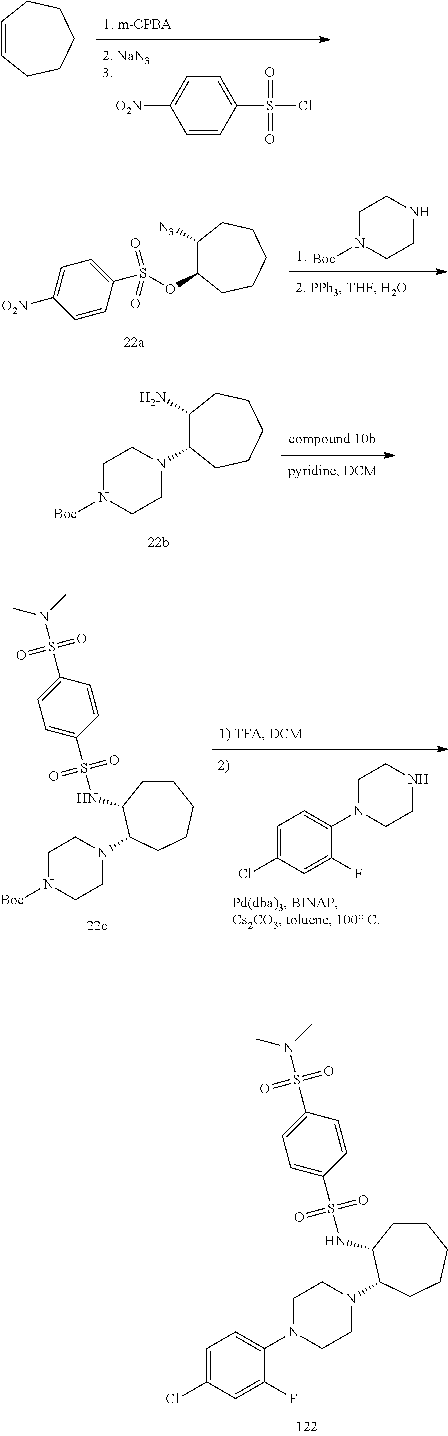

### Compound 22a. trans-2-Azidocycloheptyl 4-nitrobenzenesulfonate

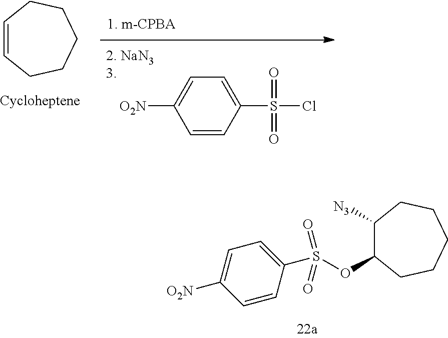 [SMILES: C1=CCCCCC1 ; O=[N+]([O-])c1ccc(S(=O)(=O)Cl)cc1 ; [N-]=[N+]=N[C@@H]1CCCCC[C@H]1OS(=O)(=O)c1ccc([N+](=O)[O-])cc1]

At 0° C., to a solution of cycloheptene (1.7 g, 17.68 mmol) in DCM (18 mL) 0.3 M aq. NaHCO₃ (141 mL) was added m-CPBA (4.07 g, 23.57 mmol) in portions. The resulting mixture was stirred at room temperature overnight. The reaction was extracted with DCM twice, the combined organic layers were washed with brine, dried over Na₂SO₄, filtered and concentrated. The residue was purified by flash chromatography (silica gel, 0˜25% ethyl acetate in petroleum) to afford 8-oxabicyclo[5.1.0]octane (1.5 g, 76%) as a light yellow liquid.

To a mixture of 8-oxabicyclo[5.1.0]octane (560 mg, 4.99 mmol), NH4Cl (534 mg, 9.98 mmol) in CH3OH (16 mL) and H2O (2 mL) was added NaN3 (3.25 g, 50 mmol). The reaction mixture was degassed and stirred at room temperature for 24 hr. The reaction mixture was then concentrated to 1/10 its volume, diluted with water (20 mL) and extracted with EtOAc twice. The combined organic layers were washed with brine, dried over Na2SO4, filtered, concentrated and chromatographed (0-40% ethyl acetate in petroleum ether) to provide trans-2-azidocycloheptan-1-ol (340 mg, 44%).

At 0° C., to a solution of trans-2-azidocycloheptan-1-ol (340 mg, 2.19 mmol) in MeCN (10 mL) was added pyridine (433 mg, 5.48 mmol), followed by 4-nitrobenzenesulfonyl chloride (971 mg, 4.38 mmol) in portions. The resulting mixture was stirred at room temperature overnight. The reaction was quenched with ice water extracted with DCM twice, the combined organic layers were washed with 1 N HCl, and saturated NaHCO₃, dried and concentrated. The residue was purified by flash chromatography (silica gel, 0˜60% ethyl acetate in petroleum ether) to afford trans-2-azidocycloheptyl 4-nitrobenzenesulfonate (comp. 22a, 422 mg, 57%).

### Compound 22b. cis-tert-Butyl 4-(2-aminocycloheptyl)piperazine-1-carboxylate

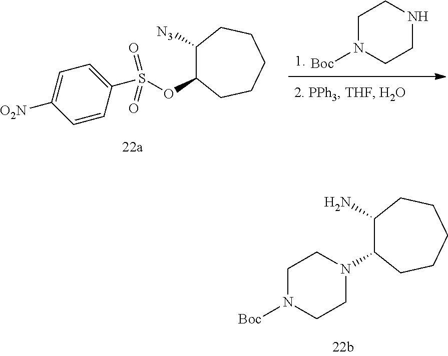 [SMILES: [N-]=[N+]=N[C@@H]1CCCCC[C@H]1OS(=O)(=O)c1ccc([N+](=O)[O-])cc1 ; CC(C)(C)OC(=O)N1CCNCC1 ; CC(C)(C)OC(=O)N1CCN([C@H]2CCCCC[C@H]2N)CC1]

The mixture of trans-2-azidocycloheptyl 4-nitrobenzenesulfonate (340 mg, 1 mmol) and tert-butyl piperazine-1-carboxylate (930 mg, 5 mmol) was heated to 150° C. for 2 h. After being cooled down to room temperature, the reaction mixture was purified by flash chromatography (silica gel, 0-50% ethyl acetate in petroleum ether) to afford cis-tert-butyl 4-(2-azidocycloheptyl)piperazine-1-carboxylate (100 mg, 31%). LC/MS ESI m/z: 324 (M+H)⁺. To a solution of cis-tert-butyl 4-(2-azidocycloheptyl)piperazine-1-carboxylate (100 mg, 0.31 mmol) in THF (8 mL) and water (2 mL) was added PPh₃ (162 mg, 0.62 mmol). The resulting mixture was heated to 50° C. overnight. The reaction was partitioned between DCM and water, organic layer was separated, aqueous layer was extracted with DCM/iPrOH (85/15) twice, the combined organic layers were dried over Na₂SO₄, filtered and concentrated. The residue was purified by flash chromatography (0˜20% MeOH in DCM) to afford cis-tert-butyl 4-(2-aminocycloheptyl)piperazine-1-carboxylate (comp. 22b, 60 mg, 65%). LC/MS ESI m/z: 298 (M+H)⁺.

### Compound 22c. cis-tert-Butyl 4-(2-((4-(N,N-dimethylsulfamoyl)phenyl)sulfonamido)cycloheptyl)piperazine-1-carboxylate

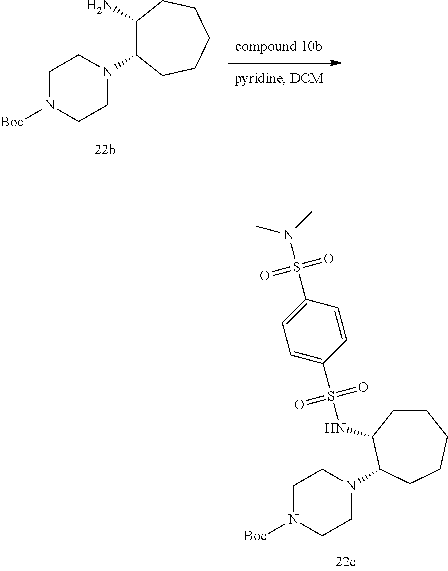 [SMILES: CC(C)(C)OC(=O)N1CCN([C@H]2CCCCC[C@H]2N)CC1 ; CN(C)S(=O)(=O)c1ccc(S(=O)(=O)N[C@@H]2CCCCC[C@@H]2N2CCN(C(=O)OC(C)(C)C)CC2)cc1]

At 0° C., to a solution of cis-tert-butyl 4-(2-aminocycloheptyl)piperazine-1-carboxylate (60 mg, 0.2 mmol) in DCM (15 mL) was added pyridine (32 mg, 0.4 mmol), followed by 4-(N,N-dimethylsulfamoyl)benzenesulfonyl chloride (comp. 10b, 113 mg, 0.4 mmol), the resulting mixture was stirred at room temperature overnight. The reaction was quenched with sat. NaHCO₃ (aq.), extracted with DCM twice, the combined organic layers were washed with brine, dried over Na₂SO₄, filtered and concentrated. The residue was purified by flash chromatography (silica gel, 0˜5% MeOH in DCM) to afford cis-tert-butyl 4-(2-((4-(N,N-dimethylsulfamoyl)phenyl)sulfonamido)cycloheptyl)piperazine-1-carboxylate (comp. 22c, 60 mg, 55%) as a white solid. LC/MS ESI m/z: 545 (M+H)⁺.

### Compound 122. Cis-N¹-(2-(4-(4-chloro-2-fluorophenyl)piperazin-1-yl)cycloheptyl)-N⁴,N⁴-dimethylbenzene-1,4-disulfonamide

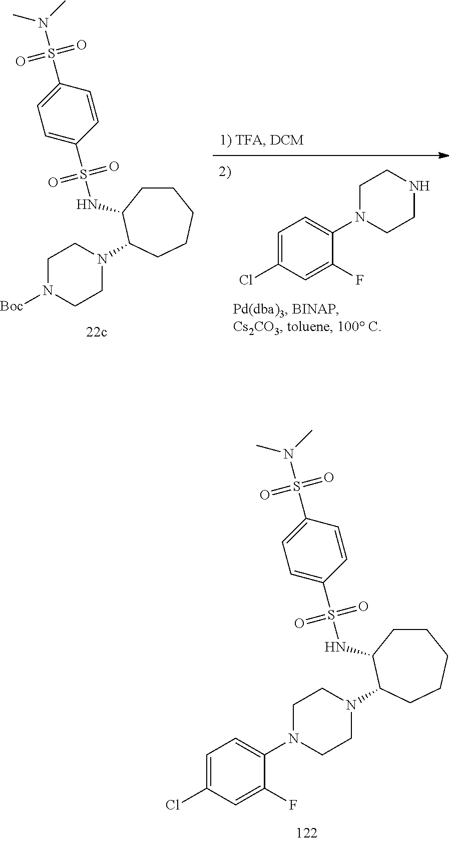 [SMILES: CN(C)S(=O)(=O)c1ccc(S(=O)(=O)N[C@@H]2CCCCC[C@@H]2N2CCN(C(=O)OC(C)(C)C)CC2)cc1 ; Fc1cc(Cl)ccc1N1CCNCC1 ; CN(C)S(=O)(=O)c1ccc(S(=O)(=O)N[C@@H]2CCCCC[C@@H]2N2CCN(c3ccc(Cl)cc3F)CC2)cc1]

At 0° C., to a solution of is-tert-butyl 4-(2-((4-(N,N-dimethylsulfamoyl)phenyl)sulfonamido)cycloheptyl)piperazine-1-carboxylate (60 mg, 0.11 mmol) in DCM (10 mL) was added TFA (1 mL), the resulting mixture was stirred at room temperature for 2 h. After removal of solvent, the residue was diluted with DCM, washed with NaHCO₃(aq.), the organic layer was extracted with DCM/iPrOH (85/15) twice, the combined organic layers were dried over Na₂SO₄, filtered and concentrated. The residue was used to the next step directly. LC/MS ESI m/z: 445 (M+H)⁺.

To a solution of cis-N1,N1-dimethyl-N4-(2-(piperazin-1-yl)cycloheptyl)benzene-1,4-disulfonamide (50 mg, 0.11 mmol) in toluene (15 mL) was added 1-(4-chloro-2-fluorophenyl)piperazine (28 mg, 0.13 mmol), Cs₂CO₃ (128 mg, 0.39 mmol), Pd₂(dba)₃ (10 mg, 0.011 mmol) and BINAP (10 mg, 0.017 mmol). The resulting mixture was heated to 100° C. overnight. After being cooled down to room temperature, solvent was removed and the residue was purified by flash chromatography (0-15% MeOH in DCM) and prep-HPLC to afford cis-N1-(2-(4-(4-chloro-2-fluorophenyl)piperazin-1-yl)cycloheptyl)-N4,N4-dimethylbenzene-1,4-disulfonamide (comp. 122, 10 mg, 16%) as a white solid. LC/MS ESI m/z: 573 (M+H)+. 1H NMR (400 MHz, CDCl 3) δ 8.06 (d, J=8.3 Hz, 2H), 7.92 (d, J=8.5 Hz, 2H), 7.08-7.03 (m, 2H), 6.86 (t, J=9.0 Hz, 1H), 3.56-3.30 (m, 1H), 3.13-3.02 (m, 4H), 2.80-2.73 (m, 8H), 2.62-2.45 (m, 3H), 1.99-1.93 (m, 1H), 1.82-1.72 (m, 6H), 1.32-1.28 (m, 1H), 1.24-1.18 (m, 1H), 1.11-1.03 (m, 1H).

### Example 23. Phenyl 3-((2-((4-(N,N-dimethylsulfamoyl)phenyl)sulfonamido)phenyl)(methyl)amino)pyrrolidine-1-carboxylate (Compound 123)

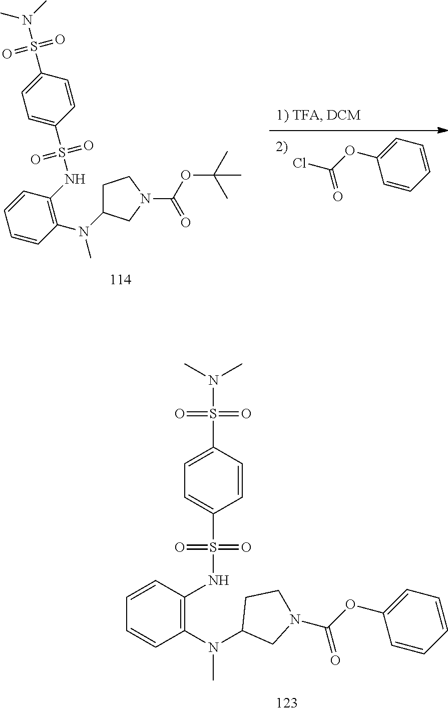 [SMILES: CN(c1ccccc1NS(=O)(=O)c1ccc(S(=O)(=O)N(C)C)cc1)C1CCN(C(=O)OC(C)(C)C)C1 ; O=C(Cl)Oc1ccccc1 ; CN(c1ccccc1NS(=O)(=O)c1ccc(S(=O)(=O)N(C)C)cc1)C1CCN(C(=O)Oc2ccccc2)C1]

At 0° C., to a solution of tert-butyl 3-((2-((4-(N,N-dimethylsulfamoyl)phenyl)sulfonamido)phenyl)(methyl)amino)pyrrolidine-1-carboxylate (comp. 114,450 mg, 0.84 mmol) in DCM (10 mL) was added TFA (2 mL), the resulting mixture was stirred at room temperature for 2 h. After removal of solvent, the residue was diluted with DCM, washed with NaHCO₃(aq.), the organic layer was extracted with DCM twice, the combined organic layers were washed with brine, dried over Na₂SO₄, filtered and concentrated. The residue was used to the next step directly. N¹,N¹-dimethyl-N⁴-(2-(methyl(pyrrolidin-3-yl)amino)phenyl)benzene-1,4-disulfonamide. LC/MS ESI m/z: 439 (M+H)⁺.

At 0° C., to a solution of N1,N1-dimethyl-N4-(2-(methyl(pyrrolidin-3-yl)amino)phenyl)benzene-1,4-disulfonamide (50 mg, 0.11 mmol) in DCM (10 mL) was added TEA (22 mg, 0.22 mmol), followed by phenyl carbonochloridate (26 mg, 0.17 mmol) in DCM (1 mL) dropwise. The resulting mixture was stirred at the same temperature for 30 min, and then was quenched with ice water, extracted with DCM twice, the combined organic layers were washed with brine, dried over Na₂SO₄, filtered and concentrated. The residue was purified by prep-HPLC to afford phenyl 3-((2-((4-(N,N-dimethylsulfamoyl)phenyl)sulfonamido)phenyl)(methyl)amino)pyrrolidine-1-carboxylate (comp. 123, 25 mg, 41%) as a white solid. LC/MS ESI m/z: 559 (M+H)+. 1H NMR (400 MHz, CDCl3) δ 8.29 (br, 1H), 8.06-8.00 (m, 2H), 7.84 (d, J=8.3 Hz, 2H), 7.58 (d, J=7.9 Hz, 1H), 7.35 (t, J=7.9 Hz, 2H), 7.22-7.07 (m, 6H), 3.74-3.38 (m, 4H), 3.32-3.13 (m, 1H), 2.70 (s, 6H), 2.36 (s, 3H), 2.00-1.91 (m, 1H), 1.75-1.68 (m, 1H).

### Example 24. Phenyl 3-((2-((4-(N,N-dimethylsulfamoyl)phenyl)sulfonamido)phenyl)(methyl)amino)pyrrolidine-1-carboxylate (Compound 124)

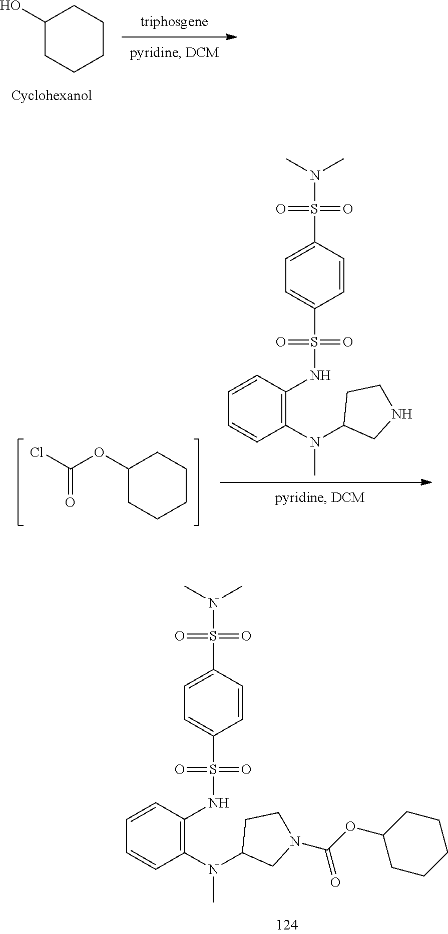

At 0° C., to a solution of triphosgene (594 mg, 2 mmol) in DCM (15 mL) was added cyclohexanol (300 mg, 3 mmol) in DCM (2 mL) dropwise, followed by pyridine (237 mg, 3 mmol). The resulting mixture was stirred at the same temperature for 30 minutes. To another solution of N1,N1-dimethyl-N4-(2-(methyl(pyrrolidin-3-yl)amino)phenyl)benzene-1,4-disulfonamide (made similarly as in Example 23, 100 mg, 0.22 mmol) in DCM (6 mL) was added pyridine (20 mg, 0.22 mmol), followed by 1/10 volume of above solution. After stirred at the same temperature for 2 h, the reaction was quenched with ice water, extracted with DCM twice, the combined organic layers were washed with brine, dried over Na₂SO₄, filtered and concentrated. The residue was purified by flash chromatography (silica gel, 0˜10% MeOH in DCM) and prep-HPLC to afford cyclohexyl 3-((2-((4-(N,N-dimethylsulfamoyl)phenyl)sulfonamido)phenyl)(methyl)amino)pyrrolidine-1-carboxylate (comp. 124, 25 mg, 20%) as a white solid. LC/MS ESI m/z: 565 (M+H)+. 1H NMR (400 MHz, CDCl3) δ 8.30 (s, 1H), 8.02 (d, J=8.2 Hz, 2H), 7.84 (d, J=8.5 Hz, 2H), 7.60-7.52 (m, 1H), 7.20-7.12 (m, 2H), 7.11-7.03 (m, 1H), 4.74-4.57 (m, 1H), 3.64-3.37 (m, 3H), 3.34-3.23 (m, 1H), 3.18-2.95 (m, 1H), 2.71 (s, 6H), 2.32 (s, 3H), 1.92-1.79 (m, 3H), 1.74-1.67 (m, 2H), 1.61-1.48 (m, 3H), 1.47-1.33 (m, 4H).

### Example 25. Effect of TRPML1 Activators on Glycophosphingolipid Levels Using Genetic Models of Disease In Vitro

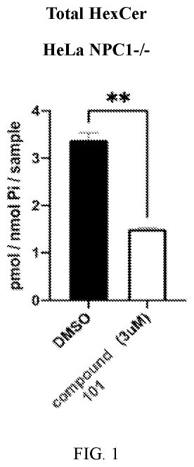

HeLa cells with the NPC1 gene knocked out via CRISPR-generated frameshifting indels were grown in standard cell culture conditions (DMEM high glucose with 10% FBS and penicillin/streptomycin). Cells were treated with the indicated concentrations of TRPML1 activators for 48 hours; media and compound were refreshed every 24 hours (FIG. 1). Two replicates per treatment were used. Cells were pelleted in PBS, frozen, and shipped to the Medical University of South Carolina (MUSC) for lipid quantitation by mass spectrometry. Lipid quantities were normalized to total inorganic phosphate in the cell sample as assessed by mass spectrometry. As shown in FIG. 1, compared to vehicle (DMSO), TRPML1 activators significantly reduced total HexosylCeramides (HexCer) (DMSO at 3.39+/−0.20 pmol HexCer/nmol Pi/sample, vs. compound 101 at 1.50+/−0.03 ng pmol HexCer/nmol Pi/sample with p=0.006). Statistical significance versus DMSO (p values indicated above) unpaired t-test.

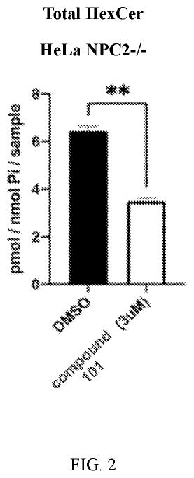

Shown in FIG. 2 are results from the same experiment described in FIG. 1, but using HeLa cells with the NPC2 gene knocked out via CRISPR. Again, TRPML1 activators significantly reduced total HexosylCeramides (DMSO at 6.45+/−0.28 HexCer/nmol Pi/sample, vs. compound 101 at 3.48+/−0.20 HexCer/nmol Pi/sample with p=0.0068).

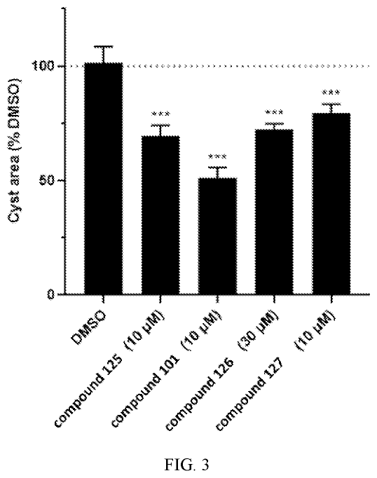

Shown in FIG. 3 are results from an experiment where mIMCD-3 cells with the PKD1 gene knocked out via CRISPR-generated frameshifting indels were seeded into 384 well plates containing Matrigel and cultured in standard growth media for 4 days until they formed spherical organoid cysts as described in Reference 1 (Booij et al., SLAS Discov. 2017 September; 22(8):974-984). TRPML1 activators and vehicle were added to the cells at the indicated concentrations, in combination with 5 uM forskolin to induce cyst swelling. Four replicates per treatment were used. Approximately half of the media in each well was replaced with fresh media containing compounds and forskolin every 24 hours. After 72 hours of compound treatment, the cysts were fixed and stained for nuclei (DAPI) and actin (fluorescent-conjugated phalloidin). The ability of the TRPML1 activators to inhibit forskolin-induced cyst swelling was assessed using the 2-dimensional cross-sectional area of each cyst as measured using a high content microscope with a 4× objective, and analyzed using proprietary Ominer software. Cyst areas were averaged within each well and normalized to the vehicle control (DMSO). TRPML1 activators from all 4 chemical classes reduced organoid swelling (DMSO cyst area 100%+/−7.1, comp. 125 cyst area 69.6%+/−4.4 and p<0.0001, comp. 101 cyst area 51.2%+/−1.6 and p<0.0001, comp. 126 cyst area 72.3%+/−2.5 and p<0.0001, comp. 127 cyst area 79.6%+/−3.8 and p<0.0001. Statistical significance versus DMSO (p values indicated above) was assessed using one-way ANOVA with Dunnett's multiple comparison test.

### Example 26. TRPML Fluorescent Assays

### TRPML1 Assay

### Cell Culture

HEK-293 Trex cells were stably transfected with a construct consisting of the human coding sequence for TRPML1 cloned into the tert-inducible plasmid pCDNA5 T/O. Mutations were introduced into the TRPML1 sequence to facilitate expression on the cell surface (Silvia Vergarajauregui, Rosa Puertollano Traffic. 2006 March; 7(3): 337-353). Briefly, the cells are cultured in 150 mm round tissue culture dishes containing 20 mL of media. The day before the assay the cells are rinsed with DPBS—Ca—Mg and then treated briefly with Trypsin-EDTA. The Trypsin-EDTA is diluted with growth media, and cells are counted. 38×10 6 cells are re-plated into 150 mm round tissue culture dishes in media containing 0.5 ug/mL doxycycline to induce expression of hTRPML1.

### Dye Loading

The day of the experiment cells are lifted from the plates as above and collected by centrifugation. The cells are then suspended in dye loading buffer consisting of Ringer's solution supplemented with 0.1% Pluronic Acid and 1 micromolar Fluo4-AM dye. Cells are loaded for ˜60 minutes in the dark with occasional mixing. The cells are collected by centrifugation, the loading media aspirated, and the cells resuspended in 25 mL Ringer's solution and incubated ˜60 minutes in the dark. The cells are again collected by centrifugation, rinsed in Ringer's Solution and resuspended to 0.2×10 6 cells/mL in modified Ringer's solution containing 10 mM calcium.

### Compound Assay Plates

Compounds are dissolved to a concentration of 10 millimolar with DMSO. Compound plates are created by dispensing compounds into 384 well black wall clear bottom plates (Greiner 781091). Positive and negative controls are included on each plate. Typically, different amounts of each compound are tested ranging from 100 nanoMoles (20 micromolar final concentration) decreasing in half-log steps to 31 picoMoles (6 nanomolar final concentration). Each concentration is typically tested in triplicate.

### Assay

50 microliters of dye-loaded cells are dispensed into each well of the compound assay plate created above. The fluorescence in each well is then determined with an excitation wavelength of 480 nM and an emission wavelength of 540 nM using either a Molecular Devices SpectraMax multimode plate reader or a Hamamatsu FDSS/uCell plate imager.

### Analysis and Statistics

The resulting fluorescence for each well is exported as an ascii file and loaded into our LIMS for analysis. The percent activity of each compound at each concentration is determined by comparison to the positive and negative control wells included in each plate.

### TRPML2 and TRPML3 Assays

Assays for TRPML2 and TRPML3 were performed as above for TRPML1, by substituting the appropriate TRPML2 or TRPML3 subtype for the TRPML1.

EC50 values were calculated using a non-linear regression of Prism. The EC50 determined for each compound using the assay is summarized in Tables 1 and 2 below.

In Tables 1 and 2 below, the activation EC50 of selected compounds is given according to the following key: A is EC50<0.3 μM; B is 0.3 μM<EC50<1 μM; C is 1 μM<EC50<5 μM; D is EC50>5 μM

TABLE 1

| | | Secondary | Secondary | Secondary |
|---|---|---|---|---|
| | | Screening: | Screening: | Screening: |
| | | Fluo-4: | Fluo-4: | Fluo-4: |
| | | hTRPML1: | hTRPML2: | hTRPML3: |
| Compound | | EC5 | EC5 | EC5 |
| Number | Structure | 0 (M) | 0 (M) | 0 (M) |
| 101 | 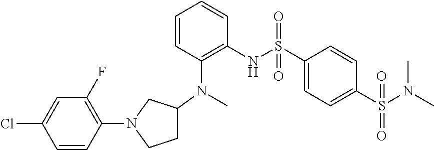 [SMILES: CN(c1ccccc1NS(=O)(=O)c1ccc(S(=O)(=O)N(C)C)cc1)C1CCN(c2ccc(Cl)cc2F)C1] | B | A | A |
| 102 | 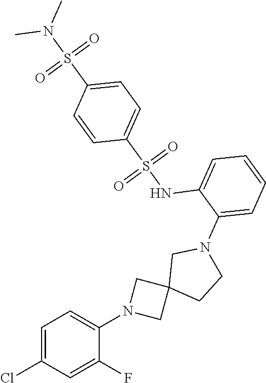 [SMILES: CN(C)S(=O)(=O)c1ccc(S(=O)(=O)Nc2ccccc2N2CCC3(CN(c4ccc(Cl)cc4F)C3)C2)cc1] | B | | |
| 103 | 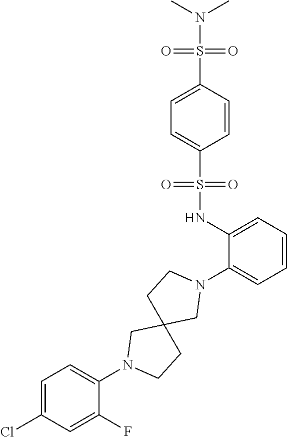 [SMILES: CN(C)S(=O)(=O)c1ccc(S(=O)(=O)Nc2ccccc2N2CCC3(CCN(c4ccc(Cl)cc4F)C3)C2)cc1] | B | | |
| 104 | 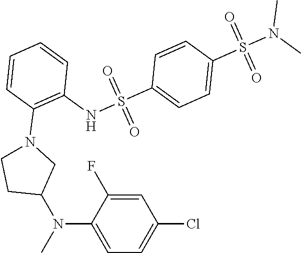 [SMILES: CN(c1ccc(Cl)cc1F)C1CCN(c2ccccc2NS(=O)(=O)c2ccc(S(=O)(=O)N(C)C)cc2)C1] | A | | |
| 105 | 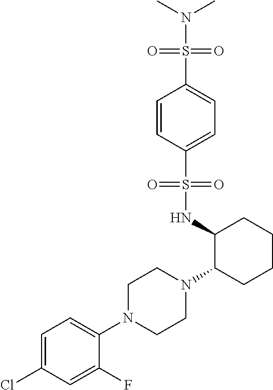 [SMILES: CN(C)S(=O)(=O)c1ccc(S(=O)(=O)N[C@H]2CCCC[C@@H]2N2CCN(c3ccc(Cl)cc3F)CC2)cc1] | B | | |
| 106 | 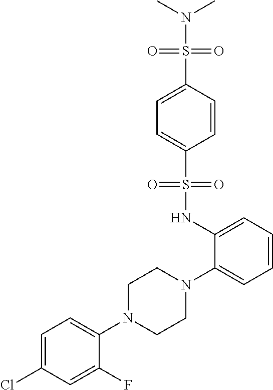 [SMILES: CN(C)S(=O)(=O)c1ccc(S(=O)(=O)Nc2ccccc2N2CCN(c3ccc(Cl)cc3F)CC2)cc1] | A | | |
| 107 | 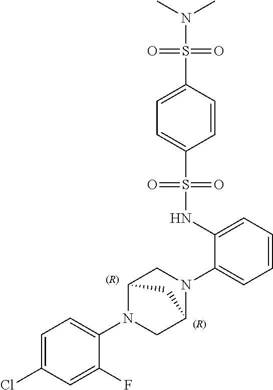 | B | | |
| 108 | 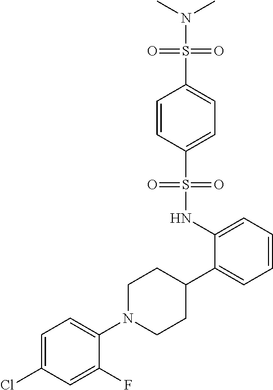 [SMILES: CN(C)S(=O)(=O)c1ccc(S(=O)(=O)Nc2ccccc2C2CCN(c3ccc(Cl)cc3F)CC2)cc1] | C | | B |
| 109 | 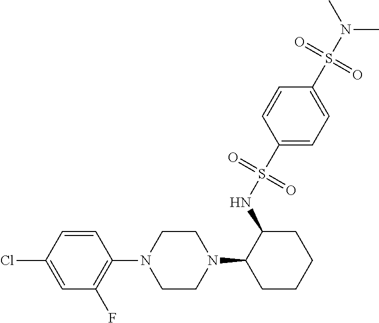 | C | | B |
| 110 | 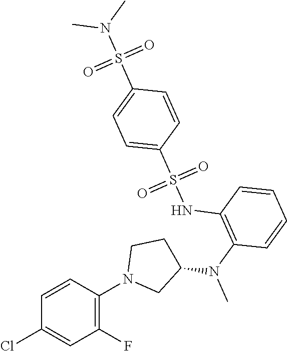 [SMILES: CN(c1ccccc1NS(=O)(=O)c1ccc(S(=O)(=O)N(C)C)cc1)[C@H]1CCN(c2ccc(Cl)cc2F)C1] | A | | A |
| 111 | 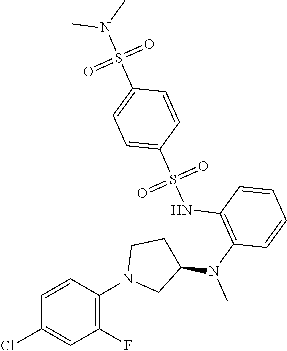 [SMILES: CN(c1ccccc1NS(=O)(=O)c1ccc(S(=O)(=O)N(C)C)cc1)[C@@H]1CCN(c2ccc(Cl)cc2F)C1] | B | | A |
| 112 | 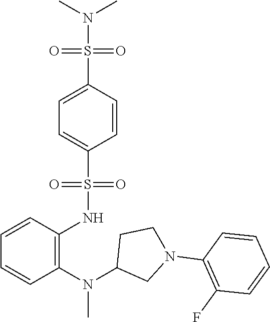 [SMILES: CN(c1ccccc1NS(=O)(=O)c1ccc(S(=O)(=O)N(C)C)cc1)C1CCN(c2ccccc2F)C1] | B | | A |
| 113 | 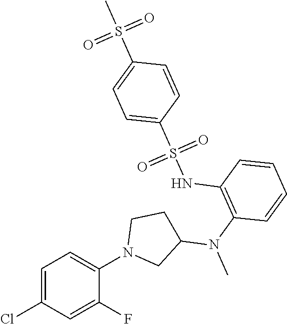 [SMILES: CN(c1ccccc1NS(=O)(=O)c1ccc(S(C)(=O)=O)cc1)C1CCN(c2ccc(Cl)cc2F)C1] | B | D | C |
| 114 | 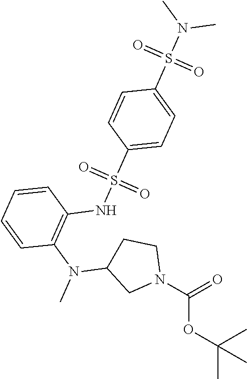 [SMILES: CN(c1ccccc1NS(=O)(=O)c1ccc(S(=O)(=O)N(C)C)cc1)C1CCN(C(=O)OC(C)(C)C)C1] | C | | B |
| 115 | 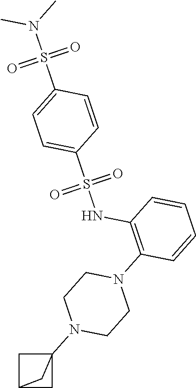 [SMILES: CN(C)S(=O)(=O)c1ccc(S(=O)(=O)Nc2ccccc2N2CCN(C34CC(C3)C4)CC2)cc1] | C | | B |
| 116 | 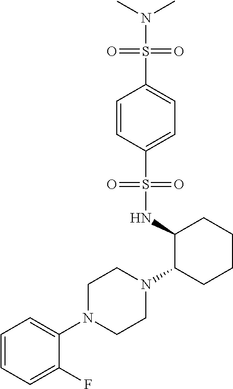 [SMILES: CN(C)S(=O)(=O)c1ccc(S(=O)(=O)N[C@H]2CCCC[C@@H]2N2CCN(c3ccccc3F)CC2)cc1] | C | | B |
| 117 | 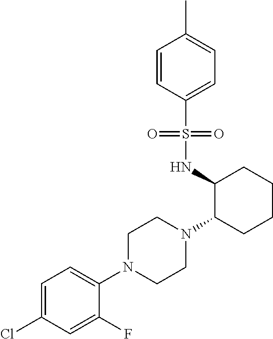 [SMILES: Cc1ccc(S(=O)(=O)N[C@H]2CCCC[C@@H]2N2CCN(c3ccc(Cl)cc3F)CC2)cc1] | B | | A |
| 118 | 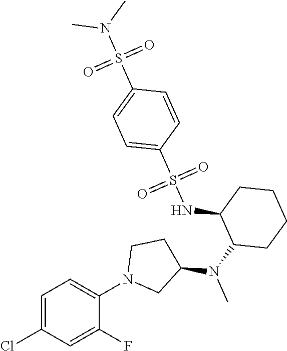 | A | D | |
| 119 | 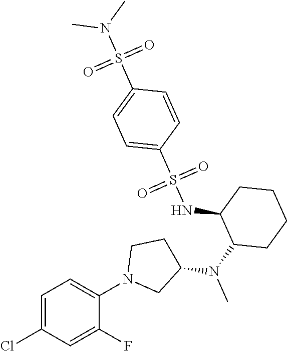 | A | D | |
| 120 | 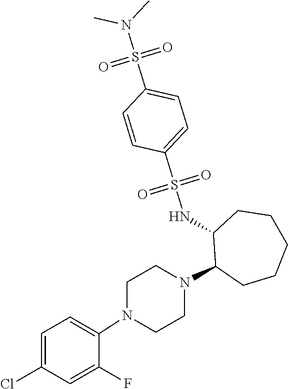 [SMILES: CN(C)S(=O)(=O)c1ccc(S(=O)(=O)N[C@@H]2CCCCC[C@H]2N2CCN(c3ccc(Cl)cc3F)CC2)cc1] | A | C | B |
| 121 | 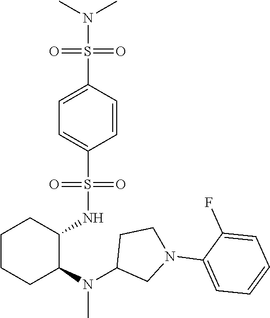 [SMILES: CN(C1CCN(c2ccccc2F)C1)[C@H]1CCCC[C@@H]1NS(=O)(=O)c1ccc(S(=O)(=O)N(C)C)cc1] | C | C | B |
| 122 | 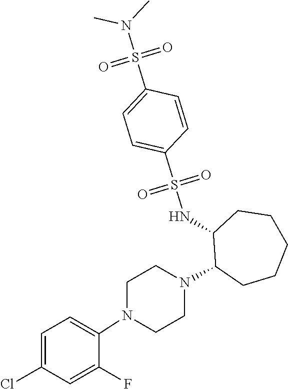 [SMILES: CN(C)S(=O)(=O)c1ccc(S(=O)(=O)N[C@@H]2CCCCC[C@@H]2N2CCN(c3ccc(Cl)cc3F)CC2)cc1] | A | C | B |
| 123 | 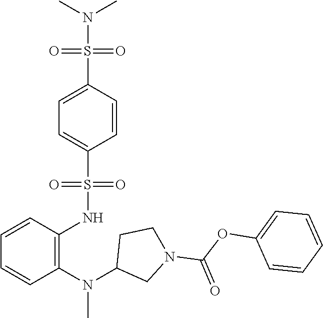 [SMILES: CN(c1ccccc1NS(=O)(=O)c1ccc(S(=O)(=O)N(C)C)cc1)C1CCN(C(=O)Oc2ccccc2)C1] | C | C | B |
| 124 | 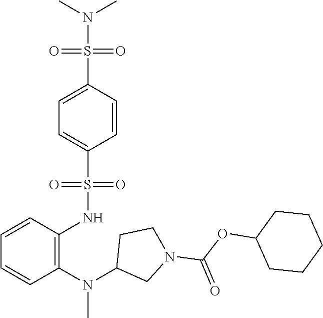 [SMILES: CN(c1ccccc1NS(=O)(=O)c1ccc(S(=O)(=O)N(C)C)cc1)C1CCN(C(=O)OC2CCCCC2)C1] | B | C | B |

In Table 2 below, the activation EC₅₀ of the compounds is given according to the following key: A is EC₅₀<10 μM; B is 10 μM<EC₅₀≤20 μM; C is 20 μM<EC₅₀≤30 μM; D is EC₅₀>30 μM.

TABLE 2

| Compound | Structure | TRPML1 | TRPML2 | TRPML3 |
|---|---|---|---|---|
| 125 | 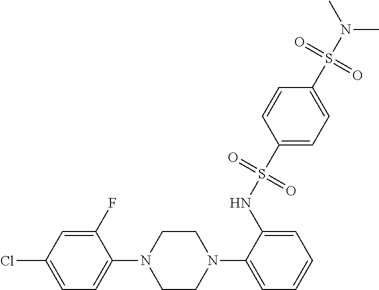 [SMILES: CN(C)S(=O)(=O)c1ccc(S(=O)(=O)Nc2ccccc2N2CCN(c3ccc(Cl)cc3F)CC2)cc1] | B (453 nm- GCaMP surface assay) | C | D |
| 126 (MLSA1) | 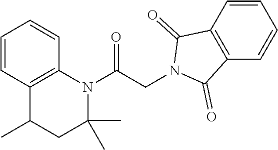 [SMILES: CC1CC(C)(C)N(C(=O)CN2C(=O)c3ccccc3C2=O)c2ccccc21] | B/C/D 3.52 uM | B/C/D 5.41 uM | A 1.58 uM |
| 127 (MK-6-83) | 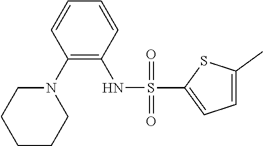 [SMILES: Cc1ccc(S(=O)(=O)Nc2ccccc2N2CCCCC2)s1] | — (2.64 um- GCaMP surface assay) | — | — |

### EQUIVALENTS

The disclosures of each and every patent, patent application, and publication cited herein are hereby incorporated herein by reference in their entirety. While this disclosure has been disclosed with reference to specific aspects, it is apparent that other aspects and variations of this disclosure may be devised by others skilled in the art without departing from the true spirit and scope of the disclosure. The appended claims are intended to be construed to include all such aspects and equivalent variations.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this disclosure has been particularly shown and described with references to preferred embodiments thereof, it will be understood by those skilled in the art that various changes in form and details may be made therein without departing from the scope of the disclosure encompassed by the appended claims.

1. A method of treating a ciliopathy, the method comprising administering to a patient in need thereof a therapeutically effective amount of a compound or pharmaceutical composition thereof, wherein the compound modulates a TRPML ion channel.

2. The method of claim 1, wherein the compound activates the TRPML channel.

3. The method of claim 1 or 2, wherein the TRPML channel is TRPML1.

4. The method of claim 3, wherein the compound achieves a maximal current obtained with 30 μM ML-SA1 in a patch clamp assay for TRPML1 which is at least 10 fold the maximal current achieved for any other TRPML channel.

5. The method of any one of claims 1-4, wherein the ciliopathy is selected from the group consisting of polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy, Ellis van Creveld syndrome, Sensenbrenner syndrome, and primary ciliary dyskinesia.

6. The method of claim 5, wherein the ciliopathy is polycystic kidney disease or pancreatic cysts associated with autosomal dominant polycystic kidney disease

7. The method of claim 6 wherein the ciliopathy is autosomal dominant polycystic kidney disease, autosomal recessive polycystic kidney disease, or pancreatic cysts associated with autosomal dominant polycystic kidney disease.

8. The method of claim 7, wherein the ciliopathy is autosomal dominant polycystic kidney disease.

9. The method of any one of claims 1-8, the method further comprising use of a second therapeutic agent.

10. The method of claim 9, wherein the second therapeutic agent is selected from the group consisting of an mTOR inhibitor, V2 receptor antagonist, tyrosine kinase inhibitor, somatostatin analog, glucosylceramide synthase inhibitor, microRNA-17 inhibitor, siRNA against p53, KEAP1-Nrf2 activator, xanthine oxidase inhibitor, PPARγ agonist, metformin, and beta hydroxybutyrate.

11. The method of claim 9, wherein the second therapeutic agent is selected from the group consisting of tolvaptan, lixivaptan, mozavaptan, satavaptan, sirolimus, tacrolimus, everolimus, bosutinib, tesavatinib, imatinib, gefitinib, erlotinib, dasatinib, octreotide, pasireotide, venglustat, eliglustat, miglustat, microRNA-17 inhibitor, bardoxolone methyl, allopurinol, oxypurinol, pioglitazone, rosiglitazone, lobeglitazone, metformin, and beta hydroxybutyrate.

12. The method of claim 9, wherein the second therapeutic agent is selected from the group consisting of an immunomodulator, a calcineurin inhibitor, a renin angiotensin aldosterone system inhibitor, an antiproliferative agent, an alkylating agent, a corticosteroid, an angiotensin converting enzyme inhibitor, an adrenocorticotropic hormone stimulant, an angiotensin receptor blocker, a sodium-glucose transport protein 2 inhibitor, a dual sodium-glucose transport protein 1/2 inhibitor, a nuclear Factor-1 (erythroid-derived 2)-like 2 agonist, a chemokine receptor 2 inhibitor, a chemokine receptor 5 inhibitor, an endothelin 1 receptor antagonist, a beta blocker, a mineralocorticoid receptor antagonist, a loop or thiazide diuretic, a calcium channel blocker, a statin, a short-intermediate or long-acting insulin, a dipeptidyl peptidase 4 inhibitor, a glucagon-like peptide 1 receptor agonist, a sulfonylurea, an apoptosis signal-regulating kinase-1, a chymase inhibitor, a selective gly cation inhibitor, a renin inhibitor, an interleukin-33 inhibitor, a farnesoid X receptor agonist, a soluble guanylate cyclase stimulator, a thromboxane receptor antagonist, a xanthine oxidase inhibitor, an erythropoietin receptor agonist, a cannabinoid receptor type 1 inverse agonist, a NADPH oxidase inhibitor, an anti-vascular endothelial growth factor B, an anti-fibrotic agent, a neprilysin inhibitor, a dual CD80/CD86 inhibitor, a CD40 antagonist, a cellular cholesterol and lipid blocker, a PDGFR antagonist, a Slit guidance ligand 2, an APOL1 inhibitor, an Nrl2 activator/NF-kB inhibitor, a somatostatin receptor agonist, a PPAR gamma agonist, a AMP activated protein kinase stimulator, a tyrosine kinase inhibitor, a glucosylceramide synthase inhibitor, an arginine vasopressin receptor 2 antagonist, a xanthine oxidase inhibitor, a vasopressin receptor 2 antagonist, anti-amyloid beta antibodies, anti-Tau antibodies, anti-synuclein antibodies, dopamine precursors (e.g. L-DOPA), dopamine agonists (e.g. bromocriptine, cabergoline, pergolide, pramipexole and apomorphine), MAO-B inhibitors (e.g. rasagiline and selegiline), anticholinergics (e.g. orphenadrine, procyclidine and trihexyphenidyl), enhancers of b-glucocerebrosidase activity (e.g. ambroxol and afegostat), amantadine, and agents capable of treating Alzheimer's (e.g., acetylcholinesterase inhibitors such as tacrine, rivastigmine, galantamine, donepezil, and memantine).

13. The method of claim 9, wherein the second therapeutic agent is selected from the group consisting of COX inhibitors including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam; treatments for pulmonary hypertension including prostanoids (epoprostenol, iloprost, and treprostinil), endothelin receptor antagonists (bosentan, ambrisentan, and macitentan), phosphodiesterase-5 inhibitors (sildenafil and tadalafil), and sGC stimulators (riociguat); rho-kinase inhibitors, such as Y-27632, fasudil, and H-1152P; epoprostenol derivatives, such as prostacyclin, treprostinil, beraprost, and iloprost; serotonin blockers, such as sarpogrelate; endothelin receptor antagonists, such as besentan, sitaxsentan, ambrisentan, and TBC3711; PDE inhibitors, such as sildenafil, tadalafil, udenafil, and vardenafil, calcium channel blockers, such as amlodipine, bepridil, clentiazem, diltiazem, fendiline, gallopamil, mibefradil, prenylamine, semotiadil, terodiline, verapamil, aranidipine, bamidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, and perhexiline; tyrosine kinase inhibitors, such as imatinib; inhaled nitric oxide and nitric oxide-donating agents, such as inhaled nitrite; IκB inhibitors, such as IMD 1041; prostacyclin receptor agonists, such as selexipag; stimulators of hematopoiesis, such as TXA 127 (angiotensin (1-7)), darbepoetin alfa, erythropoetin, and epoetin alfa; anticoagulants and platelet-inhibiting agents, and diuretics; dietary and nutritional supplements such as acetyl-L-carnitine, octacosanol, evening primrose oil, vitamin B6, tyrosine, phenylalanine, vitamin C, L-dopa; immunosuppressants (for transplants and autoimmune-related RKD); anti-hypertensive drugs (for high blood pressure-related RKD, e.g., angiotensin-converting enzyme inhibitors and angiotensin receptor blockers), insulin (for diabetic RKD); lipid/cholesterol-lowering agents (e.g., HMG-CoA reductase inhibitors such as atorvastatin or simvastatin); and treatments for hyperphosphatemia or hyperparathyroidism associated with CKD (e.g., sevelamer acetate, cinacalcet).

14. The method of any one of claims 1-13, wherein the compound is a compound disclosed in the specification, optionally in table 1 or table 2.
